SEQUENCE LISTING (71 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, unidentified
  source 1..226: note = Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, unidentified
  source 1..608: note = Phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, unidentified
  source 1..970: note = Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, unidentified
  source 1..439: note = Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - DNA, 3595 bp, synthetic construct
  source 1..3595: mol_type = other DNA
gccatcagattgtgtttgttagtcgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagcatgtagga

SEQ ID NO: 10 - DNA, 10 bp, synthetic construct
  source 1..10: mol_type = other DNA
  misc_feature 1: note = thymine joined to 5Biotin triethyleneglycol (TEG)
  misc_feature 10: note = thymine joined to iSp18 spacer which is joined to another nucleic acid
tttttttttt

SEQ ID NO: 11 - DNA, 65 bp, synthetic construct
  source 1..65: mol_type = other DNA
  misc_feature 1: note = 5Phosphate
  modified_base 23: mod_base = OTHER; note = bridged nucleic acid
  modified_base 24: mod_base = OTHER; note = bridged nucleic acid methyl-C
  modified_base 25..27: mod_base = OTHER; note = bridged nucleic acid
gcaatacgtaactgaacgaagtacatttttgaggcgagcggtcaatttttttttttttttttttt

SEQ ID NO: 12 - DNA, 60 bp, synthetic construct
  source 1..60: mol_type = other DNA
  misc_feature 1: note = 5Phosphate
tgcaatacgtaactgaacgaagtacattaatgtacttcgttcagttacgtattgcatcct

SEQ ID NO: 13 - DNA, 91 bp, synthetic construct
  source 1..91: mol_type = other DNA
  misc_feature 1: note = 5Phosphate
tgcaatacgtaactgaacgaagtacatttttttgaagatagagcgattttttttttttttttgtacttcgttcagttacgtattgcatcct

SEQ ID NO: 14 - DNA, 88 bp, synthetic construct
  source 1..88: mol_type = other DNA
  misc_feature 1: note = 5Phosphate
tgcaatacgtaactgaacgaagtacatttttttgaagatagagcgattttttttttttttttgtacttcgttcagttacgtattgcat

SEQ ID NO: 15 - DNA, 92 bp, synthetic construct
  source 1..92: mol_type = other DNA
  misc_feature 1: note = 5Phosphate
  modified_base 52..54: mod_base = OTHER; note = thymine modified by fluorophore
tgcaatacgtaactgaacgaagtacatttttttgaagatagagcgatttttttttttttttttgtacttcgttcagttacgtattgcatcct

SEQ ID NO: 16 - DNA, 13 bp, synthetic construct
  source 1..13: mol_type = other DNA
  modified_base 1: mod_base = OTHER; note = bridged nucleic acid
  modified_base 2: mod_base = OTHER; note = bridged nucleic acid methyl-C
  modified_base 4: mod_base = OTHER; note = bridged nucleic acid methyl-C
  modified_base 5: mod_base = OTHER; note = bridged nucleic acid
  modified_base 3: mod_base = OTHER; note = bridged nucleic acid
tcgctctatcttc

SEQ ID NO: 17 - DNA, 35 bp, synthetic construct
  source 1..35: mol_type = other DNA
  misc_feature 35: note = thymine joined to iSp18 spacer which is joined to another nucleic acid
gttattcaagacttctttaatacactttttttttt

SEQ ID NO: 18 - DNA, 61 bp, synthetic construct
  source 1..61: mol_type = other DNA
  misc_feature 1: note = 5Phosphate
ggttaaacacccaagcagacgcctttgaggcgagcggtcaatttttttttttttttttttt

SEQ ID NO: 19 - DNA, 27 bp, synthetic construct
  source 1..27: mol_type = other DNA
  modified_base 23: mod_base = OTHER; note = bridged nucleic acid
  modified_base 27: mod_base = OTHER; note = bridged nucleic acid
  modified_base 24: mod_base = OTHER; note = bridged nucleic acid methyl-C
  modified_base 25..26: mod_base = OTHER; note = bridged nucleic acid
gcaatacgtaactgaacgaagtacatt

SEQ ID NO: 20 - DNA, 25 bp, synthetic construct
  source 1..25: mol_type = other DNA
  modified_base 1: mod_base = OTHER; note = bridged nucleic acid
  modified_base 2..5: mod_base = OTHER; note = bridged nucleic acid
  modified_base 23..24: mod_base = OTHER; note = bridged nucleic acid
  modified_base 25: mod_base = OTHER; note = bridged nucleic acid methyl-C
gtgtattaaagaagtcttgaataac

SEQ ID NO: 21 - DNA, 25 bp, synthetic construct
  source 1..25: mol_type = other DNA
gtgtattaaagaagtcttgaataac

SEQ ID NO: 22 - DNA, 41 bp, synthetic construct
  source 1..41: mol_type = other DNA
  misc_feature 1: note = 5Phosphate
  misc_feature 41: note = adenine joined to 30x C3 spacer
ggttaaacacccaagcagacgcctttgaggcgagcggtcaa

SEQ ID NO: 23 - protein, 65 aa, synthetic construct
GTGSGAWKEWLERKVGEGRARRLIEYFGSAGEVGKLVENAEVSKLLEVPGIGDEAVARLVPGGSS

SEQ ID NO: 24 - protein, 299 aa, unidentified
  source 1..299: note = Bacteriophage RB69
MFKRKSTADLAAQMAKLNGNKGFSSEDKGEWKLKLDASGNGQAVIRFLPAKTDDALPFAILVNHGFKKNGKWYIETCSSTHGDYDSCPVCQYISKNDLYNTNKTEYSQLKRKTSYWANILVVKDPQAPDNEGKVFKYRFGKKIWDKINAMIAVDTEMGETPVDVTCPWEGANFVLKVKQVSGFSNYDESKFLNQSAIPNIDDESFQKELFEQMVDLSEMTSKDKFKSFEELNTKFNQVLGTAALGGAAAAAASVADKVASDLDDFDKDMEAFSSAKTEDDFMSSSSSDDGDLDDLLAGL

SEQ ID NO: 25 - protein, 232 aa, unidentified
  source 1..232: note = Bacteriophage T7
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 26 - protein, 324 aa, unidentified
  source 1..324: note = Herpes virus 1
MDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQKHHHHHH

SEQ ID NO: 27 - protein, 251 aa, unidentified
  source 1..251: note = Crenarchaeota sp.
MFKIVYPNAKDFFSFINSITNVTDSIILNFTEDGIFSRHLTEDKVLMAIMRIPKDVLSEYSIDSPTSVKLDVSSVKKILSKASSKKATIELTETDSGLKIIIRDEKSGAKSTIYIKAEKGQVEQLTEPKVNLAVNFTTDESVLNVIAADVTLVGEEMRISTEEDKIKIEAGEEGKRYVAFLMKDKPLKELSIDTSASSSYSAEMFKDAVKGLRGFSAPTMVSFGENLPMKIDVEAVSGGHMIFWIAPRLLE

SEQ ID NO: 28 - protein, 245 aa, unidentified
  source 1..245: note = Crenarchaeota sp.
MKAKVIDAVSFSYILRTVGDFLSEANFIVTKEGIRVSGIDPSRVVFLDIFLPSSYFEGFEVSQEKEIIGFKLEDVNDILKRVLKDDTLILSSNESKLTLTFDGEFTRSFELPLIQVESTQPPSVNLEFPFKAQLLTITFADIIDELSDLGEVLNIHSKENKLYFEVIGDLSTAKVELSTDNGTLLEASGADVSSSYGMEYVANTTKMRRASDSMELYFGSQIPLKLRFKLPQEGYGDFYIAPRAD

SEQ ID NO: 29 - protein, 246 aa, unidentified
  source 1..246: note = Crenarchaeota sp.
MKVVYDDVRVLKDIIQALARLVDEAVLKFKQDSVELVALDRAHISLISVNLPREMFKEYDVNDEFKFGFNTQYLMKILKVAKRKEAIEIASESPDSVIINIIGSTNREFNVRNLEVSEQEIPEINLQFDISATISSDGFKSAISEVSTVTDNVVVEGHEDRILIKAEGESEVEVEFSKDTGGLQDLEFSKESKNSYSAEYLDDVLSLTKLSDYVKISFGNQKPLQLFFNMEGGGKVTYLLAPKVLE

SEQ ID NO: 30 - protein, 318 aa, unidentified
  source 1..318: note = Herpes virus 1
TDSPGGVAPASPVEDASDASLGQPEEGAPCQVVLQGAELNGILQAFAPLRTSLLDSLLVMGDRGILIHNTIFGEQVFLPLEHSQFSRYRWRGPTAAFLSLVDQKRSLLSVFRANQYPDLRRVELAITGQAPFRTLVQRIWTTTSDGEAVELASETLMKRELTSFVVLVPQGTPDVQLRLTRPQLTKVLNATGADSATPTTFELGVNGKFSVFTTSTCVTFAAREEGVSSSTSTQVQILSNALTKAGQAAANAKTVYGENTHRTFSVVVDDCSMRAVLRRLQVGGGTLKFFLTTPVPSLCVTATGPNAVSAVFLLKPQK

SEQ ID NO: 31 - protein, 55 aa, synthetic construct
WKEWLERKVGEGRARRLIEYFGSAGEVGKLVENAEVSKLLEVPGIGDEAVARLVP

SEQ ID NO: 32 - protein, 107 aa, synthetic construct
WKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILG

SEQ ID NO: 33 - protein, 132 aa, Homo sapiens
ESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 34 - protein, 123 aa, unidentified
  source 1..123: note = Phi29
ENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 35 - protein, 177 aa, Escherichia coli
ASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGRQGGGAPAGGNIGGGQPQGGWGQPQQPQGGNQFSGGAQSRPQQSAPAAPSNEPPMDFDDDIPF

SEQ ID NO: 36 - protein, 301 aa, unidentified
  source 1..301: note = Bacteriophage T4
MFKRKSTAELAAQMAKLNGNKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKADKVADDLDAFNVDDFNTKTEDDFMSSSSGSSSSADDTDLDDLLNDL

SEQ ID NO: 37 - protein, 984 aa, Methanopyrus kandleri
MALVYDAEFVGSEREFEEERETFLKGVKAYDGVLATRYLMERSSSAKNDEELLELHQNFILLTGSYACSIDPTEDRYQNVIVRGVNFDERVQRLSTGGSPARYAIVYRRGWRAIAKALDIDEEDVPAIEVRAVKRNPLQPALYRILVRYGRVDLMPVTVDEVPPEMAGEFERLIERYDVPIDEKEERILEILRENPWTPHDEIARRLGLSVSEVEGEKDPESSGIYSLWSRVVVNIEYDERTAKRHVKRRDRLLEELYEHLEELSERYLRHPLTRRWIVEHKRDIMRRYLEQRIVECALKLQDRYGIREDVALCLARAFDGSISMIATTPYRTLKDVCPDLTLEEAKSVNRTLATLIDEHGLSPDAADELIEHFESIAGILATDLEEIERMYEEGRLSEEAYRAAVEIQLAELTKKEGVGRKTAERLLRAFGNPERVKQLAREFEIEKLASVEGVGERVLRSLVPGYASLISIRGIDRERAERLLKKYGGYSKVREAGVEELREDGLTDAQIRELKGLKTLESIVGDLEKADELKRKYGSASAVRRLPVEELRELGFSDDEIAEIKGIPKKLREAFDLETAAELYERYGSLKEIGRRLSYDDLLELGATPKAAAEIKGPEFKFLLNIEGVGPKLAERILEAVDYDLERLASLNPEELAEKVEGLGEELAERVVYAARERVESRRKSGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 38 - protein, 299 aa, Methanopyrus kandleri
SGRQERSEEEWKEWLERKVGEGRARRLIEYFGSAGEVGKLVENAEVSKLLEVPGIGDEAVARLVPGYKTLRDAGLTPAEAERVLKRYGSVSKVQEGATPDELRELGLGDAKIARILGLRSLVNKRLDVDTAYELKRRYGSVSAVRKAPVKELRELGLSDRKIARIKGIPETMLQVRGMSVEKAERLLERFDTWTKVKEAPVSELVRVPGVGLSLVKEIKAQVDPAWKALLDVKGVSPELADRLVEELGSPYRVLTAKKSDLMRVERVGPKLAERIRAAGKRYVEERRSRRERIRRKLRG

SEQ ID NO: 39 - protein, 853 aa, Escherichia coli
MSAIENFDAHTPMMQQYLRLKAQHPEILLFYRMGDFYELFYDDAKRASQLLDISLTKRGASAGEPIPMAGIPYHAVENYLAKLVNQGESVAICEQIGDPATSKGPVERKVVRIVTPGTISDEALLQERQDNLLAAIWQDSKGFGYATLDISSGRFRLSEPADRETMAAELQRTNPAELLYAEDFAEMSLIEGRRGLRRRPLWEFEIDTARQQLNLQFGTRDLVGFGVENAPRGLCAAGCLLQYAKDTQRTTLPHIRSITMEREQDSIIMDAATRRNLEITQNLAGGAENTLASVLDCTVTPMGSRMLKRWLHMPVRDTRVLLERQQTIGALQDFTAGLQPVLRQVGDLERILARLALRTARPRDLARMRHAFQQLPELRAQLETVDSAPVQALREKMGEFAELRDLLERAIIDTPPVLVRDGGVIASGYNEELDEWRALADGATDYLERLEVRERERTGLDTLKVGFNAVHGYYIQISRGQSHLAPINYMRRQTLKNAERYIIPELKEYEDKVLTSKGKALALEKQLYEELFDLLLPHLEALQQSASALAELDVLVNLAERAYTLNYTCPTFIDKPGIRITEGRHPVVEQVLNEPFIANPLNLSPQRRMLIITGPNMGGKSTYMRQTALIALMAYIGSYVPAQKVEIGPIDRIFTRVGAADDLASGRSTFMVEMTETANILHNATEYSLVLMDEIGRGTSTYDGLSLAWACAENLANKIKALTLFATHYFELTQLPEKMEGVANVHLDALEHGDTIAFMHSVQDGAASKSYGLAVAALAGVPKEVIKRARQKLRELESISPNAAATQVDGTQMSLLSVPEETSPAVEALENLDPDSLTPRQALEWIYRLKSLV

SEQ ID NO: 40 - protein, 64 aa, unidentified
  source 1..64: note = Sufolobus solfataricus
MATVKFKYKGEEKEVDISKIKKVWRVGKMISFTYDEGGGKTGRGAVSEKDAPKELLQMLEKQKK

SEQ ID NO: 41 - protein, 99 aa, unidentified
  source 1..99: note = Sulfolobus solfataricus P2
EKMSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 42 - protein, 88 aa, unidentified
  source 1..88: note = Sulfolobus solfataricus P2
TEKLNEIVVRKTKNVEDHVLDVIVLFNQGIDEVILKGTGREISKAVDVYNSLKDRLGDGVQLVNVQTGSEVRDRRRISYILLRLKRVY

SEQ ID NO: 43 - protein, 107 aa, Escherichia coli
AQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 44 - protein, 237 aa, unidentified
  source 1..237: note = Enterobacteria phage lambda
MSTKKKPLTQEQLEDARRLKAIYEKKKNELGLSQESVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 45 - protein, 60 aa, unidentified
  source 1..60: note = Crenarchaeota sp.
MSSGKKPVKVKTPAGKEAELVPEKVWALAPKGRKGVKIGLFKDPETGKYFRHKLPDDYPI

SEQ ID NO: 46 - protein, 136 aa, Homo sapiens [sequence omitted]

SEQ ID NO: 47 - protein, 89 aa, unidentified
  source 1..89: note = Enterobacteria phage T4
MAKKEMVEFDEAIHGEDLAKFIKEASDHKLKISGYNELIKDIRIRAKDELGVDGKMFNRLLALYHKDNRDVFEAETEEVVELYDTVFSK

SEQ ID NO: 48 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 49 - protein, 375 aa, unidentified
  source 1..375: note = Citromicrobium bathyomarinum JL354
MKATIERATLLRCLSHVQSVVERRNTIPILSNVLIDADAGGGVKVMATDLDLQVVETMTAASVESAGAITVSAHLLFDIARKLPDGSQVSLETADNRMVVKAGRSRFQLPTLPRDDFPVIVEGELPTSFELPARELAEMIDRTRFAISTEETRYYLNGIFLHVSDEARPVLKAAATDGHRLARYTLDRPEGAEGMPDVIVPRKAVGELRKLLEEALDSNVQIDLSASKIRFALGGEGGVVLTSKLIDGTFPDYSRVIPTGNDKLLRLDPKAFFQGVDRVATIATEKTRAVKMGLDEDKVTLSVTSPDNGTAAEEIAAEYKAEGFEIGFNANYLKDILGQIDSDTVELHLADAGAPTLIRRDENSPALYVLMPMRV

SEQ ID NO: 50 - DNA, 35 bp, synthetic construct; identical to SEQ ID NO: 17
  source 1..35: mol_type = other DNA
  misc_feature 35: note = thymine joined to iSp9 spacer which is joined to another nucleic acid

SEQ ID NO: 51 - DNA, 43 bp, synthetic construct
  source 1..43: mol_type = other DNA
gtgtattaaagaagtcttgaataactttgaggcgagcggtcaa

SEQ ID NO: 52 - DNA, 30 bp, synthetic construct
  source 1..30: mol_type = other DNA
  modified_base 26: mod_base = OTHER; note = bridged nucleic acid
  modified_base 30: mod_base = OTHER; note = bridged nucleic acid
  modified_base 27: mod_base = OTHER; note = bridged nucleic acid methyl-C
  modified_base 28..29: mod_base = OTHER; note = bridged nucleic acid
tttgcaatacgtaactgaacgaagtacatt

SEQ ID NO: 53 - DNA, 26 bp, synthetic construct
  source 1..26: mol_type = other DNA
  misc_feature 1: note = 5Phosphate
  misc_feature 26: note = thymine joined to 30x C3 spacer
ggttaaacacccaagcagacgccttt

SEQ ID NO: 54 - DNA, 32 bp, synthetic construct
  source 1..32: mol_type = other DNA
  modified_base 27..32: mod_base = um
  misc_feature 1: note = 5Phosphate
  misc_feature 32: note = thymine joined to 30x C3 spacer
ggttaaacacccaagcagacgccttttttttt

SEQ ID NO: 55 - DNA, 25 bp, synthetic construct
  source 1..25: mol_type = other DNA
  misc_feature 1: note = 5Phosphate
  modified_base 24..25: mod_base = um
  misc_feature 25: note = thymine joined to 30x C3 spacer
ggttaaacacccaagcagacgcctt

SEQ ID NO: 56 - DNA, 33 bp, synthetic construct
  source 1..33: mol_type = other DNA
  misc_feature 1: note = 5Phosphate
  misc_feature 33: note = thymine joined to 20x C3 spacer which is joined to another nucleic acid
ggcgtctgcttgggtgtttaacctttttttttt

SEQ ID NO: 57 - DNA, 27 bp, synthetic construct
  source 1..27: mol_type = other DNA
  misc_feature 1: note = thymine joined to 5Biotin triethyleneglycol (TEG)
tttggttaaacacccaagcagacgcct

SEQ ID NO: 58 - DNA, 15 bp, synthetic construct
  source 1..15: mol_type = other DNA
  misc_feature 15: note = adenine joined to 2x Spacer 18 which is joined to another nucleic acid
gaggcgagcggtcaa

SEQ ID NO: 59 - DNA, 44 bp, synthetic construct
  source 1..44: mol_type = other DNA
  misc_feature 1: note = 5Phosphate
  misc_feature 44: note = thymine joined to 20x C3 Spacer which is joined to another nucleic acid
ggactatacaaggcgtctgcttgggtgtttaacctttttttttt

SEQ ID NO: 60 - DNA, 35 bp, synthetic construct
  source 1..35: mol_type = other DNA
ggttaaacacccaagcagacgccttgtatagtcct

SEQ ID NO: 61 - DNA, 33 bp, synthetic construct; identical to SEQ ID NO: 56
  source 1..33: mol_type = other DNA
  misc_feature 1: note = 5Phosphate
  misc_feature 33: note = thymine joined to 20x C3 spacer which is joined to another nucleic acid

SEQ ID NO: 62 - DNA, 27 bp, synthetic construct
  source 1..27: mol_type = other DNA
  misc_feature 1: note = thymine joined to 5Biotin triethyleneglycol (TEG)
tttggttaaacacccaagcagacgcct

SEQ ID NO: 63 - DNA, 15 bp, synthetic construct
  source 1..15: mol_type = other DNA
  misc_feature 15: note = adenine joined to 2x Spacer 18 which is joined to another nucleic acid
gaggcgagcggtcaa

SEQ ID NO: 64 - DNA, 28 bp, synthetic construct
  source 1..28: mol_type = other DNA
  misc_feature 1: note = adenine joined to iSp18 spacer which is joined to another nucleic acid
aatgtacttcgttcagttacgtattgct

SEQ ID NO: 65 - DNA, 54 bp, synthetic construct
  source 1..54: mol_type = other DNA
  misc_feature 1: note = adenine joined to iSp18 spacer which is joined to another nucleic acid
aatgtacttcgttcagttacgtattgctttggcgtctgcttgggtgtttaacct

SEQ ID NO: 66 - DNA, 54 bp, synthetic construct; identical to SEQ ID NO: 65
  source 1..54: mol_type = other DNA
  misc_feature 1: note = adenine joined to iSp9 spacer which is joined to another nucleic acid

SEQ ID NO: 67 - DNA, 47 bp, synthetic construct
  source 1..47: mol_type = other DNA
  misc_feature 1: note = adenine joined to 20x C3 spacer which is joined to another nucleic acid
aatgtacttcgttcagttacgtattgctttttttttttttttttttt

SEQ ID NO: 68 - DNA, 27 bp, synthetic construct
  source 1..27: mol_type = other DNA
  misc_feature 1: note = guanine joined to 2x Spacer 18 which is joined to another nucleic acid
  modified_base 23..27: mod_base = OTHER; note = locked nucleotides
gcaatacgtaactgaacgaagtacatt

SEQ ID NO: 69 - DNA, 47 bp, synthetic construct; identical to SEQ ID NO: 67
  source 1..47: mol_type = other DNA
  misc_feature 1: note = adenine joined to 20x C3 Spacer which is joined to another nucleic acid

SEQ ID NO: 70 - DNA, 47 bp, synthetic construct; identical to SEQ ID NO: 67
  source 1..47: mol_type = other DNA
  misc_feature 1: note = adenine joined to 20x C3 spacer which is joined to another nucleic acid

SEQ ID NO: 71 - DNA, 27 bp, synthetic construct
  source 1..27: mol_type = other DNA
  misc_feature 1: note = guanine joined to 2x Spacer 18 which is joined to another nucleic acid
  modified_base 23..27: mod_base = OTHER; note = locked nucleotides
gcaatacgtaactgaacgaagtacatt

(19) United States
(12) Patent Application Publication
(10) Pub. No.: US 2025/0305043 A1
(43) Pub. Date: Oct. 2, 2025

(54) METHOD

(71) Applicant: Oxford Nanopore Technologies PLC, Oxford (GB)

(72) Inventors: Mark John Bruce, Oxford (GB); James Edward Graham, Oxford (GB); Etienne Raimondeau, Oxford (GB); Christopher Peter Youd, Oxford (GB)

(73) Assignee: Oxford Nanopore Technologies PLC, Oxford (GB)

(21) Appl. No.: 18/722,827

(22) PCT Filed: Dec. 22, 2022
(86) PCT No.: PCT/GB2022/053375

§ 371 Date: Jun. 21, 2024

(30) Foreign Application Priority Data
Dec. 23, 2021 (GB) 2118906.3

(51) Int. Cl. C12Q 1/6874

(52) U.S. Cl. CPC C12Q 1/6874

(57) ABSTRACT

Provided herein is a method of characterising a target polynucleotide as it moves with respect to a nanopore using a motor protein. Also provided are polynucleotide adapters and kits comprising such adapters. The methods, kits and adapters find use in characterising polynucleotides, for example in sequencing.

### RELATED APPLICATIONS

This application is a national stage filing under 35 U.S.C. § 371 of international application number PCT/GB2022/053375, filed Dec. 22, 2022, which claims the benefit of United Kingdom application number GB 2118906.3, filed Dec. 23, 2021, each of which is herein incorporated by reference in its entirety.

### REFERENCE TO AN ELECTRONIC SEQUENCE LISTING

The contents of the electronic sequence listing (0036670154US00-SUBSEQ-KZM.xml; Size: 109,243 bytes; and Date of Creation: Dec. 27, 2024) is herein incorporated by reference in its entirety.

### FIELD

The present disclosure provides methods of characterising a target polynucleotide as it moves with respect to a detector such as a transmembrane nanopore. The disclosure also provides novel polynucleotide adapters and kits for use in such methods. The disclosure also provides methods of re-reading a polynucleotide.

### BACKGROUND

Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore.

Polynucleotides are important analytes for sensing in this manner. Nanopore sensing of polynucleotide analytes can reveal the identity and perform single molecule counting of the sensed analytes, but can also provide information on their composition such as their nucleotide sequence, as well as the presence of characteristics such as base modifications, oxidation, reduction, decarboxylation, deamination and more. Nanopore sensing has the potential to allow rapid and cheap polynucleotide sequencing, providing single molecule sequence reads of polynucleotides of tens to tens of thousands bases length.

Two of the essential components of polymer characterization using nanopore sensing are (1) the control of polymer movement through the pore and (2) the discrimination of the component building blocks as the polymer is moved through the pore. During nanopore sensing of analytes such as polynucleotides, it is important to control the movement of the polynucleotide with respect to the pore. Uncontrolled movement can prevent or impede accurate characterisation of the polynucleotides. For example, accurately distinguishing each nucleotide in a homopolymeric polynucleotide is problematic when the movement of the polynucleotide with respect to the pore is not controlled.

It is known to control the movement of a polynucleotide with respect to a detector such as a nanopore by using a motor protein to control the movement of the polynucleotide. Suitable motor proteins include polynucleotide-handling enzymes such as helicases, exonucleases, topoisomerases and the like. The motor protein processes the polynucleotide in a controlled manner. The motor protein can thus be used to control the movement of a polymer such as a polynucleotide with respect to a detector such as a nanopore.

When the detector is a nanopore, disclosed methods typically involve using the motor protein to feed the polynucleotide into the nanopore. This movement direction is described in more detail herein. Methods which involve feeding the polynucleotide into a nanopore have been extensively developed and proven to be very useful in characterising polynucleotides.

However, there remains a need for further methods of characterising polynucleotides. One issue is that in some cases it can be desirable to obtain data different to that obtained from methods which involve feeding polynucleotides into a detector such as a nanopore. For example, the error profiles of data arising from polynucleotide characterisation in methods which involve feeding polynucleotides into a detector can in some circumstances be suboptimal for the accurate characterisation of the polynucleotide. Another issue is that when a motor protein is used to feed a polynucleotide into a detector such as a nanopore, the motor protein may skip forwards in an uncontrolled manner on the polynucleotide strand. This phenomenon is also known as slippage. Slippage can be problematic when characterising polynucleotides as, for example, it can result in one or more nucleotides in the polynucleotide not being accurately characterised. This is particularly problematic when the characterisation of the polynucleotide is to determine its sequence. Strategies for decreasing slippage have to date focused on modifying the motor protein to minimise its propensity to slip on polynucleotide strands. However, alternative methods of moving polynucleotides with respect to detectors such as nanopores which may decrease slippage would also be useful.

There also remains a need for ways of improving the data obtained when characterising polynucleotides. One issue is that in some cases it is desirable to improve the accuracy of the characterisation data obtained when characterising a polynucleotide. In some known methods, a plurality of polynucleotides from a sample of polynucleotides is characterised and the data obtained is aggregated, to improve the overall accuracy. However, this can cause problems. For example, heterogeneity in the sample can mean that when aggregating data obtained from characterising multiple polynucleotide strands, useful information regarding differences between strands can be lost. Furthermore, inefficiencies can arise due to the need to capture a new strand for characterisation once an initial strand has been processed. Alternative and/or improved methods of characterising polynucleotides are thus required.

For these and other reasons there is a need for new and/or improved methods of moving polynucleotides with respect to detectors such as nanopores.

### SUMMARY

The disclosure relates to a method of characterising a target polynucleotide as it moves with respect to a detector having a first opening and a second opening or being comprised in a structure having a first opening and a second opening, by using a motor protein. More particularly, the disclosure relates to methods in which the motor protein controls the movement of the polynucleotide in the direction from the second opening to the first opening. As described in more detail herein, this direction is typically “out” of the detector from the “viewpoint” of the motor protein. The direction of movement of the polynucleotide is thus opposite to known methods in which the polynucleotide is moved into a detector such as a nanopore. This is described in more detail herein.

In the disclosed methods, the motor protein is initially bound to a leader attached to the target polynucleotide. The motor protein may be stalled on the leader at a stalling moiety as described herein, and the methods provided herein may involve destalling the motor protein so that the motor protein can control the movement of the polynucleotide out of the detector (e.g. the nanopore). Methods of stalling and destalling the motor protein are described in more detail herein.

Whilst the disclosure provides nanopores as exemplary detectors, the methods provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore. The disclosed methods are particularly amenable to methods in which a polynucleotide is moved through a detector or through a structure containing a detector, e.g. a well in a detector chip.

Accordingly, provided herein is a method of characterising a target polynucleotide having a leader attached thereto, the method comprising:
(i) contacting a detector having a first opening and a second opening or being comprised in a structure having a first opening and a second opening with the leader, under conditions such that the first opening is contacted with the leader and the target polynucleotide moves in a direction from the first opening to the second opening;
the leader having a motor protein bound thereto at a polynucleotide-binding site of the motor protein;
(ii) taking one or more measurements characteristic of the target polynucleotide as the motor protein controls the movement of the target polynucleotide in a first direction with respect to the detector; wherein the first direction is from the second opening to the first opening;
(iii) unbinding the target polynucleotide from the polynucleotide binding site of the motor protein, such that the target polynucleotide moves in a second direction with respect to the detector; wherein the second direction is from the first opening to the second opening;
(iv) re-binding the target polynucleotide to the polynucleotide binding site of the motor protein; and taking one or more measurements characteristic of the target polynucleotide as the motor protein controls the movement of the target polynucleotide in the first direction with respect to the detector;
thereby characterising the target polynucleotide.

In some embodiments, the target polynucleotide has a first end and a second end and the leader is attached at the first end; and the motor protein is oriented in an orientation to process the target polynucleotide in the direction from the second end towards the first end

In some embodiments, the method comprises repeating steps (iii) and (iv) multiple times.

In some embodiments, prior to step (i) the target polynucleotide is comprised in or consists of a first strand of a double-stranded polynucleotide comprising said first strand and a second strand. In some embodiments, the portion of the first strand between the motor protein and the second end is hybridised to the second strand.

In some embodiments, movement of the target polynucleotide in the direction from the first opening to the second opening comprises separation of the first strand from the second strand. In some embodiments, movement of the target polynucleotide in the direction from the second opening to the first opening comprises annealing of the first strand to the second strand.

In some embodiments, the first strand of the double-stranded polynucleotide is attached to the second strand of the double-stranded polynucleotide.

In some embodiments, in step (ii) the motor protein controls the movement of a first portion of the target polynucleotide in the first direction with respect to the detector; and in step (iv) the motor protein controls the movement of a second portion of the target polynucleotide in the first direction with respect to the detector; and wherein the first portion at least partially overlaps with the second portion. In some embodiments, the first portion is the same as the second portion.

In some embodiments, in step (ii) the motor protein controls the movement of a first portion of the target polynucleotide in the first direction with respect to the detector; and in step (iv) the motor protein controls the movement of a second portion of the target polynucleotide in the first direction with respect to the detector; and wherein the first portion does not overlap with the second portion.

In some embodiments, the distance the target polynucleotide moves with respect to the detector in step (iii) is greater than the distance that the polynucleotide moves with respect to the detector in step (ii) and/or step (iv).

In some embodiments, (a) in step (iii) the distance the target polynucleotide moves with respect to the detector is at least 1000 nucleotides in length and/or (b) in steps (ii) and/or (iv) the distance the target polynucleotide moves with respect to the detector are each independently at least 100 nucleotides in length.

In some embodiments, the second end of the target polynucleotide comprises a blocking moiety to prevent the motor protein from disengaging from the polynucleotide. In some embodiments, the blocking moiety limits the movement of the target polynucleotide through the polynucleotide binding site of the motor protein and thereby limits the movement of the target polynucleotide in the second direction with respect to the detector.

In some embodiments, the detector comprises a transmembrane nanopore spanning a membrane having a cis side and a trans side, and the first opening of the nanopore is at the cis side of the membrane and the second opening of the nanopore is at the trans side; the motor protein controls the movement of the target polynucleotide through the nanopore from the trans side to the cis side of the membrane; and when the target polynucleotide is unbound from the polynucleotide binding site of the motor protein, the target polynucleotide moves through the nanopore from the cis side to the trans side of the membrane.

In some embodiments, the detector comprises a transmembrane nanopore spanning a membrane having a cis side and a trans side, and the first opening of the nanopore is at the trans side of the membrane and the second opening of the nanopore is at the cis side; the motor protein controls the movement of the target polynucleotide through the nanopore from the cis side to the trans side of the membrane; and when the target polynucleotide is unbound from the polynucleotide binding site of the motor protein, the target polynucleotide moves through the nanopore from the trans side to the cis side of the membrane.

In some embodiments, the target polynucleotide does not disengage from the motor protein. In some embodiments, the motor protein is modified to prevent the target polynucleotide disengaging from the motor protein.

In some embodiments, the motor protein is modified to promote unbinding of the target polynucleotide from the polynucleotide binding site of the motor protein and/or to retard re-binding of the target polynucleotide to the polynucleotide binding site of the motor protein.

In some embodiments, the motor protein is modified with a closing moiety for (i) topologically closing the polynucleotide binding site of the motor protein around the target polynucleotide and/or (ii) promoting unbinding of the target polynucleotide from the polynucleotide binding site of the motor protein and/or retarding re-binding of the target polynucleotide to the polynucleotide binding site of the motor protein.

In some embodiments, the motor protein is modified to facilitate attachment of the closing moiety to the motor protein.

In some embodiments, the motor protein is modified by substituting at least one amino acid in the motor protein for cysteine or for a non-natural amino acid.

In some embodiments, the closing moiety comprises a bifunctional crosslinker. In some embodiments, the closing moiety crosslinks two amino acid residues of the motor protein, wherein at least one amino acid crosslinked by the closing moiety is a cysteine or a non-natural amino acid.

In some embodiments, the closing moiety has a length of from about 1 Å to about 100 Å. In some embodiments, the closing moiety has a length of from about 5 Å to about 50 Å.

In some embodiments, the closing moiety comprises a bond. In some embodiments, the closing moiety comprises a disulphide bond.

In some embodiments, the closing moiety comprises a structure of formula [A-B-C], wherein A and C are each independently reactive functional groups for reacting with amino acid residues in the motor protein and B is a linking moiety. In some embodiments, A and C are each independently a cysteine-reactive functional group. In some embodiments, linking moiety B comprises a linear or branched, unsubstituted or substituted alkylene, alkenylene, alkynylene, arylene, heteroarylene, carbocyclylene or heterocyclylene moiety, which moiety is optionally interrupted by and/or terminated in one or more atoms or groups selected from O, N(R), S, C(O), C(O)NR, C(O)O, unsubstituted or substituted arylene, arylene-alkylene, heteroarylene, heteroarylene-alkylene, carbocyclylene, carbocyclylene-alkylene, heterocyclylene and heterocyclylene-alkylene; wherein R is selected from H, unsubstituted or substituted alkyl, and unsubstituted or substituted aryl. In some embodiments, linking moiety B comprises an alkylene, oxyalkylene or polyoxyalkylene group and/or A and C are each maleimide groups.

In some embodiments, the motor protein is a helicase. In some embodiments, the motor protein is a DNA-dependent ATPase (Dda) helicase.

In some embodiments, prior to step (i) the motor protein is stalled on the leader. In some embodiments, the leader comprises a different type of nucleotide to the target polynucleotide. In some embodiments, the target polynucleotide comprises deoxyribonucleotides (DNA) or ribonucleotides (RNA) and the leader comprises one or more stalling units and/or one or more nucleotides lacking both nucleobase and sugar moieties (spacer moieties), deoxyribonucleotides (DNA), ribonucleotides (RNA), peptide nucleotides (PNA), glycerol nucleotides (GNA), threose nucleotides (TNA), locked nucleotides (LNA), bridged nucleotides (BNA), abasic nucleotides or nucleotides having a modified phosphate linkage.

In some embodiments, the second strand of the double-stranded polynucleotide comprises a membrane anchor or a transmembrane pore anchor.

In some embodiments, the method comprises applying a force across the detector, and wherein the motor protein controls the movement of the target polynucleotide with respect to the detector in the direction opposite to the applied force. In some embodiments, the force comprises a voltage potential applied across the detector.

Also provided herein is a polynucleotide adapter having a first end comprising a leader and a second end comprising an attachment point for attaching to a polynucleotide analyte at a first end of the polynucleotide analyte; wherein said polynucleotide adapter comprises a motor protein stalled thereon in an orientation for processing the adapter in a direction from the second end to the first end.

Also provided herein is a kit, comprising a first adapter as defined herein and a second adapter comprising (i) an attachment point for attaching to a polynucleotide analyte at a second end of the polynucleotide analyte; and (ii) a blocking moiety suitable for preventing the motor protein of the first adapter from disengaging from the polynucleotide analyte when the first adapter is attached to the polynucleotide analyte.

Also provided herein is a system for characterising a target polynucleotide comprising:
one or more polynucleotide adapters as defined herein;
a nanopore for characterising the target polynucleotide as the target polynucleotide moves with respect to the nanopore; and
a motor protein for controlling the movement of the target polynucleotide with respect to the nanopore.

In some embodiments, the motor protein and/or said blocking moiety is as defined in any one of the preceding claims.

### BRIEF DESCRIPTION OF THE FIGURES

FIG. 1. A schematic showing the distinction between (A) the direction of movement of a polynucleotide (PN) out of a nanopore under the control of a motor protein in accordance with the methods provided herein, as opposed to (B) the movement of a polynucleotide into the pore in contrasting methods. Open arrows show direction of translocation of the motor protein (MP) and PN. In both cases the MP is for example a 5′-3′ helicase.

FIG. 2. A non-limiting schematic of an embodiment of the methods provided herein, wherein the target polynucleotide is single-stranded; the target polynucleotide comprises a leader (wavy lines) at the first end of the target polynucleotide; and the motor protein is bound to the leader e.g. stalled at the leader. The leader sequence is captured by the nanopore and the single stranded polynucleotide translocates through the nanopore pushing the motor protein towards the second end of the target polynucleotide. The motor protein then controls the movement of the polynucleotide “out of” the pore. The motor protein is then unbound from the target polynucleotide and the target polynucleotide moves back “into” the pore. The motor protein then re-binds to the target polynucleotide and re-reads (RR) the target polynucleotide.

FIG. 3. A non-limiting schematic of an embodiment of the methods provided herein, wherein the target polynucleotide is double-stranded; the target polynucleotide (PN) comprises a leader (wavy lines) located at the first end of a first strand of the target polynucleotide; and the motor protein (MP) is bound to the leader e.g. by being stalled at a stalling moiety comprised in the leader. The leader is captured by the nanopore and the first strand of the target polynucleotide translocates through the nanopore pushing the motor protein towards the second end of the first strand of the target polynucleotide. The motor protein then controls the movement of the first strand of the polynucleotide “out of” the pore. The motor protein is then unbound from the first strand of the target polynucleotide and the first strand of the target polynucleotide moves back “into” the pore. The motor protein then re-binds to the target polynucleotide and re-reads (RR) the target polynucleotide.

FIG. 4. A non-limiting schematic of an embodiment of the methods provided herein, wherein the target polynucleotide is double-stranded; the target polynucleotide (PN) comprises a leader (wavy lines) located at the first end of a first strand of the target polynucleotide; the motor protein (MP) is bound to the leader e.g. by being stalled at a stalling moiety comprised in the leader; a hairpin adapter connects the second end of the first strand of the target polynucleotide and a first end of the second strand of the target polynucleotide, and a blocking moiety is present at the second end of the second strand. The leader is captured by the nanopore and the first strand of the target polynucleotide, the hairpin adapter and the second strand translocate through the nanopore, pushing the motor protein over the second end of the first strand of the target polynucleotide, the hairpin and towards the second end of the second strand. The motor protein then controls the movement of the second strand, hairpin and first strand of the polynucleotide “out of” the pore. The motor protein is then unbound from the first strand of the target polynucleotide and the first strand of the target polynucleotide, the hairpin and the second strand move back “into” the pore. The blocking moiety prevents the motor protein from disengaging from the target polynucleotide. The motor protein then re-binds to the target polynucleotide and re-reads (RR) the target polynucleotide.

FIG. 5. A non-limiting schematic of an embodiment of the methods provided herein, wherein the target polynucleotide is double-stranded; the target polynucleotide (PN) comprises a leader (wavy lines) located at the first end of a first strand of the target polynucleotide; the motor protein (MP) is bound to the leader e.g. by being stalled at a stalling moiety comprised in the leader; and a blocking moiety is present at the second end of the first strand. As shown the double-stranded polynucleotide is symmetrical with the second strand identical to the first strand but this is not essential in the disclosed methods. The leader is captured by the nanopore and the first strand of the target polynucleotide translocates through the nanopore pushing the motor protein towards the second end of the first strand of the target polynucleotide. The motor protein then controls the movement of the first strand of the polynucleotide “out of” the pore. The motor protein is then unbound from the first strand of the target polynucleotide and the first strand of the target polynucleotide moves back “into” the pore. The blocking moiety prevents the motor protein from disengaging from the first strand of the target polynucleotide. The motor protein then re-binds to the target polynucleotide and re-reads (RR) the target polynucleotide.

FIG. 6.
(a) Experimental schematic of Example 1, showing capture, ‘de-stalling’ and sequencing of both strands of a polynucleotide analyte, with occasional rereading of strands. Vs, sequencing potential; Vu, unblock potential. Polarities of applied potential are shown via arrows. The direction of the applied force is the same as the direction of the arrows.
   (A) Sequencing potential applied (120 mV). Open pore; capture of polynucleotide analyte via 3′ leader. Separation of duplex via nanopore; template and complement strand are translocated into the trans compartment.
   (B) Polynucleotide reaches enzyme stalled at spacer moiety. Enzyme cannot move over spacer moiety.
   (C) Unblock potential applied (variable, 0 mV to −120 mV) such that enzyme is moved away from the nanopore and free to translocate over spacer moiety.
   (D) Sequencing potential applied (120 mV). Polynucleotide translocated by nanopore until enzyme reaches nanopore, whereupon enzyme controls movement of polynucleotide out of the nanopore.
   (E) DNA motor moves over template portion and reaches hairpin.
   (F) DNA motor moves over complement portion; template and complement strands refold in the cis compartment. Motor reaches leader section and idles on nanopore.
State (F) may return to state (E) as the enzyme is pushed from 3′-5′, thus enabling rereading (RR) of the strand.
(G) Unblock potential applied; DNA motor and analyte ejected from nanopore.
(b) Representative current-time trace from Example 1, showing an example in which the polynucleotide enzyme is read twice via the enzyme being pushed backwards from the C3 leader under applied potential. Enzyme-controlled portions (i) and (ii) are shown expanded and the C3 level also identified.
(c) Six representative rereading examples from the experiment described in Example 1. The enzyme-controlled portions were mapped using an HMM model trained using data for the pore and enzyme combination used. Reads in the examples shown mapped at least twice to the same strand of a mixture of seven restriction fragments of bacteriophage lambda DNA.

FIG. 7. A non-limiting example of a system for re-reading a polynucleotide strand in a nanopore using a motor protein.
A: adapter for re-reading of a polynucleotide strand. The adapter comprised three oligonucleotide strands: top strand (a); blocker strand (b); and back-blocker strand (c). When hybridised together, these three strands yielded an adapter with 5′-phosphate and 3′ T overhang competent for ligation to dA-tailed double-stranded DNA. A motor protein (MP) was loaded on the adapter. Top strand (a) contained a C3 spacer section (d), a section complementary to the blocker strand (e); stall section to stall the motor protein (f); and motor loading site (g). Blocker strand (b) contained a region complementary to (e) with stalling moiety, and tether complementarity arm (h). Back-blocker strand (c) contained a region with partial complementarity to the top strand, and an arm that contained a biotin-TEG moiety (i). In the control adapter described in Example 5, this biotin-TEG moiety was not present.
B: Cartoon showing motor protein-controlled movement of a polynucleotide out of a nanopore, with re-reading of the polynucleotide. MP, motor protein; PN, polynucleotide. (a), adapter shown in A ligated to a double-stranded, dA-tailed polynucleotide. The adapter was ligated to both ends of the polynucleotide. (b), the species described in (a) with monovalent traptavidin bound, referred to as the “analyte molecule”. The analyte molecule was added to the cis compartment of a nanopore sequencer and a bias voltage of 180 mV applied across the membrane (trans compartment positive with respect to cis). The cartoon shows the stages of re-reading:
   (i) The analyte molecule was captured in the cis opening of the nanopore via its 3′ end.
   (ii) The blocker strand was stripped away from the analyte molecule and the analyte translocated through the nanopore as far as the stalled motor protein.
   (ii) to (iii) The motor protein was pushed (or “dropped back”, as described in Example X1) in the 3′-5′ direction along the DNA, to either the distal (5′) end, whereupon it encountered bound traptavidin, or to a point in the 5′ direction from the end at which the motor protein was loaded.
   (iii) to (iv) The motor protein controlled movement of the polynucleotide in the 5′-3′ direction out of the nanopore.
   (iv) to (v) The motor protein continued to translocate until it reached its original starting point in (i) and (ii). The motor idled on the C3 spacer section of the leader.
   Then, either:
      (v) to (iii) The motor protein was pushed 3′-5′, per (ii) to (iii), and the cycle repeated, or:
      (v) to (vi) The analyte molecule was ejected from the nanopore.

Arrows show how the motor protein or polynucleotide have moved relative to the previous stage.

FIG. 8. Electrical data related to example 5, with either (A) a control adapter ligated to a 3.6-kb polynucleotide analyte, or (B) an adapter containing a 5′ biotin moiety. Electrical data show distinct current levels for open pore and leader. Upon capture of the analyte from open pore, an increase in current was observed to the leader level, followed by a “dropback” signal (stages (ii) to (iii) as described in FIG. 7, B; asterisked), resulting from the enzyme being pushed 3′-5′ through the polynucleotide strand.

In the example shown in (A), without a 5′ biotin moiety, the motor was pushed back 3′-5′ to some earlier point in the strand, whereupon it rebound, controlled movement of the polynucleotide out of the nanopore, reached the “leader” portion, and then “dropped back” once again to open pore. This second “dropback”, starting after ˜982 seconds, resulted in pushing the motor entirely off the 5′ terminus of the polynucleotide strand, as well as full translocation of the polynucleotide strand through the nanopore. In (B), with a 5′ biotin-traptavidin complex present, instead of pushing the motor off the 5′ end upon “dropback”, the motor controlled movement out of the nanopore a second time.

FIG. 9. Electrical data related to example 6. Data show distinct current levels for open pore and leader. Upon application of the voltage after voltage flick 1, the current level immediately increased to the leader level, indicating a strand had been captured. Following a pause, the enzyme was pushed 3′-5′ through the polynucleotide strand (“drop back”,) whereupon it rebound, and controlled movement of the polynucleotide out of the nanopore until it reached the leader portion. This process was repeated for a total of 14 times, indicated by asterisks. After being read 14 times, the strand was ejected by the reversal of the voltage during voltage flick 2.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L a-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “leader” and “leader sequence” are used interchangeably herein.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, |
| | | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

As used herein, an alkylene group is an unsubstituted or substituted bidentate moiety obtained by removing two hydrogen atoms, either both from the same carbon atom, or one from each of two different carbon atoms, of a hydrocarbon compound which may be aliphatic or alicyclic, and is saturated. The hydrocarbon compound may have from 1 to 20 carbon atoms, in which case the alkylene group is a C1-20 alkylene. It may for instance have from 1 to 10 carbon atoms in which case the alkylene group is C1-10 alkylene. Typically it is C₁₋₆ alkylene, or C₁₋₄ alkylene, for example methylene, ethylene, i-propylene, n-propylene, t-butylene, s-butylene or n-butylene.

An alkenylene group is an unsubstituted or substituted bidentate moiety obtained by removing two hydrogen atoms, either both from the same carbon atom, or one from each of two different carbon atoms, of a hydrocarbon compound which may be aliphatic or alicyclic, and which comprises one or more carbon-carbon double bond. The hydrocarbon compound may have from 2 to 20 carbon atoms, in which case the alkenylene group is a C₂₋₂₀ alkenylene. It may for instance have from 2 to 10 carbon atoms in which case the alkenylene group is C₂₋₁₀ alkenylene. Typically it is C₂₋₆ alkenylene, or C₂₋₄ alkenylene.

An alkynylene group is an unsubstituted or substituted bidentate moiety obtained by removing two hydrogen atoms, either both from the same carbon atom, or one from each of two different carbon atoms, of a hydrocarbon compound which may be aliphatic or alicyclic, and which comprises one or more carbon-carbon triple bond. The hydrocarbon compound may have from 2 to 20 carbon atoms, in which case the alkynylene group is a C₂₋₂₀ alkynylene. It may for instance have from 2 to 10 carbon atoms in which case the alkynylene group is C₂₋₁₀ alkynylene. Typically it is C₂₋₆ alkynylene, or C₂₋₄ alkynylene.

An arylene group is an unsubstituted or substituted monocyclic or fused polycyclic bidentate moiety obtained by removing two hydrogen atoms, one from each of two different aromatic ring atoms of an aromatic compound, which moiety has from 5 to 14 ring atoms (unless otherwise specified). Typically, each ring has from 5 to 7 or from 5 to 6 ring atoms. An arylene group may be unsubstituted or substituted.

A heteroarylene group is a bidentate moiety obtained by removing two hydrogen atoms, one from each of two different ring atoms of a heteroaryl group. A heteroaryl group is a substituted or unsubstituted monocyclic or fused polycyclic (e.g. bicyclic or tricyclic) aromatic group which typically contains from 5 to 14 atoms in the ring portion including at least one heteroatom, for example 1, 2 or 3 heteroatoms, selected from O, S, N, P, Se and Si, more typically from O, S and N. Examples include pyridyl, pyrazinyl, pyrimidinyl, pyridazinyl, furanyl, thienyl, pyrazolidinyl, pyrrolyl, oxadiazolyl, isoxazolyl, thiadiazolyl, thiazolyl, imidazolyl, triazolyl, pyrazolyl, oxazolyl, isothiazolyl, benzofuranyl, isobenzofuranyl, benzothiophenyl, indolyl, indazolyl, carbazolyl, acridinyl, purinyl, cinnolinyl, quinoxalinyl, naphthyridinyl, benzimidazolyl, benzoxazolyl, quinolinyl, quinazolinyl and isoquinolinyl.

A carbocyclylene group, also known as a cycloalkylene group, is a bidentate moiety obtained by removing two hydrogen atoms, one from each of two carbon atoms in a unsubstituted or substituted cyclic alkyl group. Typically, the moiety has from 3 to 10 carbon atoms (unless otherwise specified), including from 3 to 10 ring atoms. Examples include cyclopropane (C3), cyclobutane (C4), cyclopentane (C5), cyclohexane (C6), cycloheptane (C7), methylcyclopropane (C4), dimethylcyclopropane (C5), methylcyclobutane (C5), dimethylcyclobutane (C6), methylcyclopentane (C6), dimethylcyclopentane (C7), methylcyclohexane (C7), dimethylcyclohexane (C8), menthane (C10).

A heterocyclylene moiety is a bidentate moiety obtained by removing two hydrogen atoms from two different ring atoms of a heterocyclyl group. A heterocyclyl group is a unsubstituted or substituted cyclic group which typically contains from 5 to 14 atoms in the ring portion including at least one heteroatom, for example 1, 2 or 3 heteroatoms, selected from O, S, N, P, Se and Si, more typically from O, S and N.

Examples include piperazine, piperidine, morpholine, 1,3-oxazinane, pyrrolidine, imidazolidine, oxazolidine, tetrahydropyrazine, tetrahydropyridine, dihydro-1,4-oxazine, tetrahydropyrimidine, dihydro-1,3-oxazine, dihydropyrrole, dihydroimidazole and dihydrooxazole groups.

An arylene-alkylene group is a group formed by forming a bond between an arylene group and an alkylene group as defined herein. A heteroarylene-alkylene group is a group formed by forming a bond between a heteroarylene group and an alkylene group as defined herein. A carbocyclylene-alkylene group is a group formed by forming a bond between an carbocyclylene group and an alkylene group as defined herein,. A heterocyclylene-alkylene group is a group formed by forming a bond between group a heterocyclylene group and an alkylene group as defined herein.

When a group is described as being substituted, it is typically substituted by one or more such as 1, 2 or 3, typically 1 or 2, usually 1 substituent. Suitable substituents may be independently selected from halogen; —OR′ and —NR′₂ (wherein R′ is typically H or unsubstituted C₁₋₂ alkyl, and unsubstituted C₁ to C₂ alkyl.

### Methods of Characterising Polynucleotides

The disclosure relates to a method of characterising a target polynucleotide as it moves with respect to a detector having a first opening and a second opening or being comprised in a structure having a first opening and a second opening, such as a nanopore. Whilst the disclosure provides nanopores as exemplary detectors, the methods provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore. The disclosed methods are particularly amenable to methods in which a polynucleotide is moved through a detector or through a structure containing a detector, e.g. a well in a detector chip.

Any suitable polynucleotide can be characterised using the methods disclosed herein. Polynucleotides which can be characterised in accordance with the disclosed methods are described in more detail herein.

The movement of the target polynucleotide is controlled by using a motor protein. Any suitable motor protein can be used in the methods provided herein. Exemplary motor proteins are described in more detail herein. The methods involve re-reading the polynucleotide, e.g. as it moves back and forth with respect to the detector. The disclosed methods therefore comprise “flossing” the target polynucleotide with respect to the detector. This is described in more detail herein.

In the disclosed methods, the target polynucleotide has a leader attached thereto. Leaders are described in more detail herein.

Initially, in the disclosed methods, the motor protein is bound to the leader at the polynucleotide-binding site of the motor protein. This is described in more detail herein. The motor protein may be stalled on the leader at a stalling moiety. Suitable stalling moieties are described in more detail herein. The stalling of the motor protein on the polynucleotide has various advantages. For example, whilst stalled the motor protein typically consumes less fuel than when unstalled, e.g. when free to move with respect to the polynucleotide. The reduction of this unproductive fuel usage can be advantageous.

The methods provided herein typically involve destalling the motor protein so that the motor protein can control the movement of the polynucleotide with respect to the detector as described in more detail herein. Methods of destalling the motor protein are described in more detail herein. The controlled destalling of the motor protein has various advantages including that the point at which the motor protein starts processing the polynucleotide can be accurately determined. This can be useful in characterising the polynucleotide so that for example no data is lost as a result of undesired movement of the motor protein on the polynucleotide prior to the start of data recordal.

The motor protein is used to control the movement of the target polynucleotide in a first direction with respect to the detector, wherein the first direction is in the direction from the second opening of the detector to the first opening of the detector. Methods characteristic of the target polynucleotide are taken as the target polynucleotide moves in the first direction with respect to the detector.

The target polynucleotide is then unbound from the polynucleotide binding site of the motor protein. This is described in more detail below. Once the target polynucleotide is unbound from the polynucleotide binding site of the motor protein, the target polynucleotide moves in a second direction with respect to the detector.

As discussed herein the second direction is opposite to the first direction. For example, in embodiments wherein the detector is a nanopore, the first direction may be “out” of the nanopore (from the “viewpoint” of the motor protein) and the second direction may be “into” the nanopore (from the “viewpoint” of the motor protein). This is described in more detail herein.

The target polynucleotide is rebound to the polynucleotide binding site of the motor protein. The target polynucleotide is rebound to the same motor protein. In other words, the target polynucleotide is rebound to the same molecule of the motor protein, not merely to a different molecule of the same type of motor protein. The polynucleotide-handling protein is again used to control the movement of the target polynucleotide in the first direction with respect to the detector. Further measurements characteristic of the target polynucleotide are taken as the conjugate moves in the first direction with respect to the detector. The first direction is the same as the first direction described above.

Accordingly, disclosed herein is a method of characterising a target polynucleotide having a leader attached thereto, the method comprising:
(i) contacting a detector having a first opening and a second opening or being comprised in a structure having a first opening and a second opening with the leader, under conditions such that the first opening is contacted with the leader and the target polynucleotide moves in a direction from the first opening to the second opening;
   the leader having a motor protein bound thereto at a polynucleotide-binding site of the motor protein;
(ii) taking one or more measurements characteristic of the target polynucleotide as the motor protein controls the movement of the target polynucleotide in a first direction with respect to the detector; wherein the first direction is from the second opening to the first opening;
(iii) unbinding the target polynucleotide from the polynucleotide binding site of the motor protein, such that the target polynucleotide moves in a second direction with respect to the detector; wherein the second direction is from the first opening to the second opening;
(iv) re-binding the target polynucleotide to the polynucleotide binding site of the motor protein; and taking one or more measurements characteristic of the target polynucleotide as the motor protein controls the movement of the target polynucleotide in the first direction with respect to the detector;
   thereby characterising the target polynucleotide. Characterising the target polynucleotide may for example comprise determining the sequence of the target polynucleotide.

In some embodiments steps (iii) and (iv) of the disclosed methods are repeated multiple times by sequentially allowing the motor protein to bind and rebind to the target polynucleotide. In such a manner, the target polynucleotide may oscillate with respect to the detector (i.e. it may be “flossed” with respect to the first and second openings of the detector). This “flossing” allows the target polynucleotide to be repeatedly characterised. In some embodiments this allows the accuracy of the characterisation information to be increased.

The disclosed methods are based at least in part on the recognition that the data obtained when a polynucleotide is moved out of a detector such as a nanopore can vary from that obtained when the same polynucleotide is moved into the detector (e.g. the nanopore). The data characteristics including signal profile, noise profile, and error profile, can in some embodiments all differ from contrasting methods in which the same polynucleotide is moved into a detector such as a nanopore. In some embodiments the data obtained in the disclosed methods has advantages compared to data obtained in other known methods. The disclosed methods thus increase the options available when polynucleotide characterisation is needed. Users desiring to characterise polynucleotides can thus choose the method best suited to the specific application at issue. The measurements taken as the target polynucleotide moves in the first direction can in some embodiments be combined or compared in order to improve the characterisation of the polypeptide.

The disclosed methods have many advantages compared to previously known methods. For example, each reading of the target polynucleotide should be of equivalent accuracy as the same strand and same detector moiety is used. This allows for the same basecalling model to be used for each read. It also facilitates combining data from multiple reads. Furthermore, the native sequence is re-read multiple times, allowing (for example) epigenetic information to be retained. The methods are also adaptive: re-reading can be repeated multiple times until data of required accuracy has been obtained.

### Characterising a Target Polynucleotide

As discussed above, the target polynucleotide is characterised using a detector.

The target polynucleotide interacts with the detector. For example, the target polynucleotide may thread through the first and second openings of the detector. A leader is attached to the target polynucleotide. The leader may facilitate the threading of the target polynucleotide through the first and second openings. As the target polynucleotide moves with respect to the detector it is characterised.

In some embodiments the target polynucleotide has a first end and a second end and the leader is attached at the first end; and the motor protein is oriented in an orientation to process the target polynucleotide in the direction from the second end towards the first end. Thus, the motor protein moves in the direction towards the second end of the polynucleotide with respect to the polynucleotide and the polynucleotide moves in the direction from the second end towards the first end with respect to the motor protein.

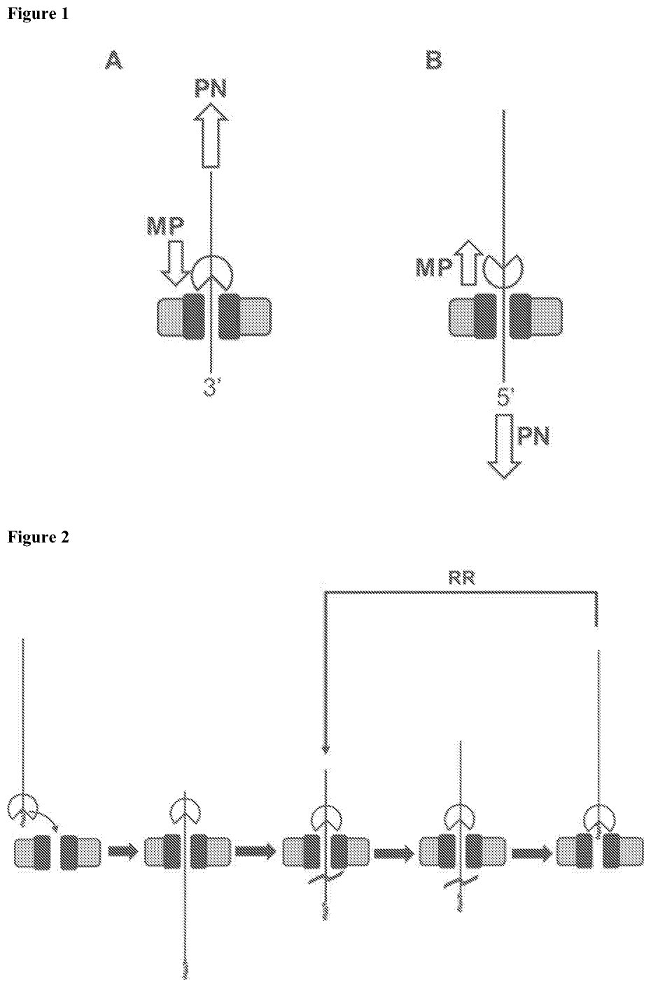

In the example illustrated in FIG. 1 the motor protein (MP) moves the target polynucleotide (PN) in the first direction “out” of the first opening of the detector, from the “viewpoint” of the motor protein. When unbound from the motor protein the target polynucleotide may move in the second direction “into” the first opening of the detector, from the “viewpoint” of the polynucleotide-handling protein. The distinction between the direction of movement of a polynucleotide out of the detector such as a nanopore in methods provided herein, as opposed to the movement of a polynucleotide into the pore in contrasting methods, is illustrated schematically in FIG. 1.

As those skilled in the art will appreciate, the notation “out” relates to the overall movement of the polynucleotide towards the motor protein. This direction of movement may be contrasted with an alternative mode in which the target polynucleotide is moved by the motor protein “into” the first opening of the detector. The difference in these movement schemes is profound. In methods provided herein in which the polynucleotide is moved “out” of the detector (e.g. out of a nanopore), the direction of movement of the target polynucleotide is from the entrance of the detector furthest away from the motor protein (i.e. the distal entrance) towards the entrance of the detector closest to the motor protein (the proximal entrance). In contrasting methods in which the polynucleotide is moved “into” the detector (e.g. into a nanopore), the direction of movement of the target polynucleotide is from the entrance of the nanopore closest to the motor protein (the proximal entrance) towards the entrance of the detector furthest away from the motor protein (the distal entrance).

For example, in some embodiments the detector may be or comprise a nanopore. When the detector is or comprises a nanopore, the first and second openings may be referred to as the cis and trans openings of the nanopore. Often the first opening is the cis opening and the second opening is the trans opening, but in some embodiments the first opening is the trans opening and the second opening is the cis opening, respectively. The notation “cis” and “trans” openings in nanopores is routine in the art. For example, the cis opening of a nanopore typically faces the cis chamber of a nanopore device such as an apparatus as described herein having cis and trans chambers, and the trans opening typically faces the trans chamber.

In some embodiments of the provided methods, therefore, the nanopore spans a membrane having a cis side and a trans side, and the first opening of the nanopore is at the cis side of the membrane and the second opening of the nanopore is at the trans side. In such embodiments, the motor protein is located on the cis side of the membrane and controls the movement of the target polynucleotide through the nanopore in the direction from the trans side to the cis side of the membrane, i.e. in the direction from the trans side to the cis side of the nanopore.

In other embodiments of the provided methods the nanopore spans a membrane having a cis side and a trans side, and the first opening of the nanopore is at the trans side of the membrane and the second opening of the nanopore is at the cis side. In such embodiments, the motor protein is located on the trans side of the membrane and controls the movement of the target polynucleotide through the nanopore in the direction from the cis side to the trans side of the membrane, i.e. in the direction from the cis side to the trans side of the nanopore.

In more detail, the method comprise taking one or more measurements characteristic of the target polynucleotide as a motor protein controls the movement of the target polynucleotide in a first direction with respect to the detector. The first direction may be the direction in which the motor protein drives the movement of the polynucleotide. The first direction may be in the direction of a force applied across the detector. The first direction may be in the opposite direction to that of a force applied across the detector.

Often, the detector is comprised in a structure having a first opening and a second opening, or comprises a transmembrane nanopore having a first opening and a second opening; and step (i) comprises contracting the first opening with the leader attached to the target polynucleotide. Typically, the motor protein controls the movement of the target polynucleotide in the direction from the second opening to the first opening. Typically, when the target polynucleotide is unbound from the polynucleotide binding site of the motor protein, the target polynucleotide moves in the direction from the first opening to the second opening.

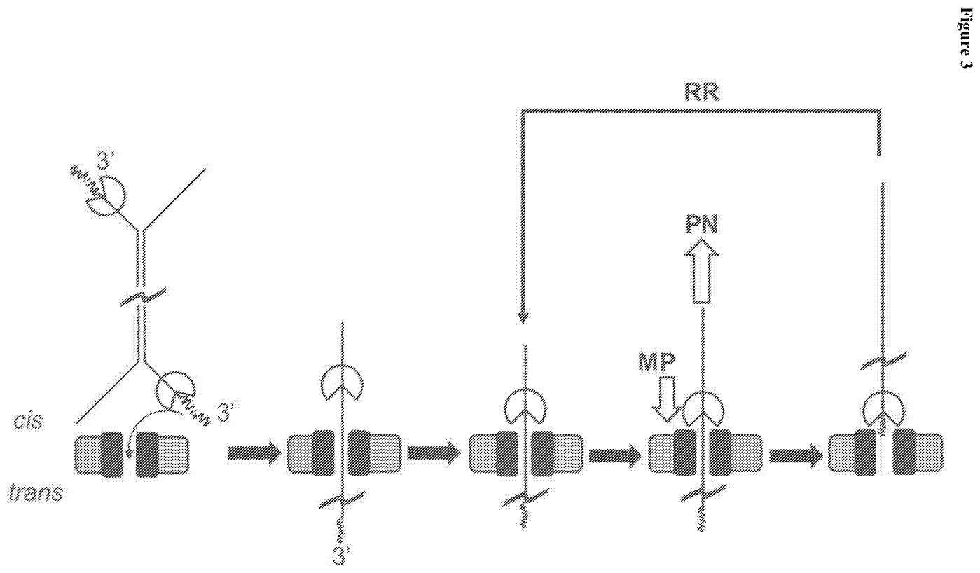

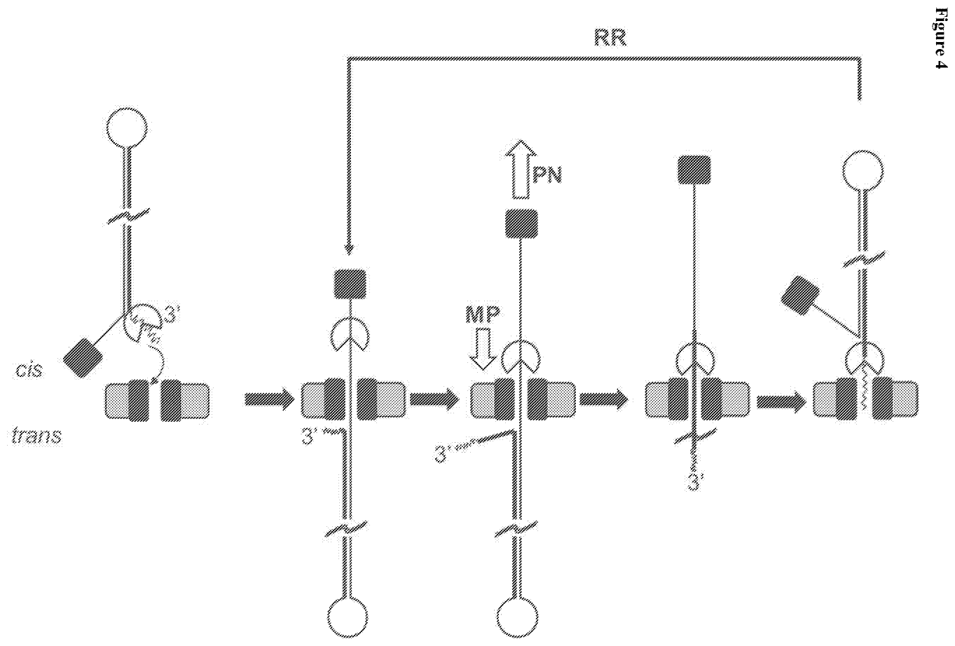

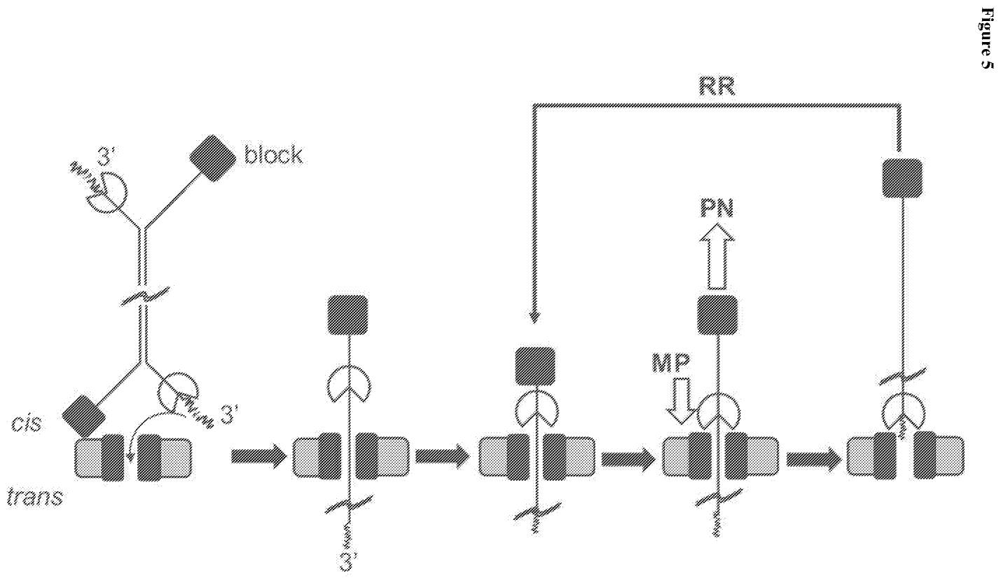

Usually, when the detector is or comprises a nanopore, the first direction is “out” of the nanopore as described herein. Thus, in some embodiments the movement of the polynucleotide whilst one or more measurements are taken is out of the nanopore. In some embodiments the nanopore spans a membrane having a cis side and a trans side; the first opening of the nanopore is at the cis side of the membrane and the second opening of the nanopore is at the trans side; the motor protein is located on the cis side of the membrane and controls the movement of the target polynucleotide through the nanopore from the trans side to the cis side of the membrane i.e. from the trans side to the cis side of the nanopore. In such embodiments the motor protein controls the movement of the target polynucleotide through the nanopore in the direction “out” of the nanopore (from the “viewpoint” of the motor protein). When unbound from the motor protein the target polynucleotide moves in the second direction with respect to the detector. The movement of the target polynucleotide in the second direction is thus movement of the polynucleotide through the nanopore in the direction “into” the nanopore (from the “viewpoint” of the motor protein); i.e. in the direction from the cis side to the trans side of the membrane; i.e. from the cis side to the trans side of the nanopore. This is depicted in FIG. 2.

Of course the opposite setup can also be used in the methods disclosed herein. For example, as shown the detector may comprise a transmembrane nanopore spanning a membrane having a cis side and a trans side. In some embodiments, the first opening of the nanopore is at the trans side of the membrane and the second opening of the nanopore is at the cis side; the motor protein is located on the trans side of the membrane and controls the movement of the target polynucleotide through the nanopore from the cis side to the trans side of the membrane i.e. from the cis side to the trans side of the nanopore. In such embodiments the motor protein controls the movement of the target polynucleotide through the nanopore in the direction “out” of the nanopore (from the “viewpoint” of the motor protein). When unbound from the motor protein the target polynucleotide moves in the second direction with respect to the detector. The movement of the target polynucleotide in the second direction is thus movement of the polynucleotide through the nanopore in the direction “into” the nanopore (from the “viewpoint” of the motor protein); i.e. in the direction from the trans side to the cis side of the membrane; i.e. from the trans side to the cis side of the nanopore.

In other embodiments the first direction may be “into” the detector e.g. a nanopore as described herein. Thus, in some embodiments the detector may comprise a transmembrane nanopore spanning a membrane having a cis side and a trans side and the movement of the polynucleotide in the first direction is into the nanopore. In some embodiments the nanopore spans a membrane having a cis side and a trans side; the first opening of the nanopore is at the cis side of the membrane and the second opening of the nanopore is at the trans side; the motor protein is located on the cis side of the membrane and controls the movement of the target polynucleotide through the nanopore from the cis side to the trans side of the membrane i.e. from the cis side to the trans side of the nanopore. In such embodiments the motor protein controls the movement of the target polynucleotide through the nanopore in the direction “into” the nanopore (from the “viewpoint” of the motor protein). When unbound from the motor protein the target polynucleotide moves in the second direction with respect to the detector. The movement of the target polynucleotide in the second direction is thus movement of the polynucleotide through the nanopore in the direction “out of” the nanopore (from the “viewpoint” of the motor protein); i.e. in the direction from the trans side to the cis side of the membrane; i.e. from the trans side to the cis side of the nanopore. In other embodiments, the nanopore spans a membrane having a cis side and a trans side; the first opening of the nanopore is at the trans side of the membrane and the second opening of the nanopore is at the cis side; the motor protein is located on the trans side of the membrane and controls the movement of the target polynucleotide through the nanopore from the trans side to the cis side of the membrane i.e. from the trans side to the cis side of the nanopore. In such embodiments the motor protein controls the movement of the target polynucleotide through the nanopore in the direction “into” of the nanopore (from the “viewpoint” of the motor protein). When unbound from the motor protein the target polynucleotide moves in the second direction with respect to the detector. The movement of the target polynucleotide in the second direction is thus movement of the polynucleotide through the nanopore in the direction “out of” the nanopore (from the “viewpoint” of the motor protein); i.e. in the direction from the cis side to the trans side of the membrane; i.e. from the cis side to the trans side of the nanopore.

Usually the first direction of the movement of the polynucleotide with respect to the first and second openings of the detector is “out” of the first opening, i.e in the direction from the second opening to the first opening.

In some embodiments the method comprises applying a force (e.g. a voltage potential) across the first and second openings of the detector e.g. across a nanopore, and the motor protein controls the movement of the target polynucleotide through the nanopore in the direction opposite to the applied force.

It is important to distinguish the movement of the polynucleotide in the second direction with respect to the detector from spontaneous slipping that may occur. A slip of one or two bases, for example, is not an example of re-reading as described herein. Typically, in in step (iii) the distance the target polynucleotide moves with respect to the detector is at least 10 nucleotides in length. In some embodiments the distance the target polynucleotide moves with respect to the detector is at least 20 nucleotides in length, e.g. at least 30 nucleotides in length, such as at least 40 nucleotides in length, e.g. at least 50 nucleotides in length, such as at least 100 nucleotides in length. Longer distances may be used. In some embodiments in step (iii) the distance the target polynucleotide moves with respect to the detector is at least 1000 nucleotides (1 kb) in length, such as at least 2 kb, e.g. at least 5 kb or at least 10 kb in length, e.g. at least 100 kb or at least 1000 kb in length.

Steps (iii) and (iv) of the method may be repeated multiple times in order to re-read the target polynucleotide multiple times. Steps (iii) and (iv) may be repeated at least once, such as at least 2 times, such as at least 3 times, e.g. at least 4 times, for example at least 5 times, e.g. at least 10 times, such as at least 20 times, for example at least 50 times, such as at least 100 times, e.g. at least 1000 times, such as at least 10,000 times, e.g. at least 100,000 times or more. Thus, the method may comprise “flossing” the polynucleotide back and forward with respect to the detector.

Thus, if steps (iii) and (iv) are repeated 1 time (and only 1 time) so that the method comprises steps (iii) and (iv) twice and only twice, the method will comprise steps (i), (ii), (iii), (iv), (iii₁), and (iv₁), and measurements characteristic of three portions of the polynucleotide will be taken: a first portion in step (ii); a second portion in step (iii) and (iv); and a third portion in steps (iii₁) and (iv₁). If steps (iii) and (iv) are repeated 2 times (and only 2 times) so that the method comprises steps (iii) and (iv) three times and only three times, the method will comprise steps (i), (ii), (iii), (iv), (iii₁), (iv₁), (iii₂) and (iv₂); and measurements characteristic of four portions of the polynucleotide will be taken: a first portion in step (ii); a second portion in steps (iii) and (iv); a third portion in steps (iii₁) and (iv₁) and a fourth portion in steps (iii₂) and (iv₂). In other words, if steps (iii) and (iv) are repeated n times, then each repeat leads to measurements characteristic of (n+2) portions of the polynucleotide being taken. Repeating steps (iii) and (iv) multiple times can lead to improved characterisation, because the portion of the polynucleotide that is being interrogated by the nanopore is sampled multiple times, and thus any stochastic errors that may be recorded in the analysis become less statistically significant. The accuracy of the characterising data thus obtained can therefore be improved. The methods allow very high accuracy levels to be reached, for example at least 99% accuracy, at least 99.9% accuracy, or at least 99.99% accuracy. Thus, in some embodiments steps (iii) and (iv) are repeated until an accuracy level of at least 99%, such as at least 99.9%, or at least 99.99% accuracy has been reached.

The portion of the polynucleotide which is read in step (ii) and the portion of the polynucleotide which is read in step (iv) of the methods typically overlap. In other words, the method involves re-reading at least a part of the polynucleotide multiple times. Thus, in some embodiments in step (ii) the motor protein controls the movement of a first portion of the target polynucleotide in the first direction with respect to the detector; and in step (iv) the motor protein controls the movement of a second portion of the target polynucleotide in the first direction with respect to the detector; and the first portion at least partially overlaps with the second portion. In some embodiments the second portion overlaps with at least 10%, at least 20%, at least 30%, at least 40%, at least 50%, at least 60%, at least 70%, at least 80%, at least 90%, at least 95%, at least 97%, at least 98% or at least 99% of the first portion. In some embodiments the first portion is the same as the second portion. Thus in some embodiments a portion of the polynucleotide is repeatedly characterised in the provided methods. When at each repeat the second portion of the polynucleotide partially but not wholly overlaps with the first portion of the polynucleotide of the preceding repeat the polynucleotide is ratcheted in a zig-zag manner with respect to the detector. When at each repeat the second portion of the polynucleotide wholly overlaps with the first portion of the polynucleotide of the preceding repeat the same portion of the polynucleotide is flossed back and forwards with respect to the detector.

However, in some embodiments the portion of the polynucleotide which is read in step (ii) and the portion of the polynucleotide which is read in step (iv) of the methods do not overlap. In other words, the method involves re-reading at least a part of the polynucleotide multiple times. Thus, in some embodiments in step (ii) the motor protein controls the movement of a first portion of the target polynucleotide in the first direction with respect to the detector; and in step (iv) the motor protein controls the movement of a second portion of the target polynucleotide in the first direction with respect to the detector; and the first portion does not overlap with the second portion.

In some embodiments the distance that the target polynucleotide initially moves with respect to the detector in step (i) is at least 1000 nucleotides in length. In some embodiments the distance that the target polynucleotide moves with respect to the detector in step (i) is at least 2000 nucleotides in length, e.g. at least 5000 nucleotides in length, such as at least 10,000 nucleotides in length e.g. at least 15000 nucleotides in length such as at least 20,000 nucleotides in length, such as at least 25,000 nucleotides in length, e.g. at least 30,000 nucleotides in length, e.g. at least 50,000 nucleotides in length or more, such as at least 100,000 nucleotides in length, or even more. This increase in initial “drop back” is a particular advantage of the disclosed methods and e.g. may in some embodiments allows for extended initial reads of the target polynucleotide.

In some embodiments the distance the target polynucleotide moves with respect to the detector in step (iii) is greater than the distance that the polynucleotide moves with respect to the detector in step (ii). In some embodiments the distance the target polynucleotide moves with respect to the detector in step (iii) is greater than the distance that the polynucleotide moves with respect to the detector in step (iv). In some embodiments the distance the target polynucleotide moves with respect to the detector in step (iii) is greater than the distance that the polynucleotide moves with respect to the detector in step (ii) and/or step (iv).

In some embodiments the distance that the target polynucleotide moves with respect to the detector in step (iii) is at least 1000 nucleotides in length. In some embodiments the distance that the target polynucleotide moves with respect to the detector in step (iii) is at least 2000 nucleotides in length, e.g. at least 5000 nucleotides in length, such as at least 10,000 nucleotides in length e.g. at least 15000 nucleotides in length such as at least 20,000 nucleotides in length, such as at least 25,000 nucleotides in length, e.g. at least 30,000 nucleotides in length, e.g. at least 50,000 nucleotides in length or more, such as at least 100,000 nucleotides in length.

In some embodiments the distance the target polynucleotide moves with respect to the detector in steps (ii) and/or (iv) are each independently at least 100 nucleotides in length. In some embodiments the distance the target polynucleotide moves with respect to the detector in steps (ii) and/or (iv) are each independently at least 200 nucleotides in length, e.g. at least 500 nucleotides in length, such as at least 1,000 nucleotides in length e.g. at least 2000 nucleotides in length such as at least 5,000 nucleotides in length e.g. at least 10,000 nucleotides in length.

### Applied Forces During Movement

In some embodiments of the disclosed methods, a force may be applied across the detector, e.g. across the nanopore. The force can be controlled in order to control the methods. For example, by increasing the force the movement of the polynucleotide with respect to the detector (e.g. nanopore) can be increased or decreased, e.g. the rate at which the polynucleotide moves through the pore can be controlled.

In the methods provided herein, any suitable force can be applied. The force may be a potential applied across the first and second openings, e.g. when the detector is or comprises a nanopore the force may be a potential applied across the nanopore. In some embodiments, no external force is applied across the first and second openings of the detector (e.g. when the detector is or comprises a nanopore in some embodiments no force may be applied across the nanopore). For example, in some embodiments no electrical potential is applied. Such embodiments are in some embodiments particularly suited to methods in which optical measurements are taken as the polynucleotide moves with respect to the nanopore.

In other embodiments, the force may be a voltage force applied across the first and second openings, e.g. when the detector is or comprises a nanopore a voltage force may be a potential applied across the nanopore. A voltage force may be applied using any suitable apparatus, such as an apparatus described herein. Suitable voltage potentials are described in more detail herein.

In some embodiments the force is applied across a membrane in which the detector e.g. a nanopore is embedded. The force is typically applied from the cis side to the trans side of the membrane; i.e. from the cis side to the trans side of the nanopore. The force may be a positive voltage applied across the nanopore or a negative voltage applied across the nanopore.

Typically the force is a positive voltage applied across the first and second openings, e.g. when the detector is or comprises a nanopore the force may be a positive voltage across the nanopore such that the trans side of the pore is positive relative to the cis side of the pore. In such embodiments, the force thus attracts the negatively charged polynucleotides to move from the cis side to the trans side of the pore. In such embodiments, the methods provided herein typically comprise using the motor protein at the cis side of the pore to control the movement of the polynucleotide in the direction from the trans side of the pore to the cis side of the pore against the applied force; i.e. in the direction opposite to the applied force. However, in some embodiments, the methods provided herein comprise allowing the polynucleotide to move in the direction from the cis side of the pore to the trans side of the pore in the same direction as the applied force.

In other embodiments the force is a negative voltage applied across the first and second openings, e.g. when the detector is or comprises a nanopore the force may be a negative voltage across the nanopore such that the trans side of the pore is negative relative to the cis side of the pore. In such embodiments, the force thus attracts the negatively charged polynucleotides to move from the trans side to the cis side of the pore. In such embodiments, the methods provided herein typically comprise using the motor protein at the trans side of the pore to control the movement of the polynucleotide in the direction from the cis side of the pore to the trans side of the pore against the applied force; i.e. in the direction opposite to the applied force. However, in some embodiments, the methods provided herein comprise allowing the polynucleotide to move in the direction from the trans side of the pore to the cis side of the pore in the same direction as the applied force.

As explained below, however, the methods provided herein do not rely on moving the polynucleotide in the direction opposite to an applied force. The direction of movement can in some embodiments be in the same direction as any applied force, whilst still being in the direction out of the detector e.g. pore. In such embodiments, the motor protein typically controls the movement of the polynucleotide out of a detector such as a pore at a speed greater than that which would arise from the applied force alone.

Accordingly, in some embodiments the force is a positive voltage applied across the first and second openings, e.g. when the detector is or comprises a nanopore the force may be a positive voltage across the nanopore such that the trans side of the pore is positive relative to the cis side of the pore; and the methods may comprise using the motor protein at the trans side of the pore to control the movement of the polynucleotide in the direction from the cis side of the pore to the trans side of the pore with the applied force. In other embodiments, the force is a negative voltage applied across the first and second openings, e.g. when the detector is or comprises a nanopore the force may be a positive voltage across the nanopore such that the trans side of the pore is negative relative to the cis side of the pore; and the methods may comprise using the motor protein at the cis side of the pore to control the movement of the polynucleotide in the direction from the trans side of the pore to the cis side of the pore with the applied force.

### Setup

In the provided methods, a leader is attached to the target polynucleotide.

In step (i) of the disclosed methods, the leader is contacted with the first opening of the detector. The target polynucleotide then moves in a direction from the first opening to the second opening.

The leader may be captured by the detector (e.g. the nanopore) in the methods provided herein.

In some embodiments the leader is directly attached to the first end of the polynucleotide. In some embodiments the leader is attached to the first end of the polynucleotide by a linker. The leader may be comprised in an adapter attached to the first end of the polynucleotide. Adapters are described in more detail herein.

Any suitable leader may be used, as described in more detail herein. In some embodiments the leader is capable of threading through the first opening of the detector. In some embodiments the leader is capable of threading through the second opening of the detector. In some embodiments the leader is capable of threading through the first opening and the second opening of the detector. In some embodiments the detector is or comprises a nanopore and the leader is capable of threading at least part of the way through the nanopore. In some embodiments the detector is or comprises a nanopore and the leader is capable of translocating the nanopore.

Typically, prior to step (i) of the disclosed methods a leader is provided at a first end of the polynucleotide (e.g. by being comprised in the first end of the target polynucleotide or by being comprised in a polynucleotide adapter attached to the first end of the target polynucleotide) and the motor protein is bound to the leader. For example, the leader may be present at the 3′ end of a single-stranded polynucleotide and the motor protein may be bound to the leader. Alternatively, the leader sequence may be present at the 5′ end of a single-stranded polynucleotide and the motor protein may be bound to the leader. The motor protein may be stalled on the leader. The motor protein may be bound to the leader at a stalling moiety as described in more detail herein.

In some embodiments, the target polynucleotide is single-stranded; the target polynucleotide comprises a leader, wherein the leader is located at the first end of the target polynucleotide or is comprised in an adapter attached to the first end of the target polynucleotide; and the motor protein is bound to e.g. stalled at the leader. In such embodiments, the leader is typically captured by the first opening of the detector (e.g. the first opening of a nanopore) and the leader translocates through the nanopore until the stalled motor protein is reached. The motor protein may be “pushed back” over the target polynucleotide towards the second end of the target polynucleotide. The motor protein controls the movement of the polynucleotide out of the pore. The motor protein is then unbound from the target polynucleotide and the target polynucleotide moves in the second direction with respect to the first and second openings of the detector such as the pore. The second direction may be into the pore. The motor protein then rebinds to the target polynucleotide and controls the movement of the target polynucleotide in the first direction with respect to the detector, thus re-reading the target polynucleotide. This setup is illustrated schematically in FIG. 2.

In some embodiments, the target polynucleotide is double stranded.

In some embodiments, the target polynucleotide is double-stranded and comprises a first strand and a second strand; the target polynucleotide comprises a leader, wherein the leader sequence is located at a first end of the polynucleotide and is comprised in the first strand or attached thereto or is comprised in an adapter attached to the first strand; and the motor protein is bound to the leader. The first strand of the double-stranded polynucleotide may be the template strand. The first strand of the double-stranded polynucleotide may be the complement strand. In some embodiments the motor protein is stalled at the leader attached to the first strand of the target polynucleotide or comprised in an adapter attached to the first strand of the target polynucleotide. In some embodiments the target polynucleotide is double-stranded and comprises a first strand and a second strand; the target polynucleotide comprises a leader sequence, wherein the leader is located at a first end of the polynucleotide and is comprised in or attached to the first strand or is comprised in an adapter attached to the first strand; and the motor protein is stalled at the leader.

For example, the leader sequence may be present at the 3′ end of the first strand of the double-stranded polynucleotide and the motor protein may be bound to e.g. stalled at the leader. Alternatively, the leader sequence may be present at the 5′ end of the first strand of the double-stranded polynucleotide and the motor protein may be bound to e.g. stalled at the leader.

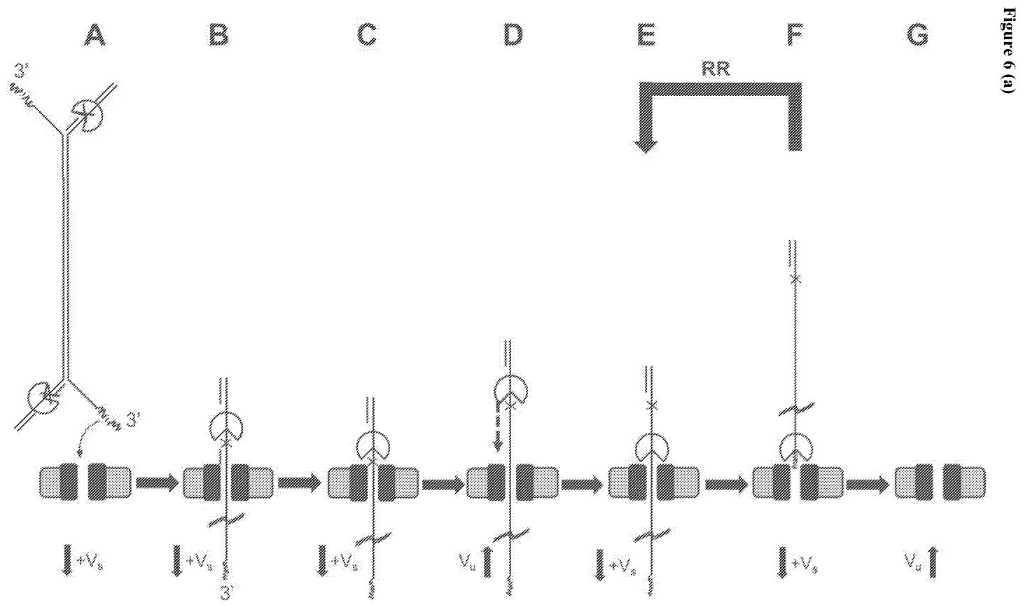

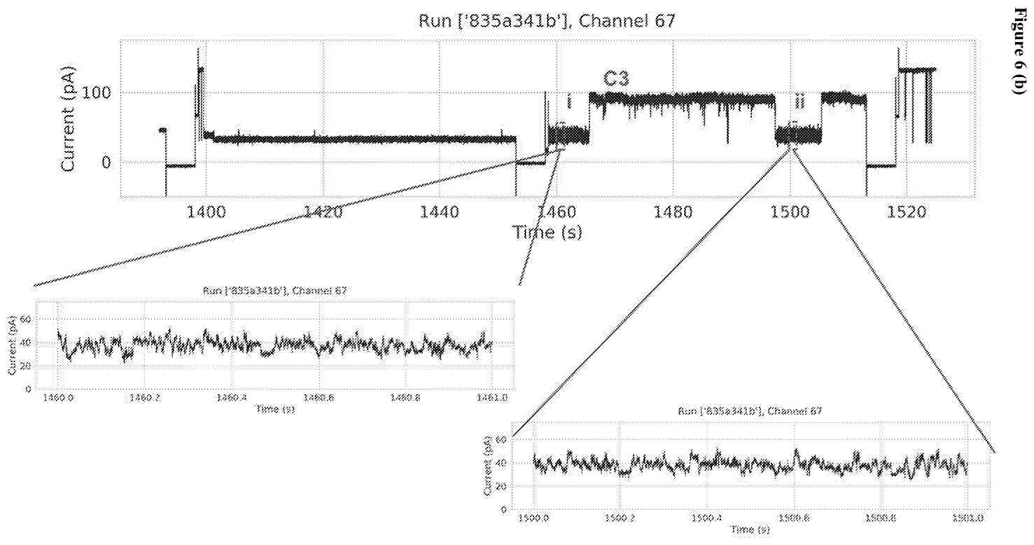

In such embodiments, the leader sequence is typically captured by the nanopore and the first strand translocates through the nanopore until the stalled motor protein is reached. The motor protein may be “pushed back” over the target polynucleotide towards the second end of the first strand of the target polynucleotide. The motor protein controls the movement of the polynucleotide out of the pore. The motor protein is then unbound from the target polynucleotide and the target polynucleotide moves in the second direction with respect to the first and second openings of the detector such as the pore. The second direction may be into the pore. The motor protein then rebinds to the target polynucleotide and controls the movement of the first strand of the target polynucleotide in the first direction with respect to the detector, thus re-reading the target polynucleotide. This setup is illustrated schematically in FIG. 3.

In some embodiments the first strand and the second strand are attached together by a hairpin adapter at the second end of the first strand. In some embodiments the hairpin adapter is attached at its 5′ end to the 3′ end of the first strand and is attached at its 3′ end to the 5′ end of the second strand of the target double-stranded polynucleotide. In some embodiments the hairpin adapter is attached at its 3′ end to the 5′ end of the first strand and is attached at its 5′ end to the 3′ end of the second strand of the target double-stranded polynucleotide. Accordingly, the hairpin adapter connects the first strand to the second strand. The hairpin adapter typically connects the second end of the first strand of the double-stranded polynucleotide to the first end of the second strand of the double-stranded polynucleotide.

In some embodiments the target polynucleotide is double-stranded and comprises a first strand and a second strand; the target polynucleotide comprises a leader, wherein the leader is located at a first end of the polynucleotide and is comprised in the first strand or is comprised in an adapter attached to the first strand; the first strand and the second strand are attached together by a hairpin adapter at the second end of the first strand; and the motor protein is bound to the leader. In some embodiments the motor protein is stalled at the leader attached to the first strand of the target polynucleotide or comprised in an adapter attached to the first strand of the target polynucleotide. In some embodiments the target polynucleotide is double-stranded and comprises a first strand and a second strand; the target polynucleotide comprises a leader sequence, wherein the leader sequence is located at a first end of the polynucleotide and is comprised in the first strand or is comprised in an adapter attached to the first strand; the first strand and the second strand are attached together by a hairpin adapter attached to (i) the second end of the first strand and (ii) a first end of the second strand; and the motor protein is stalled at the leader.

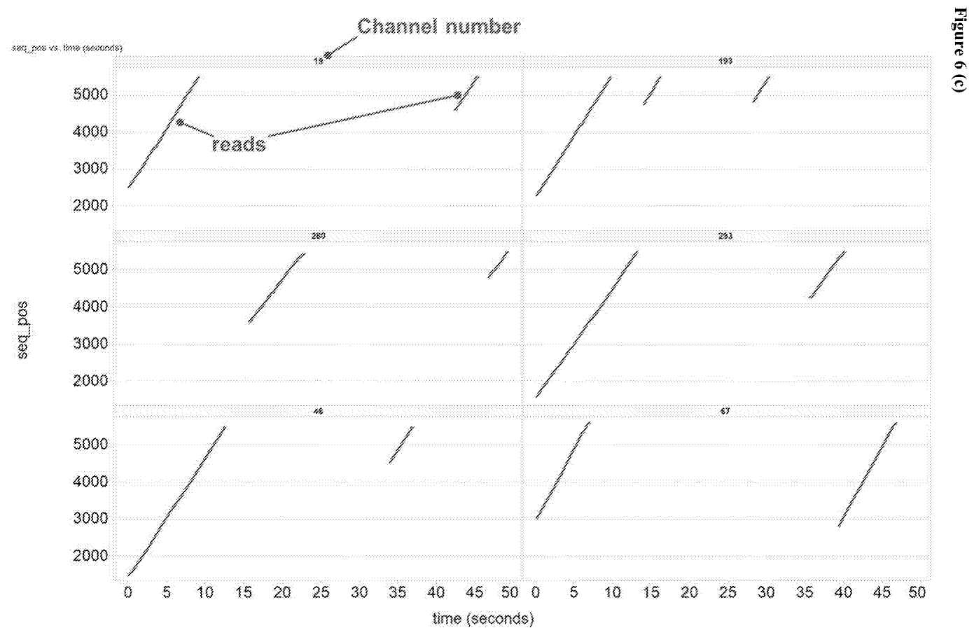

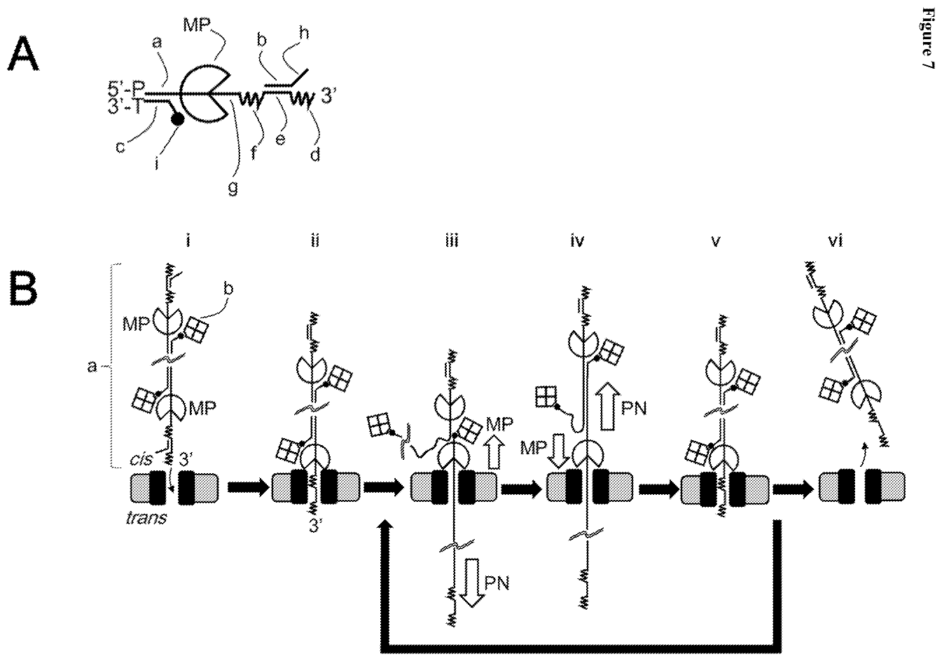

In such embodiments, the leader sequence is typically captured by the nanopore and the double-stranded polynucleotide translocates through the nanopore until the stalled motor protein is reached. The motor protein may be “pushed back” over the first strand of the double-stranded polynucleotide, optionally the hairpin adapter and further optionally the second strand of the double-stranded polynucleotide towards the second end of the first strand of the target polynucleotide. The motor protein controls the movement of the second strand, and optionally also the hairpin adapter and further optionally the first strand of the double-stranded polynucleotide out of the pore. The motor protein is then unbound from the target polynucleotide and the target polynucleotide moves in the second direction with respect to the first and second openings of the detector such as the pore. The second direction may be into the pore. The motor protein then rebinds to the target polynucleotide and controls the movement of the the target polynucleotide in the first direction with respect to the detector, thus re-reading the target polynucleotide. This setup is illustrated schematically in FIG. 4.

The motor protein may be bound to the leader at any point. The motor protein may be bound to the leader at the point where the leader is attached to the first end of the polypeptide. The motor protein may be bound to the free end of the leader. The motor protein may be bound at any point along the leader. The motor protein may be bound to a portion of a polynucleotide such as a portion of DNA or RNA comprised in the leader, wherein the leader comprises a portion of a polynucleotide and a second type of monomer unit such as one or more spacer units as described herein.

The leader may be configured or designed in order to promote the unbinding of the target polynucleotide from the polynucleotide binding site of the motor protein when the motor protein is in the vicinity of the leader sequence, e.g. when the motor protein contacts the leader.

In such embodiments, the motor protein typically has a lower affinity for the leader than for the target polynucleotide, i.e. than for the portion of the target polynucleotide to be characterised. In some embodiments the leader has a different structure to the target polynucleotide. In some embodiments the leader comprises a different type of nucleotide to the target polynucleotide.

In some embodiments the leader is charged. In some embodiments the leader is uncharged. In some embodiments the leader is negatively charged or positively charged, typically negatively charged.

In some embodiments the leader is polymeric. In some embodiments the leader is a linear polymeric group. In some embodiments the leader is a charged polymer, e.g. a negatively charged polymer. In some embodiments the leader comprises a polymer such as a polynucleotide, for instance DNA or RNA, a modified polynucleotide (such as abasic DNA), PNA, LNA, polyethylene glycol (PEG) or a polypeptide. In such embodiments the leader may be from 10 to 150 monomer units (e.g. ethylene glycol or saccharide units) in length, such as from 20 to 120, e.g. 30 to 100, for example 40 to 80 such as 50 to 70 monomer units (e.g. ethylene glycol or saccharide units) in length.

In some embodiments the leader may comprise a single-stranded polynucleotide region without significant secondary structure. For example, the leader sequence typically does not form a hairpin or G-quadruplex and thus is amenable to being captured by a nanopore.

In some embodiments the leader is or comprises a polynucleotide. In embodiments wherein the leader is a polynucleotide the leader may be the same sort of polynucleotide as the target polynucleotide, or it may be a different type of polynucleotide. For example, the target polynucleotide may be DNA and the leader may be RNA or vice versa. In some embodiments the target polynucleotide comprises or consists of DNA (e.g. dsDNA) and the leader does not consist of DNA, although in some embodiments the leader may comprise one or more nucleotides in addition to other monomer units; for example the leader may comprise one or more spacers as described herein). In some embodiments the target polynucleotide comprises or consists of ssDNA and the leader does not consist of ssDNA (e.g. the leader may comprise one or more spacers as described herein). In some embodiments the target polynucleotide comprises or consists of DNA and the leader also consists of DNA.

In some embodiments, the leader sequence comprises a single strand of DNA, such as a poly dT section. The leader sequence can be any length, but is typically 10 to 150 nucleotides in length, such as from 20 to 120, 30 to 100, 40 to 80 or 50 to 70 nucleotides in length.

For example, in some embodiments the target polynucleotide comprises deoxyribonucleotides (DNA). In such embodiments, the leader may comprises one or more nucleotides lacking both nucleobase and sugar moieties (e.g. a spacer moiety). Suitable spacer moieties are described in more detail herein, and include C2 spacers, C3 spacers, C6 spacers, iSp9 spacers, iSp18 spacers etc. For example, a leader may comprise about 5 to about 100, such as from about 10 to about 50, e.g. from about 20 to about 40 spacers as described herein, e.g. C3, iSp9 and/or iSp18. Alternatively or additionally, the leader may comprise ribonucleotides (RNA), peptide nucleotides (PNA), glycerol nucleotides (GNA), threose nucleotides (TNA), locked nucleotides (LNA), bridged nucleotides (BNA), or abasic nucleotides. In some embodiments the leader may comprise one or more nucleotides having a modified phosphate linkage (e.g. comprising a methylphosphonate or phosphothiorate linkage).

In some other embodiments the target polynucleotide comprises ribonucleotides (RNA). In such embodiments, the leader may comprises one or more spacers as defined above, deoxyribonucleotides (DNA), peptide nucleotides (PNA), glycerol nucleotides (GNA), threose nucleotides (TNA), locked nucleotides (LNA), bridged nucleotides (BNA), abasic nucleotides or nucleotides comprising a modified phosphate linkage.

Typically, the target polynucleotide comprises deoxyribonucleotides (DNA) and the leader comprises one or more spacer moieties (e.g. C3 spacers) and/or one or more ribonucleotides.

The leader may comprise just one type of polynucleotide that is different to the target polynucleotide. For example, when the target polynucleotide is DNA the leader may comprise spacer moieties or RNA. The leader may comprise more than one type of polynucleotide that is different to the target polynucleotide. For example, when the target polynucleotide is DNA the leader may comprise spacer moieties and RNA. The leader may comprise portions which are of the same type of polynucleotide as the target polynucleotide. For example, when the target polynucleotide is DNA, the leader may comprise in addition to spacer polynucleotides or RNA, portions of DNA. Such portions may be referred to as “traps”; i.e. a leader based on spacer (e.g. C3 spacer) and/or RNA (e.g. 2′-methoxy uridine) polynucleotides may comprise one or more DNA traps. A trap typically comprises from 1 to 10 nucleotides, such as from 1 to 6 nucleotides e.g. 1, 2, 3, 4 or 5 nucleotides such as from 1 to 3 nucleotides. When the target polynucleotide is DNA, the leader may therefore comprise one or more RNA (e.g. 2′-methoxyuridine) and/or spacer (e.g. C3 spacer) moieties and one or more DNA (e.g. thymidine) traps of from 1 to 10 nucleotides in length.

In some embodiments the leader comprises one or more stalling units as described herein. In some embodiments the leader may comprise one or more abasic spacers i.e. one or more spacers in which the bases are removed from one or more nucleotides in the polynucleotide adapter. In some embodiments the leader may comprise peptide nucleic acid (PNA), glycerol nucleic acid (GNA), threose nucleic acid (TNA), locked nucleic acid (LNA) or a synthetic polymer with nucleotide side chains. In some embodiments the leader may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A leader may comprise any combination of these groups. Many of these groups are commercially available from IDT® (Integrated DNA Technologies®). For example, C3, iSp9 and iSp18 spacers are all available from IDT®. A leader may comprise any number of the above groups. For example, a leader may comprise about 5 to about 100, such as from about 10 to about 50, e.g. from about 20 to about 40 spacers as described herein, e.g. C3, iSp9 and/or iSp18.

In some embodiments a leader comprises one or more spacers as described herein (e.g. from about 20 to about 40 spacers as described herein, e.g. C3, iSp9 and/or iSp18 and/or a region of homopolymeric polynucleotide e.g. a poly dT section of from about 20 to about 40 nucleotides in length); a heteropolymeric polynucleotide section; a stalling section for stalling a motor protein, wherein said stalling section may comprise one or more stalling units as described herein (e.g. from about 20 to about 40 spacers as described herein, e.g. C3, iSp9 and/or iSp18); and a polynucleotide motor protein binding site.

Those skilled in the art will also appreciate that when the leader comprises a polynucleotide strand, the sequence of the leader is typically not determinative and can be controlled or chosen according to the motor protein and other experimental conditions such as any polynucleotide to be characterised. Exemplary sequences are provided solely by way of illustration in the examples, such as in example 3. For example, the leader may comprise a sequence such as one or more of SEQ ID NOs: 53, 54 or 55, 56/67, 57 or 58/68, or a polynucleotide sequence having at least 20%, such as at least 30%, e.g. at least 40% such as at least 50%, e.g. at least 60% such as at least 70%, e.g. at least 80%, for example at least 90% e.g. at least 95% sequence similarity or identity to one or more of SEQ ID NOs: 53, 54 or 55, 56/67, 57 or 58/68. The sequence of the leader can typically be altered without negatively affecting the efficacy of the methods provided herein.

In some embodiments the leader is directly attached to the target polynucleotide. Usually the leader is attached to the target polynucleotide using an adapter; i.e. in some embodiments the leader is comprised in an adapter as described herein and the adapter is attached to the target polynucleotide. Suitable adapters are described in more detail herein. In some embodiments the leader is attached to the group of the adapter which attaches to the target polynucleotide by chemistry as described herein.

In some embodiments the leader is attached to the first end of the polynucleotide by a chemical bond. In some embodiments the leader is attached to the first end of the polynucleotide by a covalent bond. In some embodiments the leader is attached to the first end of the polynucleotide by a linker. Any suitable linker may be used. In some embodiments the linker is a polynucleotide as described herein. In some embodiments the linker is a synthetic polymer such as a PEG. In some embodiments the linker is a polynucleotide of the same type of polynucleotide as the target polynucleotide (e.g. in some embodiments the target polynucleotide conjugate comprises DNA and the linker comprises DNA) and the leader comprises one or more nucleotides of a different sort to those comprised in the polynucleotide portion of the conjugate. In some embodiments the target polynucleotide comprises DNA, the linker comprises DNA, and the leader comprises one or more non-DNA nucleotides as described herein, e.g. one or more spacers as described herein.

In some embodiments the leader is attached to the 5′ end of the target polynucleotide. In some embodiments the leader is attached to the 3′ end of the target polynucleotide.

In some embodiments the target polynucleotide has a naturally occurring reactive functional group which can be used to attach the leader.

The attachment chemistry between the leader and the polynucleotide in the conjugate is not particularly limited. Any suitable combination of reactive functional groups can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxymethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like.

Other suitable chemistry for attaching the leader to the polynucleotide includes click chemistry. Many suitable click chemistry reagents are known in the art. Suitable examples of click chemistry include, but are not limited to, the following:
(a) copper (I)-catalyzed azide-alkyne cycloadditions (azide alkyne Huisgen cycloadditions);
(b) strain-promoted azide-alkyne cycloadditions; including alkene and azide [3+2] cycloadditions; alkene and tetrazine inverse-demand Diels-Alder reactions; and alkene and tetrazole photoclick reactions;
(c) copper-free variant of the 1,3 dipolar cycloaddition reaction, where an azide reacts with an alkyne under strain, for example in a cyclooctane ring such as in bicycle [6.1.0] nonyne (BCN);
(d) the reaction of an oxygen nucleophile on one linker with an epoxide or aziridine reactive moiety on the other; and
(e) the Staudinger ligation, where the alkyne moiety can be replaced by an aryl phosphine, resulting in a specific reaction with the azide to give an amide bond.

Any reactive group may be used to attach the leader to the polypeptide. Some suitable reactive groups include [1, 4-Bis[3-(2-pyridyldithio)propionamido]butane; 1,11-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis[2-(4-azidosalicylamido)ethyl]disulphide; 3-(2-pyridyldithio) propionic acid N-hydroxysuccinimide ester; 4-maleimidobutyric acid N-hydroxysuccinimide ester; Iodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

In some embodiments the reactive functional group is comprised in the leader and the target functional group is comprised in the first end of the polynucleotide prior to the attachment step. In other embodiments the reactive functional group is comprised in the first end of the polynucleotide and the target functional group is comprised in the leader prior to the conjugation step. In some embodiments the reactive functional group is attached directly to the polynucleotide. In some embodiments the reactive functional group is attached to the polynucleotide via a spacer. Any suitable spacer can be used. Suitable spacers include for example alkyl diamines such as ethyl diamine, etc.

### Stalling Motor Proteins

As explained above, in some embodiments the methods provided herein may comprise characterisation of a target polynucleotide having a motor protein stalled thereon at a stalling moiety. In some embodiments the leader comprises a stalling moiety and the motor protein is bound to the leader by being stalled on the leader at the stalling moiety.

Any suitable stalling moiety can be used in the methods provided herein. In some embodiments the stalling moiety comprises a stalling site as described herein. In some embodiments the stalling site comprises one or more stalling units.

Any suitable stalling units can be used. A stalling unit typically provides an energy barrier which impedes movement of the motor protein. For example, a stalling unit may stall a motor protein by reducing the traction of the motor protein on the polynucleotide. This may be achieved for instance by using an abasic “spacer” i.e. a stalling unit in which the bases are removed from one or more nucleotides. A stalling unit may physically block movement of a motor protein, for instance by introducing a bulky chemical group to physically impede the movement of the protein.

In some embodiments, a stalling unit may comprise a linear molecule, such as a polymer. Typically, such a stalling unit has a different structure from the target polynucleotide. For instance, if the target polynucleotide is DNA, the or each stalling unit typically does not comprise DNA. In particular, if the target polynucleotide is deoxyribonucleic acid (DNA) or ribonucleic acid (RNA), the or each stalling unit preferably comprises peptide nucleic acid (PNA), glycerol nucleic acid (GNA), threose nucleic acid (TNA), locked nucleic acid (LNA), bridged nucleic acid (BNA) or a synthetic polymer with nucleotide side chains. In some embodiments, a stalling unit may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A stalling site may comprise any combination of these groups. Many of these groups are commercially available from IDT® (Integrated DNA Technologies®). For example, C3, iSp9 and iSp18 spacers are all available from IDT®. A stalling site may comprise any number of the above groups as stalling units. For instance, a stalling site may comprise from 1 to about 12 or more (e.g. from about 1 to about 8, for instance from 1 to about 6 such as from 1 to about 4) of such stalling units.

In some embodiments, a stalling unit may comprise one or more chemical groups which cause the a motor protein to stall. In some embodiments, suitable chemical groups are one or more pendant chemical groups. The one or more chemical groups may be attached to one or more nucleobases in the polynucleotide. The one or more chemical groups may be attached to the backbone of the polynucleotide. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

In some embodiments, a stalling unit may comprise a polymer. In some embodiments the stalling unit may comprise a polymer which is a polypeptide or a polyethylene glycol (PEG).

In some embodiments, a stalling unit may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic residues can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more stalling units do not comprise any abasic nucleotides.

Suitable stalling units can be designed or selected depending on the nature of the polynucleotide/polynucleotide adapter, the motor protein and the conditions under which the method is to be carried out. For example, many polynucleotide processing proteins process DNA in vivo and such proteins may typically be stalled using anything that is not DNA.

In some embodiments of the provided methods the motor protein is thus stalled at a stalling site comprising one or more stalling units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
spacer units selected from nitroindoles, inosines, acridines, 2-aminopurines, 2-6-diaminopurines, 5-bromo-deoxyuridines, inverted thymidines (inverted dTs), inverted dideoxy-thymidines (ddTs), dideoxy-cytidines (ddCs), 5-methylcytidines, 5-hydroxymethylcytidines, 2′-O-Methyl RNA bases, Iso-deoxycytidines (Iso-dCs), Iso-deoxyguanosines (Iso-dGs), C3 (OC₃H₆OPO₃) groups, photo-cleavable (PC) [OC₃H₆—C(O)NHCH₂—C₆H₃NO₂—CH(CH₃)OPO₃] groups, hexandiol groups, spacer 9 (iSp9) [(OCH₂CH₂)₃OPO₃] groups, spacer 18 (iSp18) [(OCH₂CH₂)₆OPO₃] groups; and thiol connections; and
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups.

Stalling moieties as described herein can also be used to configure a leader to be suitable for use in the disclosed re-reading methods. As explained above, in some embodiments of such methods a leader sequence as described herein is configured or designed in order to promote the unbinding of the target polynucleotide from the polynucleotide binding site of the motor protein when the motor protein is in the vicinity of the leader sequence, e.g. when the motor protein contacts the leader sequence. In some embodiments the leader sequence may comprise any of the spacer moieties described above.

### Destalling Motor Proteins

In some embodiments, the methods provided herein comprise contacting a stalling moiety as described herein with the detector (e.g. with a nanopore) thereby destalling a motor protein which may be stalled at a leader attached to the target polynucleotide. Once destalled, the motor protein can control the movement of the polynucleotide in the first direction with respect to the first and second openings of the detector (e.g. when the detector is or comprises a nanopore the motor protein can control the movement of the polynucleotide out of the nanopore) as described in more detail herein.

In its simplest form, contacting the stalling moiety with the detector e.g. a nanopore may destall the motor protein from the stalling moiety. However, in some embodiments the method comprises actively destalling the motor protein as described herein.

In some embodiments, destalling the motor protein comprises applying a destalling force to the polynucleotide, wherein the destalling force is lower in magnitude and/or of opposite direction to a read force, wherein the read force is the force applied whilst the motor protein controls the movement of the target polynucleotide and the measurements to determine one or more characteristics of the polynucleotide are taken.

For example, the read force may typically be provided as a voltage potential of from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. The destalling force is typically lower in magnitude than the read force. For example, the destalling force may be from about −100 mV to +100 mV, such as from about −50 mV to about +50 mV, e.g. from about −25 mV to about +25 mV.

For example, in some embodiments the read force is a voltage potential of from +50 mV to +300 mV, more preferably in the range of +100 mV to +200 mV such as from +120 mV to +150 mV, and the destalling force is a voltage potential of from −50 to +50 mV such as from −40 mV to +40 mV, e.g. from −20 mV to +20 mV such as 0 mV.

In some embodiments the destalling force is opposite in direction to the read force. For example in some embodiments the read force is applied as a positive voltage potential and the destalling force is applied as a negative voltage potential. In other embodiments the read force is applied as a negative voltage potential and the destalling force is applied as a positive potential. When the destalling force is of opposite direction to the read force it may be of equal magnitude to the read force or may be of lower magnitude to the read force.

In some embodiments the destalling force is applied at zero potential. For example in some embodiments the read force is applied as a positive voltage potential and the destalling force is applied at zero applied potential. In other embodiments the read force is applied as a negative voltage potential and the destalling force is applied at zero applied potential.

In some embodiments the destalling force is applied for a time sufficient for the motor protein to destall from the stalling moiety. In some embodiments the destalling force is applied for between 1 ms to about 10 s, such as from about 10 ms to about 1 s, e.g. from about 100 ms to about 700 ms such as from about 300 ms to about 500 ms.

In some embodiments destalling the motor protein comprises altering the applied force one or more times between the destalling force and the read force. In some embodiments altering the applied force in this manner comprises stepping or ramping the applied potential between the destalling force and the read force. When ramped, any suitable waveform can be used, e.g. the ramp may be a linear ramp, an exponential ramp or a sigmoidal ramp.

In some embodiments the applied force is stepped between a single destalling force and the read force. In some embodiments the applied force is stepped between a series of different destalling forces and the read force. In some embodiments the applied force is stepped between a series of increasing destalling forces and the read force. The destalling forces at each step may be any suitable destalling force, e.g. any of the destalling forces described herein; and at each step may be applied for any suitable time duration e.g. any time duration as described herein.

In some embodiments the destalling force is the same as the read force. This is also referred to as destalling in a “free running” setup.

In some embodiments the motor protein is stalled at a stalling site comprising one or more stalling units and one or more pausing moieties; and wherein contacting the one or more pausing moieties with the detector e.g. with a nanopore retards the movement of the polynucleotide with respect to the detector thereby causing the motor protein to destall from the one or more stalling units. Such embodiments are suitable for use in a free running setup.

In some embodiments the pausing moiety provides an energy barrier which impedes movement of the polynucleotide with respect to the detector e.g. through a nanopore. For example, a pausing moiety may impede movement of the polynucleotide through a nanopore by providing a physical block that needs to be removed before the polynucleotide can pass through the nanopore.

Without being bound by theory, the inventors believe that the pausing moiety retards the movement of the polynucleotide with respect to the first and second openings of the detector (e.g. when the detector is or comprises a nanopore, through the nanopore) for sufficient time for the motor protein to overcome the stalling unit(s) and destall.

In some embodiments the pausing moiety comprises one or more pausing units comprising a polynucleotide secondary structure, preferably a hairpin or G-quadruplex (TBA). Such secondary structures prevent the free passage of the polynucleotide through the nanopore. Contacting the pausing moiety with the nanopore causes the secondary structure to dissociate (e.g. to unwind). The time taken for the secondary structure to dissociate allows the motor protein to destall from the stalling unit(s).

In some embodiments the pausing moiety comprises one or more pausing units comprising a hybridised oligonucleotide. The oligonucleotide may hybridise to the target polynucleotide and prevent movement of the target polynucleotide through the nanopore. Contacting the pausing moiety with the nanopore causes the dissociation of the hybridised oligonucleotide from the target polynucleotide. The time taken for the hybridised oligonucleotide to dissociate from the target polynucleotide allows the motor protein to destall from the stalling unit(s).

In some embodiments the pausing moiety comprises one or more pausing units comprising a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides. The nucleic acid analog may be provided in line with the target polynucleotide or may be hybridised or otherwise attached to the target polynucleotide. When the nucleic acid analog is provided in line with the target polynucleotide, contacting the pausing moiety with the nanopore causes the nucleic acid analog to pass through the nanopore. The time taken for the nucleic acid analog to move with respect to the detector e.g. when the detector is or comprises a nanopore, the time to pass through the pore allows the motor protein to destall from the stalling unit(s). When the nucleic acid analog is hybridised to the target polynucleotide, contacting the pausing moiety with the detector (e.g. the nanopore) typically causes the nucleic acid analog to dissociate from the target polynucleotide such that the target polynucleotide can move with respect to the detector (e.g. by passing through the nanopore). The time taken for the nucleic acid analog to dissociate from the polynucleotide allows the motor protein to destall from the stalling unit(s).

In some embodiments the pausing moiety comprises one or more pausing units comprising a chemical group such as fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups. The chemical groups may be attached to the target polynucleotide and prevent the movement of the target polynucleotide with respect to the detector (e.g. through a nanopore). In some embodiments contacting the pausing moiety with the detector (e.g. a nanopore) causes the chemical group to be removed from the target polynucleotide. In some embodiments contacting the pausing moiety with the detector (e.g. a nanopore) causes the chemical group to move through the first and second openings of the detector, e.g. to be passed through the nanopore. The time taken for the chemical group to be removed from the target polynucleotide and/or to pass through the first and second openings (e.g. of the nanopore) allows the motor protein to destall from the stalling unit(s).

In some embodiments the pausing moiety comprises one or more pausing units comprising a polynucleotide binding protein. Suitable polynucleotide binding proteins are described in more detail herein. The polynucleotide binding protein may be bound to the polynucleotide and prevent movement of the polynucleotide with respect to the detector (e.g. through a nanopore). Contacting the pausing moiety with the detector (e.g. a nanopore) retards the movement of the polynucleotide with respect to the detector (e.g. through a nanopore), e.g. as the polynucleotide binding protein moves to contact the motor protein. The time taken to do allows the motor protein to destall from the stalling unit(s). Without being bound by theory the inventors also believe that the pausing moiety often determines the conformation of the polynucleotide at the stalling unit(s). This is particularly the case when the stalling moiety comprises a linear group such as one or more spacer 18 (iSp18) [(OCH₂CH₂)₆OPO₃]. Without being bound by theory, it is believed that when such stalling unit(s) are in contact with a detector such as a nanopore any applied force across the pore (e.g. an applied voltage field) can cause the stalling moiety to stretch out in an approximately linear manner. In this conformation, the motor protein is typically incapable of passing over the stalling moiety to destall. However, when the target polynucleotide is paused at a pausing moiety the environment at the stalling unit is believed to be similar to that in solution and the stalling unit may adopt a more compact pseudo-random coil configuration. In this configuration it may be easier for the motor protein to overcome the stalling unit and destall.

Accordingly, in some embodiments the motor protein is stalled at a stalling site comprising one or more stalling units and one or more pausing units independently selected from:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups; and
a polynucleotide binding protein;

and contacting the one or more pausing moieties with a detector such as a nanopore retards the movement of the polynucleotide with respect to the detector (e.g. through the nanopore) thereby causing the motor protein to destall from the one or more stalling units.

### Motor Protein

As those skilled in the art will appreciate, any suitable motor protein can be used in the methods and products provided herein.

The motor protein may be any protein that is capable of binding to a polynucleotide and controlling its movement with respect to a detector, e.g. a nanopore, e.g. through the pore.

In more detail, motor proteins such as helicases can typically control the movement of DNA in at least two active modes of operation (when is provided with all the necessary components to facilitate movement e.g. ATP and Mg²⁺) and one inactive mode of operation (when not provided with the necessary components to facilitate movement; or when the motor protein is modified in order to prevent the active mode).

When provided with all the necessary components to facilitate movement, a motor protein may move along a polynucleotide such as DNA in either a 5′-3′ direction or a 3′-5′ direction. Many motor proteins process polynucleotides such as DNA in a 5′-3′ direction. Motor proteins which control the movement of polynucleotides in this manner are typically suitable for use in the methods provided herein.

However, when a motor protein is not provided with the necessary components to facilitate movement, or is modified in order to prevent it from actively controlling the movement of the polynucleotide with respect to the nanopore, it can still passively control the movement of the polynucleotide with respect to the nanopore. For example, the motor protein can bind to the polynucleotide and act as a brake slowing the movement of the polynucleotide when it is pulled into the pore by an applied field (e.g. by the first force in the methods provided herein). In the “inactive” mode it typically does not matter whether the polynucleotide is captured either 3′ or 5′ down (i.e. moves through the nanopore in a 5′-3′ direction or in a 3′-5′ direction), as the applied force provides the impetus to move the polynucleotide through the nanopore. However, in such embodiments the motor protein may still control the movement of the polynucleotide with respect to the nanopore e.g. by acting as a brake. When in the inactive mode the movement control of a polynucleotide by a motor protein can be described in a number of ways including ratcheting, sliding and braking. Typically the methods provided herein do not comprise the use of a motor protein operating in the passive mode. However in embodiments of the methods provided herein which use a polynucleotide binding protein the polynucleotide binding protein may be a motor protein operating in the passive mode.

As explained above, some embodiments of the methods provided herein also comprise the use of a polynucleotide binding protein as a pausing moiety to impede the movement of the polynucleotide strand through the nanopore. In some embodiments a polynucleotide binding protein may be a motor protein as described herein. In other embodiments a polynucleotide binding protein may be a protein which binds to polynucleotides but which does not have polynucleotide processing capacity; i.e. in some embodiments it is not a motor protein.

A polynucleotide-handling enzyme is a polypeptide that is capable of interacting with a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. A motor protein as used herein may be, or may be derived from a polynucleotide handling enzyme. A polynucleotide binding protein may be, or may be derived from a polynucleotide-handling enzyme.

In one embodiment, the motor protein and/or polynucleotide binding protein are independently derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

Typically, the motor protein and/or polynucleotide binding protein are each independently a helicase, a polymerase, an exonuclease, a topoisomerase, or a variant thereof.

In some embodiments, the motor protein and/or polynucleotide binding protein may be modified to prevent the motor protein disengaging from the polynucleotide. Thus, in some embodiments of such methods, the target polynucleotide does not disengage from the motor protein.

As used herein, the term “disengaging” refers to the dissociation of the motor protein from the target polynucleotide. Thus, a motor protein may be modified to prevent it from dissociating from the target polynucleotide, e.g. into the reaction medium. It is important to distinguish potential “disengagement” of a motor protein from “unbinding” of a motor protein from a target polynucleotide. As used herein, “unbinding” refers to the transient release of the target polynucleotide the active site of the motor protein (described in more detail herein) but does not imply disengagement. Thus, for example, a motor protein may be modified to prevent the motor protein from disengaging from a polynucleotide, but without preventing the motor protein from unbinding from the polynucleotide. When unbound, the motor protein remains engaged with the target polynucleotide. For example, the motor protein may remain engaged with the target polynucleotide (i.e. it may be prevented from disengaging from the target polynucleotide).

because it is topologically closed around the target polynucleotide. The polynucleotide binding site may remain free to bind or unbind the target polynucleotide such that the motor protein may bind or unbind to the target polynucleotide, whilst the motor protein remains engaged with the target polynucleotide. When the motor protein is unbound from the target polynucleotide it may be able to move on (e.g., along) the target polynucleotide under an applied force and may be capable of re-binding to the target polynucleotide. When engaged on the target polynucleotide but unbound from the target polynucleotide, the motor protein is not capable of dissociating from the target polynucleotide.

The motor protein and/or polynucleotide binding protein can be adapted to prevent disengagement in any suitable way. For example, the motor protein and/or polynucleotide binding protein can be loaded on the polynucleotide and then modified in order to prevent it from disengaging from the polynucleotide. Alternatively, the motor protein and/or polynucleotide binding protein can be modified to prevent it from disengaging from the polynucleotide before it is loaded onto the polynucleotide. Modification of a motor protein and/or a polynucleotide binding protein in order to prevent it from disengaging from a polynucleotide can be achieved using methods known in the art, such as those discussed in WO 2014/013260 and WO 2015/110813, each of which is hereby incorporated by reference in its entirety, and with particular reference to passages describing the modification of motor proteins such as helicases in order to prevent them from disengaging with polynucleotide strands. For example, a motor protein and/or polynucleotide binding protein can be modified by treating with tetramethylazodicarboxamide (TMAD). Various other closing moieties are described in more detail herein.

For example, a motor protein and/or a polynucleotide binding protein may have a polynucleotide-unbinding opening; e.g. a cavity, cleft or void through which a polynucleotide strand may pass when the motor protein/polynucleotide binding protein disengages from the strand. In some embodiments, the polynucleotide-unbinding opening is the opening through which a polynucleotide may pass when the motor protein/polynucleotide binding protein disengages from the polynucleotide. In some embodiments, the polynucleotide-unbinding opening for a given motor protein/polynucleotide binding protein can be determined by reference to its structure, e.g. by reference to its X-ray crystal structure. The X-ray crystal structure may be obtained in the presence and/or the absence of a polynucleotide substrate. In some embodiments, the location of a polynucleotide-unbinding opening in a given motor protein/polynucleotide binding protein may be deduced or confirmed by molecular modelling using standard packages known in the art. In some embodiments, the polynucleotide-unbinding opening may be transiently produced by movement of one or more parts e.g. one or more domains of the motor protein.

The motor protein/polynucleotide binding protein may be modified by closing the polynucleotide-unbinding opening. The polynucleotide-unbinding opening may be closed with a closing moiety. Closing the polynucleotide-unbinding opening may therefore prevent the motor protein/polynucleotide binding protein from disengaging from the polynucleotide. For example, the motor protein and/or polynucleotide binding protein may be modified by covalently closing the polynucleotide-unbinding opening. However, as explained above closing the polynucleotide-unbinding opening does not necessarily prevent the target polynucleotide from unbinding from the polynucleotide binding site of the motor protein. In some embodiments, a preferred protein for addressing in this way is a helicase.

In some embodiments, the motor protein may be modified to prevent the target polynucleotide disengaging from the target polynucleotide. The motor protein may be modified in any suitable manner.

Without being bound by theory, the inventors believe that promoting unbinding and retarding re-binding may promote re-reading. Without being bound by theory, the inventors believe that this may be because each step that a motor protein takes on a target polynucleotide is associated with a probability of the motor protein unbinding from the polynucleotide. The likelihood of such unbinding may be identified with the so-called off-rate. Increasing the off-rate is believed to promote drop-back of the motor protein with respect to the polynucleotide strand. Similarly, and again without being bound by theory, the inventors believe that once unbound from a target polynucleotide, the distance that the motor protein may move along the target polynucleotide before re-binding is associated with the on-rate. Thus, re-reading may be promoted by increasing the off-rate and decreasing the on-rate of the motor protein with respect to the target polynucleotide. Tailoring the off- and on-rates of the motor protein for a given type of polynucleotide is within the abilities of those of skill in the art in view of the disclosure herein. Accordingly, the motor protein may be modified to promote unbinding of the target polynucleotide from the polynucleotide binding site of the motor protein and/or to retard re-binding of the target polynucleotide to the polynucleotide binding site of the motor protein. In some embodiments the motor protein is modified to both promote unbinding of the target polynucleotide from the polynucleotide binding site of the motor protein and to retard re-binding of the target polynucleotide to the polynucleotide binding site of the motor protein.

In some embodiments the motor protein may be modified with a closing moiety for (i) topologically closing the polynucleotide binding site of the motor protein around the target polynucleotide and (ii) promoting unbinding of the target polynucleotide from the polynucleotide binding site of the motor protein and/or retarding re-binding of the target polynucleotide to the polynucleotide binding site of the motor protein. The motor protein may be modified in any suitable manner to facilitate attachment of such a closing moiety.

In some embodiments a closing moiety may comprise a bifunctional cross-linking moiety. The closing moiety may comprise a bifunctional cross-linker. The bifunctional crosslinker may attach at two points on the motor protein and close the polynucleotide-unbinding opening of the motor protein thereby preventing disengagement of the polynucleotide from the motor protein whilst allowing unbinding of the polynucleotide from the polynucleotide-binding site of the motor protein.

The closing moiety may attach at any suitable positions on the motor protein. For example, the closing moiety may crosslink two amino acid residues of the motor protein. Typically, at least one amino acid crosslinked by the closing moiety is a cysteine or a non-natural amino acid. The cysteine or non-natural amino acid may be introduced into the motor protein by substitution or modification of a naturally occurring amino acid residue of the motor protein. Methods for introducing non-natural amino acids are well known in the art and include for example native chemical ligation with synthetic polypeptide strands comprising such non-natural amino acids. Methods for introducing cysteines into a motor protein are likewise within the capability of one of skill in the art, for example using techniques disclosed in references such as Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

In some embodiments the closing moiety has a length of from about 1 Å to about 100 Å. The length of the closing moiety may be calculated according to static bond lengths or more preferably using molecular dynamics simulations. The length may for example be from about 2 Å to about 80 Å, such as from about 5 Å to about 50 Å, e.g. from about 8 to about 30 Å such as from about 10 to about 25 Å or about 20 Å, e.g. about 1, 2, 3, 4, 5, 6, 7, 8, 9, 10, 11, 12, 13, 14, 15, 16, 17, 18, or 19 Å.

Without in any way being bound by theory, the inventors consider that in general longer closing moieties may increase the off-rate of the motor protein from the polynucleotide and thus promote re-reading.

In some embodiments the closing moiety comprises a bond. In some embodiments the closing moiety comprises a disulphide bond. A disulphide bond may be formed by treating the motor protein with any suitable reagent such as TMAD.

In some embodiments the closing moiety comprises a reagent which forms bonds between two click chemistry groups on the motor protein. Examples of click chemistry reagents are provided herein.

In some embodiments the closing moiety comprises a protein. For example, biotin groups may be present on the motor protein and the closing moiety may comprise streptavidin. Tags such as snoop-tag or spy-tag may be present on the motor protein and the closing moiety may comprise a protein such as snoop-catcher or spy-catcher, respectively.

In some embodiments the closing moiety comprises a structure of formula [A-B-C], wherein A and C are each independently reactive functional groups for reacting with amino acid residues in the motor protein and B is a linking moiety. In some embodiments the closing moiety comprises a link between thio groups e.g. thiol groups on cysteine residues. In some embodiments therefore A and C are cysteine-reactive functional groups. In some embodiments linking moiety B comprises a linear or branched, unsubstituted or substituted alkylene, alkenylene, alkynylene, arylene, heteroarylene, carbocyclylene or heterocyclylene moiety, which moiety is optionally interrupted by and/or terminated in one or more atoms or groups selected from O, N(R), S, C(O), C(O)NR, C(O)O, unsubstituted or substituted arylene, arylene-alkylene, heteroarylene, heteroarylene-alkylene, carbocyclylene, carbocyclylene-alkylene, heterocyclylene and heterocyclylene-alkylene; wherein R is selected from H, unsubstituted or substituted alkyl, and unsubstituted or substituted aryl. Typically R is H or methyl, more typically H.

Typically, an alkylene group is a C₁₋₂₀ alkylene group. Typically, an alkenylene group is a C₂₋₂₀ alkenylene group. Typically, an alkynylene group is a C₂₋₂₀ alkynylene group. Typically, an arylene group is a C₆₋₁₂ arylene group. Typically, a heteroarylene group is a 5- to 12-membered heteroarylene group. Typically, a carbocyclylene group is a C₅₋₁₂ carbocyclylene group. Typically, a heterocyclylene group is a 5- to 12-membered heterocyclylene group.

Typically, an alkylene, alkenylene, or alkynylene moiety may be uninterrupted or interrupted by or terminate in one or more atoms or groups selected from O, N(R), S, C(O), C(O)NR, and C(O)O and unsubstituted or substituted arylene. Usually, an alkylene, alkenylene, or alkynylene moiety may be uninterrupted or interrupted by or terminate in one or more atoms or groups selected from O and N(R) and unsubstituted or substituted arylene. More often, an alkylene, alkenylene, or alkynylene moiety may be uninterrupted or interrupted by or terminate in one or more O atoms.

For example, a linking moiety is often an unsubstituted or substituted C₁₋₁₀ alkylene, C₂₋₁₀ alkenylene, or C₂₋₁₀ alkynylene moiety which is uninterrupted or interrupted by or terminates in one or more O atoms.

In some embodiments, linking moiety B comprises an alkylene, oxyalkylene or polyoxyalkylene group and/or wherein A and C are each maleimide groups. The alkylene, oxyalkylene or polyoxyalkylene group may for example have a length of from about 5 Å to about 50 Å, e.g. from about 8 to about 30 Å such as from about 10 to about 25 Å.

For example, a linking moiety may comprise a PEG moiety such as (CH₂CH₂O)ₓ wherein x is from 1 to 10, e.g. from 1 to 5, e.g. 1, 2 or 3. Exemplary linking moieties are described in example 2 and include for example BMOE (1,2-bismaleimidoethane), BMOP (1,3-bismaleimidopropane), BMB (1,4-bismaleimidobutane), BM(PEG)2 (1,8-bismaleimido-diethyleneglycol) and BM (PEG) 3 (1,11-bismaleimido-triethyleneglycol).

Motor proteins suitable for being closed using a closing moiety as described above are discussed in more detail herein. In some preferred embodiments the motor protein is a helicase, e.g. a Dda helicase as described herein.

In one embodiment, the motor protein and/or the polynucleotide binding protein is or is derived from an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the motor protein and/or the polynucleotide binding protein is a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5) or a variant thereof. Modified versions of Phi29 polymerase that may be used in the invention are disclosed in U.S. Pat. No. 5,576,204.

In one embodiment the motor protein and/or the polynucleotide binding protein is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment, the motor protein and/or the polynucleotide binding protein is a helicase. Any suitable helicase can be used in accordance with the methods provided herein. For example, the or each enzyme used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtfK and hexameric enzyme complexes, or multisubunit complexes such as RecBCD. In one embodiment the motor protein is a Dda (DNA-dependent ATPase) helicase.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment a helicase may comprise the sequence shown in SEQ ID NO: 6 (Trwc Cba) or a variant thereof, the sequence shown in SEQ ID NO: 7 (Hel308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (AM1) G1G2 (i.e. deletion of M1 and then addition of G1 and G2).

Typically, a motor protein or polynucleotide binding protein may have a fuel binding site. The active unwinding of DNA may be coupled to fuel hydrolysis, e.g. in the motor protein.

Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for the motor protein is a factor that allows the motor protein to function. The cofactor is preferably a divalent metal cation. The divalent metal cation is preferably Mg²⁺, Mn²⁺, Ca²⁺ or Co²⁺. The cofactor is most preferably Mg²⁺.

In some embodiments the polynucleotide binding protein is other than a motor protein as used herein. As used herein, the term polynucleotide binding protein and polynucleotide binding moiety can be used interchangeable.

For example, the polynucleotide binding protein or polynucleotide binding moiety may comprise one or more domains independently selected from helix-hairpin-helix (HhH) domains, eukaryotic single-stranded binding proteins (SSBs), bacterial SSBs, archaeal SSBs, viral SSBs, double-stranded binding proteins, sliding clamps, processivity factors, DNA binding loops, replication initiation proteins, telomere binding proteins, repressors, zinc fingers and proliferating cell nuclear antigens (PCNAs).

Helix-hairpin-helix (HhH) domains are polypeptide motifs that bind DNA in a sequence non-specific manner. Suitable domains include domain H (residues 696-751) and domain HI (residues 696-802) from Topoisomerase V from Methanopyrus kandleri (SEQ ID NO: 37). The polynucleotide binding moiety may be domains H-L of SEQ ID NO: 37 as shown in SEQ ID NO: 38 or a polynucleotide-binding variant thereof. The HhH domain may comprise the sequence shown in SEQ ID NO: 23 or 31 or 32 or a polynucleotide-binding variant thereof.

SSBs bind single stranded DNA with high affinity in a sequence non-specific manner. SSBs fall into the following lineage: Class; All beta proteins, Fold; OB-fold, Superfamily: Nucleic acid-binding proteins, Family; Single strand DNA-binding domain, SSB. The SSB may be from a eukaryote, such as from humans, mice, rats, fungi, protozoa or plants, from a prokaryote, such as bacteria and archaea, or from a virus. Eukaryotic SSBs are also known as replication protein A (RPAs). In most cases, they are hetero-trimers formed of different size units. Some of the larger units (e.g. RPA70 of Saccharomyces cerevisiae) are stable and bind ssDNA in monomeric form. Bacterial SSBs bind DNA as stable homo-tetramers (e.g. E. coli, Mycobacterium smegmatis and Helicobacter pylori) or homo-dimers (e.g. Deinococcus radiodurans and Thermotoga maritima). A few, such as the SSB encoded by the crenarchaeote Sulfolobus solfataricus, are homo-tetramers. Some SSBs from other species have been shown to be monomeric (Methanococcus jannaschii and Methanothermobacter thermoautotrophicum). Still other species of Archaea, including Archaeoglobus fulgidus and Methanococcoides burtonii, contain two open reading frames with sequence similarity to RPAs. Viral SSBs bind DNA as monomers.

The SSB is typically chosen or modified to have a carboxy-terminal (C-terminal) region which as no net negative charge or has a reduced net negative charge relative to the wild-type protein. Such SSBs typically do not block transmembrane pores. The C-terminal region of the SSB is typically about the last third, quarter, fifth or eighth of the SSB at the C-terminal end. The C-terminal region is typically from about the last 10 to about the last 60 amino acids of the C-terminal end of the SSB, e.g. from about the last 20 to last 40 such as last 30 amino acids of the C-terminal end of the SSB.

Examples of SSBs comprising a C-terminal region which does not have a net negative charge include the human mitochondrial SSB (HsmtSSB; SEQ ID NO: 33, the human replication protein A 70 kDa subunit, the human replication protein A 14 kDa subunit, the telomere end binding protein alpha subunit from Oxytricha nova, the core domain of telomere end binding protein beta subunit from Oxytricha nova, the protection of telomeres protein 1 (Pot1) from Schizosaccharomyces pombe, the human Pot1, the OB-fold domains of BRCA2 from mouse or rat, the p5 protein from phi29 (SEQ ID NO: 34); and polynucleotide-binding variants thereof. Examples of SSBs which can be modified in their C-terminal region to decrease the net negative charge include the SSB of E. coli (EcoSSB; SEQ ID NO: 35, the SSB of Mycobacterium tuberculosis, the SSB of Deinococcus radiodurans, the SSB of Thermus thermophiles, the SSB from Sulfolobus solfataricus, the human replication protein A 32 kDa subunit (RPA32) fragment, the CDC13 SSB from Saccharomyces cerevisiae, the Primosomal replication protein N (PriB) from E. coli, the PriB from Arabidopsis thaliana, the hypothetical protein At4g28440, the SSB from T4 (gp32; SEQ ID NO: 36), the SSB from RB69 (gp32; SEQ ID NO: 24), the SSB from T7 (gp2.5; SEQ ID NO: 25), and polynucleotide-binding variants thereof. Suitable modifications for decreasing the net negative charge are disclosed in WO 2014/013259.

Double-stranded binding proteins bind double stranded DNA with high affinity. Suitable double-stranded binding proteins include, but are not limited to Mutator S (MutS; NCBI Reference Sequence: NP_417213.1; SEQ ID NO: 39), Sso7d (Sufolobus solfataricus P2; NCBI Reference Sequence: NP_343889.1; SEQ ID NO: 40; Nucleic Acids Research, 2004, Vol 32, No. 3, 1197-1207), Sso10b1 (NCBI Reference Sequence: NP_342446.1; SEQ ID NO: 41), Sso10b2 (NCBI Reference Sequence: NP_342448.1; SEQ ID NO: 42), Tryptophan repressor (Trp repressor; NCBI Reference Sequence: NP_291006.1; SEQ ID NO: 43), Lambda repressor (NCBI Reference Sequence: NP_040628.1; SEQ ID NO: 44), Cren7 (NCBI Reference Sequence: NP_342459.1; SEQ ID NO: 45), major histone classes H1/H5, H2A, H2B, H3 and H4 (NCBI Reference Sequence: NP_066403.2, SEQ ID NO: 46), dsbA (NCBI Reference Sequence: NP_049858.1; SEQ ID NO: 47), Rad51 (NCBI Reference Sequence: NP_002866.2; SEQ ID NO: 48), sliding clamps and Topoisomerase V Mka (SEQ ID NO: 37) or a polynucleotide-binding variant of any of these proteins.

Other polynucleotide binding proteins include sliding clamps. Sliding clamps are typically multimeric proteins (homo-dimers or homo-trimers) that encircle dsDNA. Sliding clamps typically require accessory proteins (clamp loaders) to assemble them around the DNA helix in an ATP-dependent process. They also do not contact DNA directly, acting as a topological tether. Related to DNA sliding clamps are processivity factors, which are viral proteins that anchor their cognate polymerases to DNA, leading to a dramatic increase in the length of the fragments generated. They can be monomeric (as is the case for UL42 from Herpes simplex virus 1) or multimeric (UL44 from Cytomegalovirus is a dimer). UL42 typically comprises the sequence shown in SEQ ID NO: 26 or SEQ ID NO: 30 or a polynucleotide-binding variant thereof.

Another polynucleotide binding protein is the thioredoxin binding domain (TBD) of bacteriophage T7 DNA polymerase (residues 258-333). Binding of TBD to thioredoxin (e.g. from E. coli) causes the polypeptide to change conformation to one that binds DNA. Other polynucleotide binding proteins include the accessory proteins cisA from phage Φx174 and geneII protein from phage M13. These proteins have intrinsic DNA binding capabilities, some of them recognizing a specific DNA sequence. Other polynucleotide binding proteins include telomeric binding proteins.

Small DNA binding motifs (such as helix-turn-helix) recognize specific DNA sequences. In the case of the bacteriophage 434 repressor, a 62 residue fragment was engineered and shown to retain DNA binding abilities and specificity. Zinc fingers consist of around 30 amino-acids that bind DNA in a specific manner. Typically each zinc finger recognizes only three DNA bases, but multiple fingers can be linked to obtain recognition of a longer sequence.

Proliferating cell nuclear antigens (PCNAs) form a very tight clamp which slides up and down the dsDNA or ssDNA. The PCNA from crenarchaeota is a hetero-trimer of SEQ ID NOs: 27, 28 and 29. A polynucleotide binding protein may thus be a trimer comprising the sequences shown in SEQ ID NOs: 27, 28 and 29 or polynucleotide-binding variants thereof. Another PCNA sliding clamp (NCBI Reference Sequence: ZP_06863050.1; SEQ ID NO: 49) forms a dimer. The polynucleotide binding protein may thus be a dimer comprising SEQ ID NO: 49 or a polynucleotide-binding variant thereof.

The polynucleotide binding motif may be selected from any of the following:

| Name | Class | Organism | Structure | Sequence | Functional form | MW (Da) |
|---|---|---|---|---|---|---|
| SSBEco | ssb | Escherichia coli | 1QVC, 1EYG | P0AGE0 | homo-tetramer | 18975 |
| SSBBhe | ssb | Bartonella henselae | 3LGJ, 3PGZ | Q6G302 | homo-tetramer | 16737 |
| SSBCbu | ssb | Coxiella burnetii | 3TQY | Q83EP4 | homo-tetramer | 17437 |
| SSBTma | ssb | Thermathoga | 1Z9F | Q9WZ73 | homo-dimer | 16298 |
| | | maritima | | | | |
| SSBHpy | ssb | Helicobacter pylori | 2VW9 | O25841 | homo-tetramer | 20143 |
| SSBDra | ssb | Deinococcus | 1SE8 | Q9RY51 | homo-dimer | 32722 |
| | | radiodurans | | | | |
| SSBTaq | ssb | Thermus aquaticus | 2FXQ | Q9KH06 | homo-dimer | 30026 |
| SSBMsm | ssb | Mycobacterium | 3A5U, 1X3E | Q9AFI5 | homo-tetramer | 17401 |
| | | smegmatis | | | | |
| SSBSso | ssb/RPA | Sulfolobus | 1O7I | Q97W73 | homo-tetramer | 16138 |
| | | solfataricus | | | | |
| SSBMHsmt | ssb | Homo sapiens | 3ULL | Q04837 | homo-tetramer | 17260 |
| SSBMle | ssb | Mycobacterium | 3AFP | P46390 | homo-tetramer | 17701 |
| | | leprae | | | | |
| gp32T4 | ssb | Bacteriohage T4 | 1GPC | P03695 | monomer | 33506 |
| gp32RB69 | ssb | Bacteriophage RB69 | 2A1K | Q7Y265 | monomer | 33118 |
| gp2.5T7 | ssb | Bacteriohage T7 | 1JE5 | P03696 | monomer | 25694 |
| UL42 | processivity | Herpes virus 1 | 1DML | P10226 | monomer | 51159 |
| | factor | | | | | |
| UL44 | processivity | Herpes virus 5 | 1YYP | P16790 | homo-dimer | 46233 |
| | factor | (cytomegalovirus) | | | | |
| pf8 | processivity | KSHV | 3I2M | Q77ZG5 | homo-dimer | 42378 |
| | factor | | | | | |
| RPAMja | RPA | Methanococcus | 3DM3 | Q58559 | monomer | 73842 |
| | | jannaschii | | | | |
| RPAMma | RPA | Methanococcus | 3E0E, 2K5V | Q6LYF9 | monomer | 71388 |
| | | maripaludis | | | | |
| RPAMth | RPA | Methanothermobacter | | | monomer | 120000 |
| | | thermoautotrophicus | | | | |
| RPA70Sce | RPA | Saccharomyces | 1YNX | P22336 | hetero-trimer | 70348 |
| | | cerevisiae | | | | |
| RPAMbu1 | RPA | Methanococcoides | | Q12V72 | | 41227 |
| | | burtonii | | | | |
| RPAMbu2 | RPA | Methanococcoides | | Q12W96 | | 47082 |
| | | burtonii | | | | |
| RPA70Hsa | RPA | Homo sapiens | 1JMC | P27694 | hetero-trimer | 68138 |
| RPA14Hsa | RPA | Homo sapiens | 3KDF | P35244 | hetero-trimer | 13569 |
| gp45T4 | sliding clamp | Bacteriophage T4 | 1CZD | P04525 | homo-trimer | 24858 |
| BetaEco | sliding clamp | E. coli | 3BEP | P0A988 | homo-dimer | 40587 |
| PCNASce | sliding clamp | Saccharomyces | 1PLQ, 3K4X | P15873 | homo-dimer | 28916 |
| | | cerevisiae | | | | |
| PCNATko | sliding clamp | Thermococcus | 3LX1 | Q5JF32 | homo-dimer | 28239 |
| | | kodakaraensis | | | | |
| PCNAHvo | sliding clamp | Haloferax volcanii | 3IFV | D0VWY8 | homo-dimer | 26672 |
| PCNAPfu | sliding clamp | Pyrococcus furiosus | 1GE8 | O73947 | homo-dimer | 28005 |
| PCNAMbu | sliding clamp | Methanococcoides | | Q12U18 | homo-dimer | 27121 |
| | | burtonii | | | | |
| BetaMtu | sliding clamp | Mycobacterium | 3P16 | Q50790 | homo-dimer | 42113 |
| | | tuberculosis | | | | |
| BetaTma | sliding clamp | Thermotoga maritima | 1VPK | Q9WYA0 | homo-dimer | 40948 |
| BetaSpy | sliding clamp | Streptococcus | 2AVT | Q9EVR1 | homo-dimer | 41867 |
| | | pyrogenes | | | | |
| gp45RB69 | sliding clamp | Bacteriophage RB69 | 1B77 | O80164 | homo-trimer | 25111 |
| p55Hsa | DNA binding | Homo sapiens | 2G4C, 3IKL, | Q9UHN | monomer | 54911 |
| | protein | (mitochondrial) | 3IKM | | | |
| p55Dme | DNA binding | Drosophylla | | Q9VJV8 | monomer | 41027 |
| | protein | melanogaster | | | | |
| p55Xla | DNA binding | Xenopus laevis | | Q9W6G7 | monomer | 52283 |
| | protein | | | | | |
| RepDSau | replication | Staphylococcus | | P08115 | homo-dimer | 37874 |
| | initiation protein | aureus | | | | |
| G2P | replication | Enterobacteria | | P69546 | monomer | 46168 |
| | initiation protein | phage 1 | | | | |
| MutLEco | mismatch repair | Escherichia coli | 1BKN, 1B62, | P23367 | homo-dimer | 67924 |
| | protein | | 1B63 | | | |
| KuMtu | DNA repair protein | Mycobacterium | | O05866 | homo-dimer | 30904 |
| | | tuberculosis | | | | |
| OnTEBP | telomere binding | Oxytricha nova- | 1OTC | P29549 | hetero-dimer | 56082 |
| | protein | Alpha | | | | |
| | | Oxytricha nova-Beta | | P16458 | | 41446 |
| EcrTEBP | telomere binding | Euplotes crassus | | Q06183 | monomer | 53360 |
| | protein | | | | | |
| TteTEBP | telomere binding | Tetrachymena | | Q23FB9 | hetero-dimer | 53073 |
| | protein | termophila Alpha | | | | |
| | | Tetrachymena | | Q23FH0 | | 54757 |
| | | termophila Beta | | | | |
| pot1Spo | telomere binding | Schizosaccharomyces | | O13988 | monomer | 64111 |
| | proteins | pombe | | | | |
| Cdc13pSce | telomere binding | Saccharomyces | | C7GSV7 | monomer | 104936 |
| | proteins | cerevisiae | | | | |
| C1 | repressor | Bacteriophage 434 | | P16117 | homo-dimer | 10426 |
| LexA | repressor | Escherichia coli | 1LEB | P0A7C2 | homo-dimer | 22358 |

### Polynucleotide

The methods of the invention involve characterising a target polynucleotide as it moves with respect to a detector such as a nanopore.

A polynucleotide, such as a nucleic acid, is a macromolecule comprising two or more nucleotides. A polynucleotide can be single-stranded or double-stranded. A double-stranded polynucleotide is made of two single stranded polynucleotides hybridised together. The target polynucleotide can be a single-stranded polynucleotide or a double-stranded polynucleotide described in more detail herein.

A polynucleotide may comprise any combination of any nucleotides. The nucleotides can be naturally occurring or artificial.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or ribonucleic acid (RNA). The polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), bridged nucleic acid (BNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is preferably DNA, RNA or a DNA or RNA hybrid, most preferably DNA. A DNA/RNA hybrid may comprise DNA and RNA on the same strand. Preferably, the DNA/RNA hybrid comprises one DNA strand hybridized to a RNA strand.

The backbone of the polynucleotide can be altered to reduce the possibility of strand scission. For example, DNA is known to be more stable than RNA under many conditions. The backbone of the polynucleotide strand can be modified to avoid damage caused by e.g. harsh chemicals such as free radicals.

DNA or RNA that contains unnatural or modified bases can be produced by amplifying natural DNA or RNA polynucleotides in the presence of modified NTPs using an appropriate polymerase.

The nucleotides in the polynucleotide may be modified. The nucleotides may be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified with a label or a tag.

A single-stranded polynucleotide may contain regions with strong secondary structures, such as hairpins, quadruplexes, or triplex DNA. Structures of these types can be used to control the movement of the polynucleotide with respect to the nanopore. For example, secondary structures can be used to pause the movement of the polynucleotide through a nanopore, as described in more detail herein. Each successive secondary structure along the strand pauses the movement of the strand with respect to the nanopore as it is unwound and translocated. The polynucleotide may reform secondary structures after it has translocated through the nanopore. Such secondary structures can be used to prevent the polynucleotide from moving back through the nanopore under low or no applied negative voltages (applied to the trans side of the nanopore) and therefore assist in controlling the movement of the polynucleotide so it only occurs in a controlled manner in the relevant steps of the methods provided herein.

As used herein, a double stranded polynucleotide may comprise single stranded regions and regions with other structures, such as hairpin loops, triplexes and/or quadruplexes. Such secondary structures can be useful as described above in the context of single-stranded polynucleotides.

In some embodiments the target polynucleotide is a double-stranded polynucleotide. In some embodiments, prior to step (i) the target polynucleotide is comprised in or consists of a first strand of a double-stranded polynucleotide comprising said first strand and a second strand.

In some embodiments the target polynucleotide is a double-stranded polynucleotide and the first strand is hybridised to the second strand. In some embodiments the portion of the first strand between the motor protein and the second end is hybridised to the second strand. In some embodiments the motor protein is bound to the leader at the first end of the target polynucleotide and the portion of the first strand between the motor protein and the second end is hybridised to the second strand. In some embodiments (e.g. during the methods when the motor protein is used to control the movement of the target polynucleotide) the motor protein may be bound to the target polynucleotide and the portion of the first strand between the motor protein and the second end is hybridised to the second strand.

Accordingly, in some embodiments the portion of the target polynucleotide “above” the motor protein (i.e. between the motor protein and the second end of the polynucleotide) is hybridised (e.g. as a polynucleotide duplex) and the portion of the target polynucleotide “below” the motor protein (i.e. between the motor protein and the leader) is not hybridised.

In some embodiments movement of the double-stranded polynucleotide alters the portion of the double-stranded polynucleotide which is hybridised. Thus, the motor protein and/or detector (e.g. a nanopore) may act determine the portion of the polynucleotide which is hybridised as a duplex and the portion which is dehybridised.

In some embodiments movement of the target polynucleotide in the direction from the first opening of the detector to the second opening of the detector comprises separation of the first strand from the second strand. The separation of the first strand from the second strand may comprise dehybridisation of the first strand from the second strand. In some embodiments the first strand dehybridises from the second strand as the first strand moves through the polynucleotide binding site of the motor protein. Thus, the motor protein acts to unzip the double-stranded polynucleotide as the first strand moves through the polynucleotide binding site of the motor protein in the direction from the first opening to the second opening of the detector.

In some embodiments movement of the target polynucleotide in the direction from the second opening of the detector to the first opening of the detector comprises annealing of the first strand to the second strand. The annealing of the first strand to the second strand may comprise hybridisation of the first strand to the second strand. In some embodiments the first strand hybridises to the second strand as the first strand moves through the polynucleotide binding site of the motor protein in the direction from the second opening to the first opening of the detector. Thus, the separated (dehybridised) strands of the polynucleotide are re-zipped together.

The two strands of a double-stranded molecule may be attached together. For example, in embodiments wherein the target polynucleotide comprises a first strand and a second strand, the first strand may be attached to the second strand. The two strands may be covalently linked, for example at the ends of the molecules by joining the 5′ end of one strand to the 3′ end of the other with a hairpin structure.

The target polynucleotide can be any length. For example, the target polynucleotide can be at least 10, at least 50, at least 100, at least 150, at least 200, at least 250, at least 300, at least 400 or at least 500 nucleotides or nucleotide pairs in length. The target polynucleotide can be 1000 or more nucleotides or nucleotide pairs, 5000 or more nucleotides or nucleotide pairs in length or 100000 or more nucleotides or nucleotide pairs in length or 500,000 or more nucleotides or nucleotide pairs in length, or 1,000,000 or more nucleotides or nucleotide pairs in length, 10,000,000 or more nucleotides or nucleotide pairs in length, or 100,000,000 or more nucleotides or nucleotide pairs in length, or 200,000,000 or more nucleotides or nucleotide pairs in length, or the entire length of a chromosome.

The target polynucleotide may be an oligonucleotide. Oligonucleotides are short nucleotide polymers which typically have 50 or fewer nucleotides, such 40 or fewer, 30 or fewer, 20 or fewer, 10 or fewer or 5 or fewer nucleotides. The target oligonucleotide is preferably from about 15 to about 30 nucleotides in length, such as from about 20 to about 25 nucleotides in length. For example, the oligonucleotide can be about 15, about 16, about 17, about 18, about 19, about 20, about 21, about 22, about 23, about 24, about 25, about 26, about 27, about 28, about 29 or about 30 nucleotides in length.

The target polynucleotide may be a fragment of a longer polynucleotide. In this embodiment, the longer polynucleotide is typically fragmented into multiple, such as two or more, shorter polynucleotides.

The target polynucleotide may comprise the products of a PCR reaction, genomic DNA, the products of an endonuclease digestion and/or a DNA library.

The target polynucleotide may be naturally occurring. The target polynucleotide may be secreted from cells. Alternatively, the target analyte can be an analyte that is present inside cells such that the analyte must be extracted from the cells before the method can be carried out.

The target polynucleotide may be sourced from common organisms such as viruses, bacteria, archaea, plants or animals. Such organisms may be selected or altered to adjust the sequence of the target polynucleotide, for example by adjusting the base composition, removing unwanted sequence elements, and the like. The selection and alteration of organisms in order to arrive at desired polynucleotide characteristics is routine for one of ordinary skill in the art.

The source organism for the target polynucleotide may be chosen based on desired characteristics of the sequence. Desired characteristics include the ratio of single-stranded vs double-stranded polynucleotides produced by the organism; the complexity of the sequences of polynucleotides produced by the organism, the composition of the polynucleotides produced by the organism (such as the GC composition), or the length of contiguous polynucleotide strands produced by the organism. For example, when a contiguous polynucleotide strand of around 50 kb is required, lambda phage DNA can be used. If longer contiguous strands are required, other organisms can be used to produce the polynucleotide; for example E. coli produces around 4.5 Mb of contiguous dsDNA.

The target polynucleotide is often obtained from a human or animal, e.g. from urine, lymph, saliva, mucus, seminal fluid or amniotic fluid, or from whole blood, plasma or serum. The target polynucleotide may be obtained from a plant e.g. a cereal, legume, fruit or vegetable. The target polynucleotide may comprise genomic DNA. The genomic DNA may be fragmented. The DNA may be fragmented by any suitable method. For example, methods of fragmenting DNA are known in the art, Such methods may use a transposase, such as a MuA transposase. Often the genomic DNA is not fragmented.

In some embodiments the polynucleotide is synthetic or semi-synthetic. For example, DNA or RNA may be purely synthetic, synthesised by conventional DNA synthesis methods such as phosphoramidite based chemistries. Synthetic polynucleotides subunits may be joined together by known means, such as ligation or chemical linkage, to produce longer strands. In some embodiments internal self-forming structures (e.g. hairpins, quadruplexes) can be designed into the substrate e.g. by ligating appropriate sequences. Synthetic polynucleotides can be copied and scaled up for production by means known in the art, including PCR, incorporation into bacterial factories, and the like.

In some embodiments, the polynucleotide may have a simplified nucleotide composition. In some embodiments the polynucleotide has a repeating pattern of the same subunit. For example, a repeating unit may be (AmGn)q, wherein m, n and q are positive integers. For example, m is often from 1 to 20, such as from 1 to 10 e.g. from 1 to 5, e.g. 1, 2, 3, 4 or 5. n is often from 1 to 20, such as from 1 to 10 e.g. from 1 to 5, e.g. 1, 2, 3, 4 or 5. m and n may be the same or different. q is often from 1 to about 100,000. A typical repeating unit may be for example (AAAAAAGGGGGG)q. Repeating polynucleotides can be made by many means known in the art, for example by concatenating together synthetic subunits with sticky ends that enable ligation. In some embodiments the polynucleotide may therefore be a concatenated polynucleotide. Methods of concatenating polynucleotides are described in PCT/GB2017/051493.

In some embodiments, the polynucleotide can comprise bases which contain a reactive side-chain. Any suitable reactive functional groups can be incorporated on the side chain as required. Suitable examples of reactive functional groups include click chemistry reagents. Suitable examples of click chemistry include, but are not limited to, the following:
(f) copper-free variant of the 1,3 dipolar cycloaddition reaction, where an azide reacts with an alkyne under strain, for example in a cyclooctane ring;
(g) the reaction of an oxygen nucleophile on one linker with an epoxide or aziridine reactive moiety on the other; and
(h) the Staudinger ligation, where the alkyne moiety can be replaced by an aryl phosphine, resulting in a specific reaction with the azide to give an amide bond.

### Polynucleotide Adapter

In some embodiments, the leader on which the motor protein is initially bound is comprised in a polynucleotide adapter. WO 2015/110813 describes the loading of motor proteins onto a target polynucleotide such as an adapter, and is hereby incorporated by reference in its entirety.

An adapter typically comprises a polynucleotide strand capable of being attached to the end of a target polynucleotide. The target polynucleotide is typically intended for characterisation in accordance with methods disclosed herein.

A polynucleotide adapter may be added to both ends of the target polynucleotide. Alternatively, different adapters may be added to the two ends of the target polynucleotide. An adapter may be added to just one end of the target polynucleotide. Methods of adding adapters to polynucleotides are known in the art. Adapters may be attached to polynucleotides, for example, by ligation, by click chemistry, by tagmentation, by topoisomerisation or by any other suitable method.

An adapter may be synthetic or artificial. Typically, an adapter comprises a polymer as described herein. In some embodiments, the adapter comprises a polynucleotide. In some embodiments an adapter may comprise a single-stranded polynucleotide strand. In some embodiments an adapter may comprise a double-stranded polynucleotide. A polynucleotide adapter may comprise DNA, RNA, modified DNA (such as a basic DNA), RNA, PNA, LNA, BNA and/or PEG. Usually, the adapter comprises single stranded and/or double stranded DNA or RNA.

An adapter may comprise a stalling moiety as described herein. The adapter may comprise a loading site for a motor protein or polynucleotide binding protein. The adapter may comprise a tag.

An adapter may be a Y adapter. A Y adapter is typically double stranded and comprises (a) at one end, a region where the two strands are hybridised together and (b), at the other end, a region where the two strands are not complementary. The non-complementary parts of the strands form overhangs. The hybridised stem of the adapter typically attaches to the 5′ end of a first strand of a double-stranded polynucleotide and the 3′ end of a second strand of a double-stranded polynucleotide; or to the 3′ end of a first strand of a double-stranded polynucleotide and the 5′ end of a second strand of a double-stranded polynucleotide. The presence of a non-complementary region in the Y adapter gives the adapter its Y shape since the two strands typically do not hybridise to each other unlike the double stranded portion. A motor protein or polynucleotide binding protein may bind to an overhang of an adapter such as a Y adapter. In another embodiment, a motor protein or polynucleotide binding protein may bind to the double stranded region. In other embodiments, a motor protein or polynucleotide binding protein may bind to a single-stranded and/or a double-stranded region of the adapter. In other embodiments, a first motor protein or polynucleotide binding protein may bind to the single-stranded region of such an adapter and a second motor protein or polynucleotide binding protein may bind to the double-stranded region of the adapter. In some embodiments, one of the non-complementary strands of a polynucleotide adapter such as a Y adapter may provide a leader as described herein, which when contacted with a transmembrane pore is capable of threading into a nanopore.

In one embodiment the adapter comprises a membrane anchor or a pore anchor. In some embodiments, the anchor may be attached to a polynucleotide that is complementary to and hence that is hybridised to the overhang to which a motor protein or polynucleotide binding protein is bound.

In one embodiment, a polynucleotide adapter is a hairpin loop adapter, described in more detail herein. A hairpin loop adapter is an adapter comprising a single polynucleotide strand, wherein the ends of the polynucleotide strand are capable of hybridising to each other, or are hybridized to each other, and wherein the middle section of the polynucleotide forms a loop. Suitable hairpin loop adapters can be designed using methods known in the art. Typically, the 3′ end of a hairpin loop adapter attaches to the 5′ end of a first strand of a double-stranded polynucleotide and the 5′ end of the hairpin loop adapter attaches to the 3′ end of a second strand of a double-stranded polynucleotide; or the 5′ end of a hairpin loop adapter attaches to the 3′ end of a first strand of a double-stranded polynucleotide and the 3′ end of the hairpin loop adapter attaches to the 5′ end of a second strand of a double-stranded polynucleotide.

Those skilled in the art will also appreciate that when the adapter comprises a polynucleotide strand, the sequence of the adapter is typically not determinative and can be controlled or chosen according to the motor protein and other experimental conditions such as any polynucleotide to be characterised. Exemplary sequences are provided solely by way of illustration in the examples. For example, the adapter may comprise a sequence such as one or more of SEQ ID NOs: 10/64, 11-15, 17/65, or 18-22, or a polynucleotide sequence having at least 20%, such as at least 30%, e.g. at least 40% such as at least 50%, e.g. at least 60% such as at least 70%, e.g. at least 80%, for example at least 90% e.g. at least 95% sequence similarity or identity to one or more of SEQ ID NOs: 10/64, 11-15, 17/65, or 18-22. The sequence of the adapter can typically be altered without negatively affecting the efficacy of the methods provided herein.

In some embodiments a polynucleotide adapter may comprise a loading site for loading the motor protein and/or polynucleotide binding protein. The loading site may be for instance a single-stranded region which can targeted by the motor protein or polynucleotide binding protein. The loading site may be a region of the polynucleotide adapter to which a exogenous polynucleotide strand comprising the motor protein or polynucleotide binding protein can bind in order to transfer the motor protein or polynucleotide binding protein to the polynucleotide to be assessed in the methods provided herein.

In some embodiments a polynucleotide adapter comprises a double-stranded polynucleotide region for binding to an end of a target polynucleotide (e.g. a target double-stranded polynucleotide); a single-stranded polynucleotide region capable of binding a motor protein or polynucleotide binding protein (and/or having a motor protein or polynucleotide binding protein bound thereto e.g. by being stalled thereon), said region optionally comprising one or more stalling moieties as described herein; an optional further polynucleotide region optionally being attached (e.g. hybridised) to a further polynucleotide strand providing a tether to a membrane anchor as described herein; and a leader. In some embodiments the tether to the membrane anchor is not complementary to the leader such that the adapter defines a Y adapter.

### Blocking Moiety

In some embodiments a blocking moiety may be used to prevent the motor protein from disengaging from the target polynucleotide.

In some embodiments the blocking moiety is comprised in the target polynucleotide. In some embodiments the blocking moiety is comprised in a polynucleotide adapter attached to the target polynucleotide. In some embodiments a polynucleotide adapter, e.g. a polynucleotide adapter as described herein comprises a blocking moiety. In some embodiments the blocking moiety is for preventing the motor protein from disengaging from the polynucleotide.

A blocking moiety may be used to prevent the motor protein from disengaging from the target polynucleotide. The blocking moiety is typically present at the opposite end of the target polynucleotide from the leader. For example, if the leader is attached to the 3′ end of a polynucleotide strand in the target polynucleotide, the blocking moiety is typically positioned between the motor protein and the 5′ terminus of the strand. If the leader is present at the 5′ end of a polynucleotide strand in the target polynucleotide, the blocking moiety is typically positioned between the motor protein and the 3′ terminus of the strand.

For example, in some embodiments the target polynucleotide has a leader attached thereto at a first end and the second end of the target polynucleotide comprises a blocking moiety to prevent the motor protein from disengaging from the polynucleotide.

In some embodiments the target polynucleotide comprises a leader sequence at a first end of the target polynucleotide and the motor protein is bound to (e.g. stalled at) the leader; and the blocking moiety is positioned between the motor protein and the second end of the polynucleotide (i.e. the terminus of the polynucleotide at the second end of the polynucleotide) thereby preventing the motor protein from disengaging from the target polynucleotide at the second end of the target polynucleotide.

For example, in some embodiments the target polynucleotide comprises a leader at the 5′ end of a first strand and the motor protein is stalled on the leader; and the blocking moiety is positioned between the motor protein and the 3′ terminus of the first strand of the polynucleotide thereby preventing the motor protein from disengaging from the target polynucleotide at the 3′ end of the first strand of the target polynucleotide. In other embodiments the target polynucleotide comprises a leader sequence at the 3′ end of a first strand and the motor protein is stalled on the leader; and the blocking moiety is positioned between the motor protein and the 5′ terminus of the first strand of the polynucleotide thereby preventing the motor protein from disengaging from the target polynucleotide at the 5′ end of the first strand of the target polynucleotide. The leader may be comprised in an adapter as described herein.

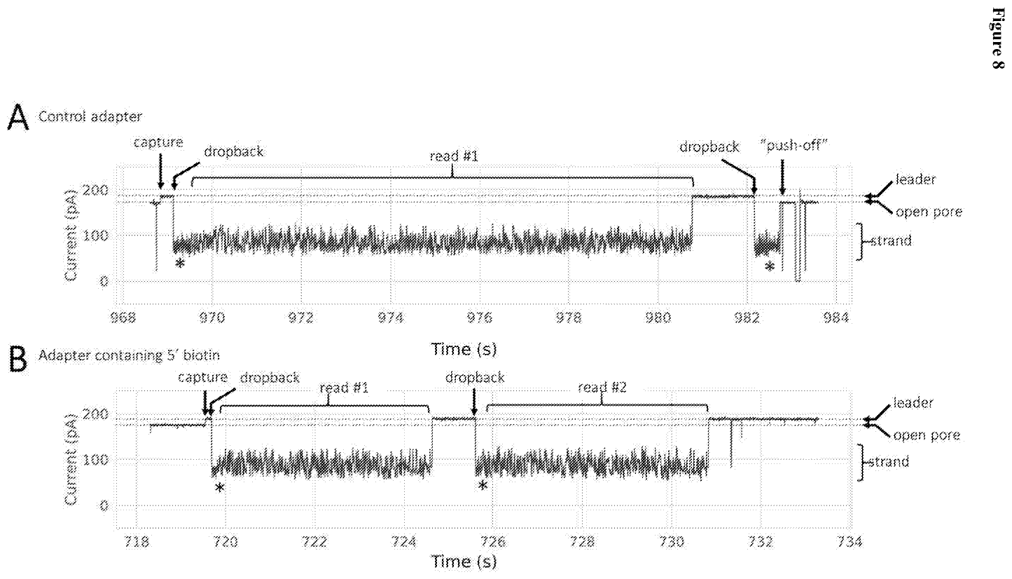

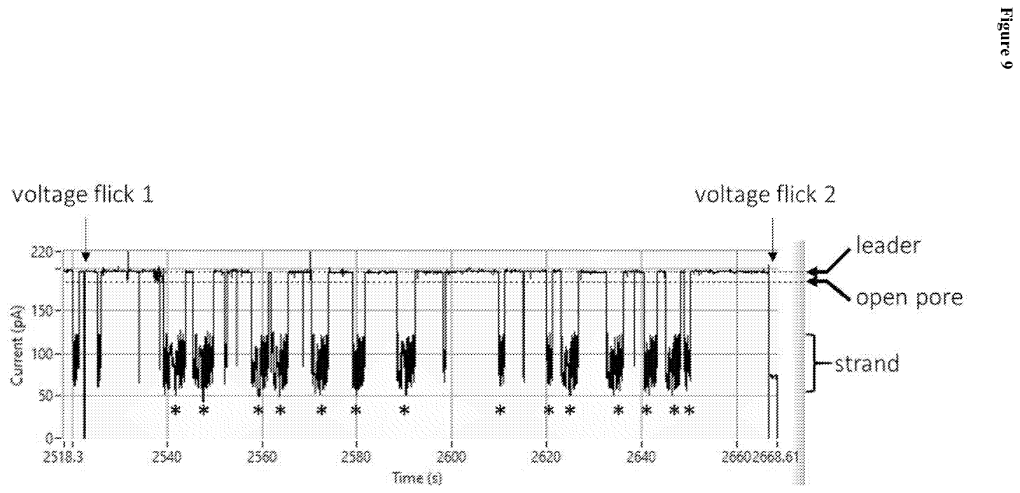

When the target polynucleotide is a double-stranded polynucleotide the blocking moiety is typically positioned on the same strand as the motor protein. If a motor protein is present on each strand of the double-stranded polynucleotide (e.g. when the double-stranded polynucleotide is rotationally symmetrical) a blocking moiety is typically present on each strand of the polynucleotide. An example of this is shown in FIG. 5.

However in some embodiments the target polynucleotide comprises a double-stranded polynucleotide e.g, wherein the first and second strands are attached together e.g. with a hairpin adapter as described herein. In such embodiments the leader may be present at the first end of the first strand, the second end of the first strand may be attached to the first end of the second strand and the blocking moiety may be present at the second end of the second strand. An example of this is shown in FIG. 4.

The blocking moiety is typically too large to pass through the motor protein (e.g. through the polynucleotide-binding site of a motor protein which has been modified to topologically close the polynucleotide binding site around the target polynucleotide) and so when the movement of the polynucleotide with respect to the detector brings the blocking moiety into contact with the motor protein (e.g. when the target polynucleotide is unbound from the polynucleotide binding site of the motor protein), the further movement of the polynucleotide through the motor protein and therefore typically also through the detector is prevented. Accordingly, in some embodiments, the blocking moiety limits the movement of the target polynucleotide through the polynucleotide binding site of the motor protein and thereby limits the movement of the polynucleotide in the second direction with respect to the detector. At such time the motor protein may rebind to the polynucleotide. The polynucleotide may then move through the pore in the opposite direction under the control of the motor protein.

Any suitable blocking moiety can be used in the provided methods. Suitable blocking moieties include many of the same groups that can be used as pausing moieties as described herein. For example, a blocking moiety may comprise one or more of:
a polynucleotide secondary structure, preferably a hairpin or G-quadruplex (TBA);
a nucleic acid analog, preferably selected from peptide nucleic acid (PNA), glycerol nucleic acid (GNA), threose nucleic acid (TNA), locked nucleic acid (LNA), bridged nucleic acid (BNA) and abasic nucleotides;
fluorophores, avidins such as traptavidin, streptavidin and neutravidin, and/or biotin, cholesterol, methylene blue, dinitrophenols (DNPs), digoxigenin and/or anti-digoxigenin and dibenzylcyclooctyne groups; and
a polynucleotide binding protein.

The blocking moiety may be attached to the polynucleotide in any suitable manner. The blocking moiety may be attached directly to the polynucleotide. The blocking moiety may be attached to the polynucleotide via a linker. Any suitable linker may be used. Any suitable chemistry for attaching the blocking moiety to the polynucleotide may be used. Any of the attachment methods described herein for attaching the leader to the polynucleotide may be used to attach the blocking moiety to the polynucleotide. The attachment means may be the same or different. In some embodiments a linker is used to attach the leader to the polynucleotide and the blocking moiety is attached directly to the polynucleotide. In some embodiments the leader is attached directly to the polynucleotide and a linker is used to attach the blocking moiety to the polynucleotide. In some embodiments the leader is attached directly to the polynucleotide and the blocking moiety is attached directly to the polynucleotide. In some embodiments a linker is used to attach the leader to the polynucleotide and a linker is used to attach the blocking moiety to the polynucleotide. When a linker is used to attach both the blocking moiety and the leader to the polynucleotide the linkers may be the same or different.

### Spacers

In some embodiments, a polynucleotide or polynucleotide adapter may comprise one or more spacers, e.g. from one to about 10 spacers, e.g. from 1 to about 5 spacers, e.g. 1, 2, 3, 4 or 5 spacers. The spacer may comprise any suitable number of spacer units. A spacer typically provides an energy barrier which impedes movement of a polynucleotide binding protein. For example, a spacer may impede movement of a motor protein or polynucleotide binding protein by reducing the traction of the protein, e.g. using an abasic spacer. A spacer may physically block movement of the protein, for instance by introducing a bulky chemical group to physically impede the movement of the polynucleotide binding protein.

In some embodiments, one or more spacers are included in the polynucleotide or in a polynucleotide adapter to provide a distinctive signal when they pass through or across a nanopore. One or more spacers may be used to define or separate one or more regions of a polynucleotide; e.g. to separate an adapter from the target polynucleotide.

In some embodiments, a spacer may comprise a linear molecule, such as a polymer, e.g. a polypeptide or a polyethylene glycol (PEG). Typically, such a spacer has a different structure from the target polynucleotide. For instance, if the target polynucleotide is DNA, the or each spacer typically does not comprise DNA. In particular, if the target polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

In some embodiments, a spacer may comprise one or more chemical groups, e.g. one or more pendant chemical groups. The one or more chemical groups may be attached to one or more nucleobases in a polynucleotide adapter. The one or more chemical groups may be attached to the backbone of a polynucleotide adapter. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

Suitable spacers can be designed or selected depending on the nature of the polynucleotide or polynucleotide adapter, the motor protein and the conditions under which the method is to be carried out.

### Tags

In some embodiments a polynucleotide or polynucleotide adapter may comprise a tag or tether. For example, a polynucleotide can bind to a tag on a nanopore, e.g., via its adaptor, and release at some point, e.g., during characterization of the polynucleotide by the nanopore. A strong non-covalent bond (e.g., biotin/avidin) is still reversible and can be useful in some embodiments of the methods described herein.

In some embodiments, the pair of pore tag and polynucleotide adaptor can be configured such that the binding strength or affinity of a binding site on the polynucleotide (e.g., a binding site provided by an anchor or a leader sequence of an adaptor or by a capture sequence within the duplex stem of an adaptor) to a tag on a nanopore is sufficient to maintain the coupling between the nanopore and polynucleotide until an applied force is placed on it to release the bound polynucleotide from the nanopore.

In some embodiments, the tags or tethers are uncharged. This can ensure that the tags or tethers are not drawn into the nanopore under the influence of a potential difference.

One or more molecules that attract or bind the polynucleotide or adaptor may be linked to the detector (e.g. the pore). Any molecule that hybridizes to the adaptor and/or target polynucleotide may be used. The molecule attached to the pore may be selected from a PNA tag, a PEG linker, a short oligonucleotide, a positively charged amino acid and an aptamer. Pores having such molecules linked to them are known in the art. For example, pores having short oligonucleotides attached thereto are disclosed in Howarka et al (2001) Nature Biotech. 19:636-639 and WO 2010/086620, and pores comprising PEG attached within the lumen of the pore are disclosed in Howarka et al (2000) J. Am. Chem. Soc. 122 (11): 2411-2416.

A short oligonucleotide attached to the detector (e.g. a transmembrane pore), which oligonucleotide comprises a sequence complementary to a sequence in the leader sequence or another single stranded sequence in the adaptor may be used to enhance capture of the target polynucleotide in the methods described herein.

In some embodiments, the tag or tether may comprise or be an oligonucleotide (e.g., DNA, RNA, LNA, BNA, PNA, or morpholino). The oligonucleotide (e.g., DNA, RNA, LNA, BNA, PNA, or morpholino) can have about 10-30 nucleotides in length or about 10-20 nucleotides in length. In some embodiments, the oligonucleotide (e.g., DNA, RNA, LNA, BNA, PNA, or morpholino) for use in the tag or tether can have at least one end (e.g., 3′- or 5′-end) modified for conjugation to other modifications or to a solid substrate surface including, e.g., a bead. The end modifiers may add a reactive functional group which can be used for conjugation. Examples of functional groups that can be added include, but are not limited to amino, carboxyl, thiol, maleimide, aminooxy, and any combinations thereof. The functional groups can be combined with different length of spacers (e.g., C3, C9, C12, Spacer 9 and 18) to add physical distance of the functional group from the end of the oligonucleotide sequence.

In some embodiments, the tag or tether may comprise or be a morpholino oligonucleotide. The morpholino oligonucleotide can have about 10-30 nucleotides in length or about 10-20 nucleotides in length. The morpholino oligonucleotides can be modified or unmodified. For example, in some embodiments, the morpholino oligonucleotide can be modified on the 3′ and/or 5′ ends of the oligonucleotides. Examples of modifications on the 3′ and/or 5′ end of the morpholino oligonucleotides include, but are not limited to 3′ affinity tag and functional groups for chemical linkage (including, e.g., 3′-biotin, 3′-primary amine, 3′-disulfide amide, 3′-pyridyl dithio, and any combinations thereof); 5′ end modifications (including, e.g., 5′-primary ammine, and/or 5′-dabcyl), modifications for click chemistry (including, e.g., 3′-azide, 3′-alkyne, 5′-azide, 5′-alkyne), and any combinations thereof.

In some embodiments, the tag or tether may further comprise a polymeric linker, e.g., to facilitate coupling to a detector e.g. a nanopore. An exemplary polymeric linker includes, but is not limited to polyethylene glycol (PEG). The polymeric linker may have a molecular weight of about 500 Da to about 10 kDa (inclusive), or about 1 kDa to about 5 kDa (inclusive). The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof. In some embodiments, the tag or tether may further comprise a 1 kDa PEG with a 5′-maleimide group and a 3′-DBCO group. In some embodiments, the tag or tether may further comprise a 2 kDa PEG with a 5′-maleimide group and a 3′-DBCO group. In some embodiments, the tag or tether may further comprise a 3 kDa PEG with a 5′-maleimide group and a 3′-DBCO group. In some embodiments, the tag or tether may further comprise a 5 kDa PEG with a 5′-maleimide group and a 3′-DBCO group.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of a nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444.

In some embodiments where one or more tags or tethers are attached to a nanopore via cysteine linkage(s), the one or more cysteines can be introduced to one or more monomers that form the nanopore by substitution. In some embodiments, the nanopore may be chemically modified by attachment of (i) Maleimides including diabromomaleimides such as: 4-phenylazomaleinanil, 1.N-(2-Hydroxyethyl) maleimide, N-Cyclohexylmaleimide, 1.3-Maleimidopropionic Acid, 1.1-4-Aminophenyl-1H-pyrrole, 2,5,dione, 1.1-4-Hydroxyphenyl-1H-pyrrole, 2,5,dione, N-Ethylmaleimide, N-Methoxycarbonylmaleimide, N-tert-Butylmaleimide, N-(2-Aminoethyl) maleimide, 3-Maleimido-PROXYL, N-(4-Chlorophenyl) maleimide, 1-[4-(dimethylamino)-3,5-dinitrophenyl]-1H-pyrrole-2,5-dione, N-[4-(2-Benzimidazolyl)phenyl] maleimide, N-[4-(2-benzoxazolyl)phenyl] maleimide, N-(1-naphthyl)-maleimide, N-(2,4-xylyl) maleimide, N-(2,4-difluorophenyl) maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl-1-[2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC=2C═CN═CC2, SMILES O=C1C—CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl) maleimide, N-(4-nitrophenyl) maleimide (ii) Iodocetamides such as: 3-(2-Iodoacetamido)-proxyl, N-(cyclopropylmethyl)-2-iodoacetamide, 2-iodo-N-(2-phenylethyl) acetamide, 2-iodo-N-(2,2,2-trifluoroethyl) acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl) acetamide, 2-bromo-N-(3-(trifluoromethyl)phenyl) acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide, 2-adamantan-1-yl-2-bromo-N-cyclohexyl-acetamide, 2-bromo-N-(2-methylphenyl) butanamide, Monobromoacetanilide, (iv) Disulphides such as: aldrithiol-2, aldrithiol-4, isopropyl disulfide, 1-(Isobutyldisulfanyl)-2-methylpropane, Dibenzyl disulfide, 4-aminophenyl disulfide, 3-(2-Pyridyldithio) propionic acid, 3-(2-Pyridyldithio) propionic acid hydrazide, 3-(2-Pyridyldithio) propionic acid N-succinimidyl ester, am6amPDP1-βCD and (v) Thiols such as: 4-Phenylthiazole-2-thiol, Purpald, 5,6,7,8-tetrahydro-quinazoline-2-thiol.

In some embodiments, the tag or tether may be attached directly to a nanopore or via one or more linkers. The tag or tether may be attached to the nanopore using the hybridization linkers described in WO 2010/086602. Alternatively, peptide linkers may be used. Peptide linkers are amino acid sequences. The length, flexibility and hydrophilicity of the peptide linker are typically designed such that it does not to disturb the functions of the monomer and pore. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

### Anchor

In one embodiment, a polynucleotide or polynucleotide adapter may comprise a membrane anchor or a transmembrane pore anchor. In one embodiment the anchor assists in the characterisation of a target polynucleotide in accordance with the methods disclosed herein. For example, a membrane anchor or transmembrane pore anchor may promote localisation of the selected polynucleotides around a nanopore.

The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol. The anchor may comprise thiol, biotin or a surfactant. In one aspect the anchor may be biotin (for binding to streptavidin), amylose (for binding to maltose binding protein or a fusion protein), Ni-NTA (for binding to poly-histidine or poly-histidine tagged proteins) or peptides (such as an antigen).

In one embodiment, the anchor comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The adapter may hybridise to a complementary sequence on a circular polynucleotide linker.

The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602.

In one embodiment, the anchor is cholesterol or a fatty acyl chain. For example, any fatty acyl chain having a length of from 6 to 30 carbon atom, such as hexadecanoic acid, may be used. Examples of suitable anchors and methods of attaching anchors to adapters are disclosed in WO 2012/164270 and WO 2015/150786.

In another embodiment the anchor may consist or comprise a hydrophobic modification to the polynucleotide or polynucleotide adapter. The hydrophobic modification may comprise a modified phosphate group comprised within the polynucleotide or polynucleotide anchor. The hydrophobic modification may for example comprise a phosphorothioate such as a charge-neutralized alkyl-phosphorothioate (PPT) as described in Jones et al, J. Am. Chem. Soc. 2021, 143, 22, 8305, the entire contents of which are hereby incorporated by reference. Suitable alkyl groups include for example C1-C10 alkyl groups such as C2-C₆ alkyl groups; e.g. methyl, ethyl, propyl, butyl, pentyl and hexyl groups. Incorporation of the charge-neutralized alkyl-phosphorothioate into a polynucleotide allows for the polynucleotide to anchor to a hydrophobic region such as a lipid bilayer.

### Detector

In the methods provided herein, the polynucleotide is moved with respect to a detector such as a nanopore. The detector may be selected from (i) a zero-mode waveguide, (ii) a field-effect transistor, optionally a nanowire field-effect transistor; (iii) an AFM tip; (iv) a nanotube, optionally a carbon nanotube; and (v) a nanopore. Preferably, the detector is a nanopore.

The polynucleotide may be characterised in the methods provided herein in any suitable manner. In one embodiment the polynucleotide is characterised by detecting an ionic current or optical signal as the polynucleotide moves with respect to a nanopore. This is described in more detail herein. The method is amenable to these and other methods of detecting polynucleotides.

In another non-limiting example, in one embodiment the polynucleotide is characterised by detecting the by-products of a polynucleotide-processing reaction, such as a sequencing by synthesis reaction. The method may thus involve detecting the product of the sequential addition of (poly) nucleotides by an enzyme such as a polymerase to the nucleic acid strand. The product may be a change in one or more properties of the enzyme such as in the conformation of the enzyme. Such methods may thus comprise subjecting an enzyme such as polymerase or a reverse transcriptase to a double-stranded polynucleotide under conditions such that the template-dependent incorporation of nucleotide bases into a growing oligonucleotide strand causes conformational changes in the enzyme in response to sequentially encountering template strand nucleic acid bases and/or incorporating template-specified natural or analog bases (i.e., an incorporation event), detecting the conformational changes in the enzyme in response to such incorporation events, and thereby detecting the sequence of the template strand. In such methods the polynucleotide strand may be moved in accordance with the methods provided herein. Such methods may involve detecting and/or measuring incorporation events using methods known to those skilled in the art, such as those described in US 2017/0044605.

In another embodiment, by-products may be labelled so that a phosphate labelled species is released upon the addition of a nucleotide to a synthesised nucleic acid strand that is complementary to the template strand, and the phosphate labelled species is detected e.g. using a detector as described herein. The polynucleotide being characterised in this way may be moved in accordance with the methods herein. Suitable labels may be optical labels that are detected using a nanopore, or a zero mode wave guide, or by Raman spectroscopy, or other detectors. Suitable labels may be non-optical labels that are detected using a nanopore, or other detectors.

In another approach, nucleoside phosphates (nucleotides) are not labelled and upon the addition of a nucleotide to a synthesised nucleic acid strand that is complementary to the template strand, a natural by-product species is detected. Suitable detectors may be ion-sensitive field-effect transistors, or other detectors.

These and other detection methods are suitable for use in the methods described herein. Any suitable measurements can be taken using a detector as the polynucleotide moves with respect to the detector.

### Nanopore

In embodiments of the disclosed methods wherein the detector is a nanopore, any suitable nanopore can be used. In one embodiment a nanopore is a transmembrane pore.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

In the methods provided herein, the nanopore typically has a first opening and a second opening. The first opening is typically the cis opening and the second opening is typically the trans opening. However in some embodiments the first opening is the trans opening and the second opening is the cis opening. The motor protein used in the methods provided herein is typically provided at the first opening of the nanopore and thus controls the movement of the target polynucleotide in the direction from the second opening of the nanopore towards the first opening of the nanopore.

Any transmembrane pore may be used in the methods provided herein. The pore may be biological or artificial. Suitable pores include, but are not limited to, protein pores, polynucleotide pores and solid state pores.

A solid state pore may, in one embodiment, comprise a nanochannel. In some embodiments the solid state pore is a pore disclosed in WO 2003/003446, WO 2009/020682 or WO 2016/187519, each of which is incorporated by reference in their entirety.

In one embodiment, the pore may be a DNA origami pore (Langecker et al., Science, 2012; 338:932-936). Suitable DNA origami pores are disclosed in WO2013/083983, WO 2018/011603 and WO 2020/025974, each of which is incorporated by reference in their entirety.

In one embodiment, the nanopore is a scaffolded polypeptide nanopore. In some embodiments the pore is a scaffolded polypeptide nanopore as disclosed in WO 2020/025909 or WO 2020/074399, each of which is incorporated by reference in their entirety.

In one embodiment, the nanopore is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, the transmembrane protein pore comprises a barrel or channel through which the ions may flow. The subunits of the pore typically surround a central axis and contribute strands to a transmembrane B-barrel or channel or a transmembrane «-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, the nanopore is a transmembrane protein pore derived from β-barrel pores or α-helix bundle pores. β-barrel pores comprise a barrel or channel that is formed from β-strands. Suitable B-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and α outer membrane proteins, such as WZA and ClyA toxin.

In one embodiment the nanopore is a transmembrane pore derived from or based on Msp, α-hemolysin (α-HL), lysenin, CsgG, ClyA, Sp1 or haemolytic protein fragaceatoxin C (FraC).

In one embodiment, the nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591, WO 2017/149316, WO 2017/149317, WO 2017/149318 and WO 2019/002893, each of which is hereby incorporated by reference in its entirety.

In one embodiment, the nanopore is a transmembrane pore derived from lysenin. Examples of suitable pores derived from lysenin are disclosed in WO 2013/153359, which is hereby incorporated by reference in its entirety.

In one embodiment, the nanopore is a transmembrane pore derived from or based on a-hemolysin (a-HL). The wild type a-hemolysin pore is formed of 7 identical monomers or sub-units (i.e., it is heptameric). An a-hemolysin pore may be a-hemolysin-NN or a variant thereof. The variant preferably comprises N residues at positions E111 and K147.

In one embodiment, the nanopore is a transmembrane protein pore derived from Msp, e.g. from MspA. Examples of suitable pores derived from MspA are disclosed in WO 2012/107778.

In one embodiment, the nanopore is a transmembrane pore derived from or based on ClyA. Examples of suitable pores derived from ClyA are disclosed in Soskine et al., Nano Letters 2012 12 (9), 4895-4900; WO 2014/153625; and WO 2017/098322, each of which is hereby incorporated by reference.

In one embodiment, the nanopore is a transmembrane pore derived from Phi29. Examples of suitable pores derived from Phi29 are disclosed in Wendell et al., Nature Nanotech 4, 765-772 (2009), WO 2010/062697, WO 2019/157365 and WO 2019/157424, each of which is hereby incorporated by reference.

In some embodiments the nanopore is selected from M-ring protein, perforin-2, PlyAB (pleurotolysin), SpoIIIAG, VirB7, Type II secretion system protein D, GspD, InvG, PilQ, pentraxin, and portal proteins including T4, T7, P23_45, G20c and Phi29 nanopores.

In one embodiment, the nanopore is a transmembrane pore derived from or based on a Rhodococcus species of bacteria, for example Rhodococcus corynebacteroides or Rhodococcus ruber, for example PorARr, PorBRr or PorARc. Examples of such pores are described in Piselli et al., Eur Biophys J 51, 309-323 (2022).

### Membrane

In the disclosed methods, the detector is typically a nanopore present in a membrane. Any suitable membrane may be used.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

In some embodiments, the membrane is one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69:3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In some embodiments, a lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy (Polyethylene glycol)-2000]; functionalised PEG Lipids, such as 1,2-Distearoyl-sn-Glycero-3 Phosphoethanolamine-N-[Biotinyl(Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein are typically carried out using (i) an artificial amphiphilic layer comprising a pore, (ii) an isolated, naturally-occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### General Methods

As mentioned above, the methods provided herein may be operated using any suitable detector, and as such any suitable apparatus for detecting polynucleotides can be used.

The methods provided herein may in some embodiments be carried out using any apparatus that is suitable for transmembrane pore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The methods may be carried out using the apparatus described in WO 2008/102120, WO 2010/122293 or WO 00/28312. In brief, the binding of a molecule (e.g. a target polynucleotide) in the channel of a pore will have an effect on the open-channel ion flow through the pore, which is the essence of “molecular sensing” of pore channels. Variation in the open-channel ion flow can be measured using suitable measurement techniques by the change in electrical current. The degree of reduction in ion flow, as measured by the reduction in electrical current, is related to the size of the obstruction within, or in the vicinity of, the pore. Binding of a molecule of interest (e.g. the target polynucleotide) in or near the pore therefore provides a detectable and measurable event, thereby forming the basis of a “biological sensor”. Detecting the presence of biological molecules finds application in personalised drug development, medicine, diagnostics, life science research, environmental monitoring and in the security and/or the defence industry.

When used to characterize the polynucleotide, the presence, absence or one or more characteristics of the target polynucleotide are determined. The methods may be for determining the presence, absence or one or more characteristics of at least one target polynucleotide. The methods may concern determining the presence, absence or one or more characteristics of two or more target polynucleotide. The methods may comprise determining the presence, absence or one or more characteristics of any number of target polynucleotides, such as 2, 5, 10, 15, 20, 30, 40, 50, 100 or more target polynucleotides. Any number of characteristics of the one or more target polynucleotides may be determined, such as 1, 2, 3, 4, 5, 10 or more characteristics. Characteristics amenable to being detected in the methods provide herein include the identity or sequence of the polynucleotide, the length, of the polynucleotide, whether or not the polynucleotide is modified, etc. In some embodiments the methods provided herein are methods of sequencing a target polynucleotide. In some embodiments a polynucleotide sequence may be determined in real-time by aligning real-time signal or basecalling to known references. Exemplary methods of determining a polynucleotide sequence are described in WO 2016/059427, incorporated by reference herein.

When used to characterize the polynucleotide, the methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The methods may involve measuring an optical signal as described in Chen et al, Nature Communications (2018) 9:1733, the entire contents of which are hereby incorporated by reference. For example, a nanopore such as an optically engineered nanopore structure (e.g. a plasmonic nanoslit) may be used to locally enable single-molecule surface enhanced Raman spectroscopy (SERS) to allow the characterisation of the polynucleotide through direct Raman spectroscopic detection.

The methods may be carried out on a silicon-based array of wells where each array comprises 128, 256, 512, 1024, 2000, 3000, 4000, 6000, 10000, 12000, 15000 or more wells.

The methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

In some embodiments, the methods comprise providing a condition for promoting the unbinding of the target polynucleotide from the polynucleotide binding site of the motor protein and/or for retarding re-binding of the target polynucleotide to the polynucleotide binding site of the motor protein.

The methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl2). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

In some embodiments providing said condition comprises providing a salt concentration so as to increase the rate of unbinding of the target polynucleotide from the polynucleotide binding site of the motor protein. In some embodiments providing said condition comprises providing a salt concentration so as to reduce the rate of re-binding of the target polynucleotide to the polynucleotide binding site of the motor protein. Determining a suitable salt concentration to promote unbinding of a target polynucleotide from the polynucleotide-binding site of a motor protein and/or for retarding re-binding is within the capacity of one skilled in the art in view of the disclosure herein.

In some embodiments providing said condition comprises providing an osmolarity so as to increase the rate of unbinding of the target polynucleotide from the polynucleotide binding site of the motor protein. In some embodiments providing said condition comprises providing an osmolarity so as to reduce the rate of re-binding of the target polynucleotide to the polynucleotide binding site of the motor protein. Determining a suitable osmolarity to promote unbinding of a target polynucleotide from the polynucleotide-binding site of a motor protein and/or for retarding re-binding is within the capacity of one skilled in the art in view of the disclosure herein.

The methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

In some embodiments providing said condition comprises increasing the temperature so as to increase the rate of unbinding of the target polynucleotide from the polynucleotide binding site of the motor protein. In some embodiments providing said condition comprises increasing the temperature so as to reduce the rate of re-binding of the target polynucleotide to the polynucleotide binding site of the motor protein. Without being bound in any way by theory, the inventors consider that increasing the temperature may promote re-reading by e.g. increasing the off-rate of the motor protein from the polynucleotide. Determining a suitable temperature to promote unbinding of a target polynucleotide from the polynucleotide-binding site of a motor protein and/or for retarding re-binding is within the capacity of one skilled in the art in view of the disclosure herein.

Examples of providing a condition to promote re-reading by providing a temperature for promoting re-reading are provided herein, e.g. see Example 4. In some embodiments, providing a condition for promoting the unbinding of the target polynucleotide from the polynucleotide binding site of the motor protein and/or for retarding re-binding of the target polynucleotide to the polynucleotide binding site of the motor protein may comprise providing a temperature of from about 20° C. to about 50° C. such as from about 30° C. to about 45° C. e.g. from about 34° C. to about 40° C., e.g. about 31, 32, 33, 34, 35, 36, 37, 38, or 39° C.

### Polynucleotide Adapters

Also provided are polynucleotide adapters comprising motor proteins. It will be understood that any of the polynucleotide adapters disclosed herein can be applied in the embodiments of the methods discussed herein and above.

In one embodiment, provided herein is a polynucleotide adapter having a first end comprising a leader and a second end comprising an attachment point for attaching to a polynucleotide analyte at a first end of the polynucleotide analyte; wherein said polynucleotide adapter comprises a motor protein stalled thereon in an orientation for processing the adapter in a direction from the second end to the first end.

In one embodiment, the polynucleotide adapter is a polynucleotide adapter as described in more detailed herein. In one embodiment, the motor protein is a motor protein as described herein.

The motor protein is oriented to process the polynucleotide adapter in the direction away from an attachment point on the adapter for attaching to a double-stranded polynucleotide, i.e. towards the leader. The motor protein may be orientated on the polynucleotide adapter to control the movement of the target polynucleotide in the trans-to-cis direction. The motor protein may be oriented on the polynucleotide adapter to control the movement of the target polynucleotide with respect to a detector such as a nanopore in a direction towards the motor protein; i.e., out of the detector e.g. out of the nanopore as described in more detail herein.

In some embodiments the polynucleotide adapter comprises a stalling moiety as described herein. In some embodiments the polynucleotide adapter comprises a pausing moiety as described herein.

### Kit

Also provided are kits comprising polynucleotide adapters and motor proteins. It will be understood that any of the polynucleotide adapters disclosed herein can be applied in the embodiments of the kits discussed herein and above.

In some embodiments provided herein is a kit, comprising a first adapter having a first end comprising a leader and a second end comprising an attachment point for attaching to a polynucleotide analyte at a first end of the polynucleotide analyte; wherein said polynucleotide adapter comprises a motor protein stalled thereon in an orientation for processing the adapter in a direction from the second end to the first end; and a second adapter comprising (i) an attachment point for attaching to a polynucleotide analyte at a second end of the polynucleotide analyte; and (ii) a blocking moiety suitable for preventing the motor protein of the first adapter from disengaging from the polynucleotide analyte when the first adapter is attached to the polynucleotide analyte.

### System

Also provided are systems comprising polynucleotide adapters, motor proteins, and nanopores. It will be understood that any of the polynucleotide adapters disclosed herein can be applied in the embodiments of the systems discussed herein and above.

In one embodiment provided is a system for characterising a target double-stranded polynucleotide comprising:
a polynucleotide adapter having a first end comprising a leader and a second end comprising an attachment point for attaching to a polynucleotide analyte at a first end of the polynucleotide analyte; wherein said polynucleotide adapter comprises a motor protein stalled thereon in an orientation for processing the adapter in a direction from the second end to the first end.
a nanopore for characterising the target polynucleotide as the target polynucleotide moves with respect to the nanopore; and
a motor protein for controlling the movement of the target polynucleotide with respect to the nanopore

In one embodiment, the polynucleotide adapter is a polynucleotide adapter as described in more detailed herein. In one embodiment, the motor protein is a motor protein as described herein. In one embodiment the nanopore is a nanopore as described herein. The system may further comprise a membrane; control equipment; etc as defined herein.

The system and kit disclosed herein may be configured for use with an algorithm, also provided herein, adapted to be run on a computer system. The algorithm may be adapted to detect information characteristic of a polynucleotide (e.g. characteristic of the sequence of the polynucleotide), and to selectively process the signal obtained as the polynucleotide moves with respect to a nanopore. Also provided is a system comprising computing means configured to detect information characteristic of a polynucleotide (e.g. characteristic of the sequence of the polynucleotide) and to selectively process the signal obtained as the polynucleotide moves with respect to the nanopore. In some embodiments the system comprises receiving means for receiving data from detection of the polynucleotide, processing means for processing the signal obtained as the polynucleotide moves with respect to the nanopore, and output means for outputting the characterisation information thus obtained.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

This example demonstrates the controlled translocation of a DNA polynucleotide strand through a nanopore using a DNA motor which unwinds dsDNA whilst it translocates 5′-3′ on ssDNA. The DNA motor was initially stalled on a Y-adapter ligated to the polynucleotide. The Y-adapter contained an oligonucleotide bearing a leader with thirty 3′-terminal C3 spacer residues. The polynucleotide was translocated through the nanopore in distinct phases: (1) an enzyme-free phase, in which the 3′ end of the polynucleotide was captured by the nanopore, and the nanopore translocated and separated the duplex under positive applied potential until it reaches the DNA motor stalled on the distal 5′ end; (2) a ‘de-stalling’ phase, in which the DNA motor initially could not move over the stall under positive bias but was activated (‘de-stalled’) by applying a reverse potential; (3) a DNA motor-controlled phase, in which the motor began to move the DNA 5′-3′ out of the nanopore against the applied potential; (4) upon reaching the end of the polynucleotide, a constant blockade level was seen that could be cleared by reversing the potential to eject the strand; and occasionally (5) under the force due to the applied sequencing potential, the enzyme would spontaneously slip backwards, rebind to upstream DNA, and repeat from step (3).

A Y-adapter was prepared by annealing DNA oligonucleotides (SEQ ID NOs: 17/65, SEQ ID NO: 22, SEQ ID NO: 19 and SEQ ID NO: 21). A DNA motor (Dda helicase) was loaded onto the adapter. The SEQ ID NO: 22 oligonucleotide contained the C₃ spacer residues described above.

A seven-fragment DNA library was derived via digest of bacteriophage lambda DNA using SnaBI and BamHI restriction enzymes, and end-repaired and dA-tailed by NEBNext end repair and NEBNext dA-tailing modules (New England Biolabs (NEB)) to generate 3′ dA overhangs at both ends of each fragment.

The seven-fragment DNA library was ligated to the dA-tailed end of the Y-adapter using LNB from Oxford Nanopore Technologies sequencing kit (LSK-SQK109) and T4 DNA Ligase (NEB). The sample was purified using Agencourt AMPure XP (Beckman Coulter) beads, with two washes with LFB from Oxford Nanopore Technologies sequencing kit (LSK-SQK109). The ligated substrate was eluted into 10 mM Tris-C1, 50 mM NaCl (pH 8.0), yielding a ‘DNA library’.

Electrical measurements were acquired on a FLO-MIN106 MinION flow cell and MinION Mk1b from Oxford Nanopore Technologies. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

The DNA library was run with a custom sequencing script to control the applied potential as follows: 55 sec capture phase (+120 mV); 5 sec de-stalling phase (−20 mV); 55 seconds sequencing (+120 mV); eject phase (0 mV, 1 sec; −120 mV, 3 sec). This sequence of applied potentials was repeated multiple times. Raw data was collected in a bulk FAST5 file using MinKNOW software (Oxford Nanopore Technologies).

FIG. 6a shows a schematic of the experiment. The experiment includes a ‘rereading’ step (RR), wherein the enzyme unbinds and slips back from the 3′ C3 (non-DNA) leader to an earlier position on the DNA strand (E) and translocates 5′ to 3′ once more, resulting in multiple reads of the same DNA strand. The open-pore level is not seen between the re-reads, meaning it is unlikely that the molecule was ejected from the nanopore. FIG. 6b shows an example current-time trace of a molecule that was read twice (i and ii). A Hidden Markov Model was trained to map the enzyme-controlled portions against a reference for each restriction fragment (FIG. 6c). The data show that the reads map to the same fragment in the reference, and examples were recorded that partly mapped two or three times, confirming that the strand was read multiple times.

### Example 2

This example demonstrates how a native DNA analyte may be re-read multiple times using motor proteins having different linker lengths of the disulfide closure.

Y-adapters bearing a leader arm containing 30 C3 Spacer units were prepared by annealing four DNA oligonucleotides with sequences SEQ ID NOs: 50/66, SEQ ID NO: 51, SEQ ID NO: 52, and SEQ ID NO: 53. A DNA motor (a Dda helicase) was loaded onto each adapter, and the disulfide closed via reaction with one of the following linkers: diamide (TMAD), BMOE (1,2-bismaleimidoethane), BMOP (1,3-bismaleimidopropane), BMB (1,4-bismaleimidobutane), BM(PEG)2 (1,8-bismaleimido-diethyleneglycol) or BM (PEG) 3 (1,11-bismaleimido-triethyleneglycol).

E. coli K12 PCR DNA was obtained by extraction from E. coli cells using a Qiagen genomic tip kit, sheared to a ˜10 kb cutoff using a Covaris gTube, end-repaired and dA-tailed using an Ultra II end-repair and dA-tailing kit (New England Biolabs), ligated to PCR adapters (PCA; Oxford Nanopore Technologies), and PCR-amplified using LongAmp Taq. The resultant double-stranded analyte was end-repaired and dA-tailed by NEBNext end repair and NEBNext dA-tailing modules (New England Biolabs (NEB)) to generate 3′ dA overhangs at both ends of each fragment. The sample was ligated to the T overhang of the Y-adapter using LNB from Oxford Nanopore Technologies sequencing kit (LSK-SQK109) and T4 DNA Ligase. The samples were purified using Agencourt AMPure XP (Beckman Coulter) beads, with two washes with LFB from Oxford Nanopore Technologies sequencing kit (LSK-SQK109). The ligated substrates were eluted into elution buffer (EB) from the same kit, yielding a ‘DNA library’. DNA libraries were prepared separately using adapters carrying Dda helicases closed with the disulfide linkers as described above.

Electrical measurements were acquired on a custom MinION flow cell and MinION Mk1b from Oxford Nanopore Technologies into which CsgG nanopores were inserted. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

A custom sequencing script was prepared to control the applied potential using the active unblock circuit of the MinION. The sequencing voltage was set to 180 mV and the motor protein was destalled by switching the voltage to zero by disconnecting the channel for 5 sec when the enzyme stall level was detected. Classifications for the stall level and strand (sequencing) level were programmed into a configuration file in the MinKNOW instrument control software that enabled the detection of the stalled species and applied an unblock potential that would not cause full ejection of the strand. The script functioned as follows: if MinKNOW detected that a strand was at the stall level, it would apply the unblock potential for 5 seconds, then return to the sequencing potential of 180 mV to check five times for actively sequencing strands. If the stall level was still present, it would apply the unblock potential for a further 25 seconds, and repeat five times. A rest period of 3 seconds was incorporated between each unblock attempt. If upon returning the sequencing potential, MinKNOW detected an actively sequencing strand, it would stop attempting to unblock and apply only the sequencing potential. If this entire process did not yield an actively sequencing strand, MinKNOW would turn off the channel. The active unblock was set to trigger upon recognising block levels not related to the terminal C3 level, nor the strand, nor open pore, nor enzyme stall levels. Every 15 minutes, a “mux scan” was applied to reset the system, which globally unblocked all channels on the flow cell and checked for active nanopores at 180 mV. Raw data was collected in a bulk FAST5 file using MinKNOW software (Oxford Nanopore Technologies).

Strand-level events from single-channel data that occurred immediately after the C3 level (“C3”, as marked in FIG. 6b) were scored as potential re-reads (e.g., “ii”, as marked in FIG. 6b). These re-reads were confirmed by base calling and comparing the sequence of the re-read to the original read (e.g., “i”, as marked in FIG. 6b), which occurs after the open pore and destalling events, as described in Example 1. Events that were in the same read orientation and within the span of the original read were classed as re-reads. The re-reading efficiency was quantified in two ways: (i) the proportion of reads which dropback and re-read within 30 seconds of reaching the C3 leader, and (ii) the dropback distance, which is the length of the re-read, i.e. the distance the enzyme was pushed back from the C3 leader.

The table below shows the results from this experiment. The results demonstrate re-reading with all linkers tested, and show that an increase in the linker length resulted in an increase in the proportion of reads that have an accompanying re-read within 30 seconds of reaching the C3 leader.

| Disulfide | Number of re- | Proportion of reads | |
|---|---|---|---|
| linker | read events | which dropback | Median dropback |
| chemistry | in run | within 30 seconds | distance (bases) |
| TMAD | 2987 | 0.024 | 747 |
| BMOE | 6334 | 0.049 | 498 |
| BMOP | 547 | 0.155 | 477 |
| BMB | 42 | 0.069 | 529 |
| BM(PEG)₂ | 596 | 0.165 | 427 |

### Example 3

This example demonstrates how a native DNA analyte may be re-read multiple using adapters with different sequences of the leader encountered by a Dda helicase at the 3′ terminus of the sequenced strand.

Y-adapters bearing a leader arm bearing RNA or C3 leader chemistry were prepared by annealing four DNA oligonucleotides with sequences SEQ ID NOs: 50/66, SEQ ID NO: 51, and SEQ ID NO: 52, and a leader oligonucleotide selected from SEQ ID NO: 53, SEQ ID NO: 54 and SEQ ID NO: 55. A DNA motor (a Dda helicase) was loaded onto each adapter, and the disulfide closed via reaction with 1,2-bismaleimidoethane (BMOE). DNA libraries were prepared by ligating the above Y-adapter to E. coli DNA prepared as described in Example 2.

Electrical measurements were acquired on a custom MinION flow cell and MinION Mk1b from Oxford Nanopore Technologies into which CsgG nanopores were inserted. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

Data were collected using a custom script similar to that described in Example 2, except that where the leader contained RNA, re-reads occurred from strand level.

The table below shows the results from this experiment. The results demonstrate re-reading with all leader oligonucleotides tested, and show that an optimum re-reading efficiency was obtained when leader oligonucleotide SEQ ID NO: 55 was used, as judged by a reduction in median time between re-reads.

| Leader | Number of re- | Median time | |
|---|---|---|---|
| oligonucleotide | read events | between re-reads | Median dropback |
| in adapter | in run | (s) | distance (bases) |
| SEQ ID NO: 53 | 209 | 104.0 | 817 |
| SEQ ID NO: 54 | 1719 | 122.6 | 812 |
| SEQ ID NO: 55 | 4016 | 79.4 | 724 |

### Example 4

This example demonstrates how a native DNA analyte may be re-read multiple times at a number of different sequencing run temperatures.

A Y-adapter bearing a leader arm bearing C3 leader chemistry was prepared by annealing four DNA oligonucleotides with sequences SEQ ID NOs: 50/66, SEQ ID NO: 51, SEQ ID NO: 52 and SEQ ID NO: 53. A DNA motor (a Dda helicase) was loaded onto the adapter, and the disulfide closed via reaction with 1,2-bismaleimidoethane (BMOE).

DNA libraries were prepared by ligating the above Y-adapter to E. coli DNA prepared as described in Example 2.

Electrical measurements were acquired on a custom MinION flow cell and MinION Mk1b from Oxford Nanopore Technologies into which CsgG nanopores were inserted. To 1170 μL FB (from Oxford Nanopore Technologies sequencing kit (SQK-LSK109)), 30 μL of FLT was added, yielding tether mix. 800 μL of tether mix was flowed through the system, followed by a 5 minute wait, then a further 200 μL of tether mix flowed through the system with the SpotON port open. 37.5 μL SQB from Oxford Nanopore Technologies sequencing kit (SQK-LSK109), 15 μL of the DNA library and 22.5 μL of LB from Oxford Nanopore Technologies sequencing kit (SQK-LSK109) were mixed, yielding “sequencing mix”. 75 μL of the sequencing mix were added to a MinION flowcell via the SpotON flow cell port.

Data were collected using a custom script similar to that described in Example 2.

The table below shows the results from this experiment. The results demonstrate re-reading at all temperatures tested, and show that the re-reading efficiency increased as the temperature increased, as judged by increases in the proportion of reads that re-read within 30 seconds of reaching the C3 leader, and the median dropback distance.

| | % of strands that | |
|---|---|---|
| | re-read within 30 | |
| | sec of reaching | Median dropback |
| Run temperature | the C3 leader | distance (bases) |
| 34° C. | 5.3 | 467 |
| 37° C. | 9.1 | 584.5 |
| 40° C. | 11.6 | 787 |

### Example 5

This example demonstrates how a DNA analyte may be re-read multiple times by a nanopore, with the movement of DNA through the nanopore controlled by a motor protein.

A Y-adapter was prepared by annealing DNA oligonucleotides 56, 57 and 58. A control Y-adapter was prepared by annealing DNA oligonucleotides 59, 58 and 60. A DNA motor protein (Dda helicase) was loaded onto the adapters and closed by incubation with diamide (TMAD).

Components FB, FLT, SFB, LNB, LB, EB and SQB were obtained from Ligation Sequencing Kit SQK-LSK109 from Oxford Nanopore Technologies plc.

A 3.6-kilobase DNA analyte was obtained via PCR amplification from bacteriophage lambda, then end-repaired and dA-tailed using an Ultra II end-repair and dA-tailing kit (New England Biolabs), to generate 3′ dA overhangs at both ends of each fragment. The sample was ligated to the T overhang of either the Y-adapter or the control Y-adapter using LNB and T4 DNA Ligase, to yield two substrates which were then purified using Agencourt AMPure XP (Beckman Coulter) beads. Beads were washed twice using SFB from Oxford Nanopore Technologies' sequencing kit (SQK-LSK109). The ligated substrates were eluted into elution buffer (EB) from the same kit. Monovalent traptavidin was added to a final concentration of 500 nM, yielding two ‘DNA libraries’.

Electrical measurements were acquired using custom MinION flow cells containing CsgG nanopores, and data collected using a GridION X5 (Oxford Nanopore Technologies plc). To 1170 μL FB, 30 μL of FLT was added, yielding tether mix. 800 μL of tether mix was flowed through the system, followed by a 5 minute wait, then a further 200 μL of tether mix flowed through the system with the SpotON port open. 37.5 μL SQB, 15 μL of each DNA library and 22.5 μL of LB were mixed, yielding “sequencing mix”. 75 μL of the sequencing mix were added to a MinION flowcell via the SpotON flow cell port.

Data were collected using a custom script based on the SQK-LSK109 baseline sequencing script provided for R9.4.1 flow cells by Oxford Nanopore Technologies plc. The script was set up to recognise and accept the open pore, C3 spacer and strand levels, and the active unblock was set to trigger upon recognising the terminal biotin-traptavidin blockade level and other blockades.

An adapter schematic is shown in FIG. 7, A and re-reading schematic shown in FIG. 7, B. Example electrical data are shown in FIG. 8. These data show the capture of a DNA library molecule via the 3′ end, which resulted in a current slightly higher than the open pore current (“leader level”). Following a brief pause at this level, motor-controlled movement was observed at “strand” level, followed by a return to the “leader level”. This pattern (“leader level”, “strand level”) repeated several times. Base calling of the strand-level events confirmed that the same DNA strand was read multiple times without dissociation from the nanopore, and that the controlled movement of the DNA strand out of the pore occurred as far as the leader section, whereupon the motor would “drop back” to an earlier point in the strand (3′ to 5′ movement) and undergo controlled movement (5′ to 3′) to the 3′ end. Where the biotin moiety was not present to act as stopper (in the control adapter), the motor protein was more frequently pushed off the 5′ end of the polynucleotide strand (“push off”).

### Example 6

This example demonstrates how a DNA analyte may be re-read multiple times by a nanopore, with the movement of DNA through the nanopore controlled by a motor protein.

A Y-adapter was prepared by annealing DNA oligonucleotides SEQ ID NOs 61, 62 and 63. A DNA motor protein (Dda helicase) was loaded onto the adapters and closed by incubation with diamide (TMAD).

Components FB, FLT, SFB, LNB, LB, EB and SQB were obtained from Ligation Sequencing Kit SQK-LSK109 from Oxford Nanopore Technologies plc.

NA12878 DNA was fragmented to 180 bp using a Bioruptor Pico (diagenode) following manufacturer's protocols, then end-repaired and dA-tailed using an Ultra II end-repair and dA-tailing kit (New England Biolabs), to generate 3′ dA overhangs at both ends of each fragment. The sample was ligated to the T overhang of the Y-adapter using LNB and T4 DNA Ligase, then purified using Agencourt AMPure XP (Beckman Coulter) beads. Beads were washed twice using SFB from Oxford Nanopore Technologies' sequencing kit (SQK-LSK109). The ligated product was eluted into elution buffer (EB) from the same kit. Monovalent traptavidin was added to a final concentration of 500 nM, yielding ‘DNA library’.

Electrical measurements were acquired using custom MinION flow cells containing CsgG nanopores, and data collected using a GridION X5 (Oxford Nanopore Technologies plc). To 1170 μL FB, 30 μL of FLT was added, yielding tether mix. 800 μL of tether mix was flowed through the system, followed by a 5 minute wait, then a further 200 μL of tether mix flowed through the system with the SpotON port open. 37.5 μL SQB, 15 μL of each DNA library and 22.5 μL of LB were mixed, yielding “sequencing mix”. 75 μL of the sequencing mix were added to a MinION flowcell via the SpotON flow cell port.

Data were collected using a custom script based on the SQK-LSK109 baseline sequencing script provided for R9.4.1 flow cells by Oxford Nanopore Technologies plc. The script was set up to recognise and accept the open pore, C3 spacer and strand levels, and the active unblock was set to trigger upon recognising the terminal biotin-traptavidin blockade level and other blockades.

An adapter schematic is shown in FIG. 7, A and re-reading schematic shown in FIG. 7, B. Example electrical data are shown in FIG. 9. These data show the capture of a DNA library molecule via the 3′ end, which resulted in a current slightly higher than the open pore current (“leader level”). Following a brief pause at this level, motor-controlled movement was observed at “strand” level, followed by a return to the “leader level”. This pattern (“leader level”, “strand level”) repeated several times. Base calling of the strand-level events confirmed that the same DNA strand was read multiple times without dissociation from the nanopore, and that the controlled movement of the DNA strand out of the pore occurred as far as the leader section, whereupon the motor would “drop back” to an earlier point in the strand (3′ to 5′ movement) and undergo controlled movement (5′ to 3′) to the 3′ end.

### Description of the Sequence Listing

SEQ ID NO: 1 shows the amino acid sequence of (hexa-histidine tagged) exonuclease I (EcoExo I) from E. coli.
SEQ ID NO: 2 shows the amino acid sequence of the exonuclease III enzyme from E. coli.
SEQ ID NO: 3 shows the amino acid sequence of the RecJ enzyme from T. thermophilus (TthRecJ-cd).
SEQ ID NO: 4 shows the amino acid sequence of bacteriophage lambda exonuclease. The sequence is one of three identical subunits that assemble into a trimer. (http://www.neb.com/nebecomm/products/productM0262.asp).
SEQ ID NO: 5 shows the amino acid sequence of Phi29 DNA polymerase from Bacillus subtilis phage Phi29.
SEQ ID NO: 6 shows the amino acid sequence of Trwc Cba (Citromicrobium bathyomarinum) helicase.
SEQ ID NO: 7 shows the amino acid sequence of Hel308 Mbu (Methanococcoides burtonii) helicase.
SEQ ID NO: 8 shows the amino acid sequence of the Dda helicase 1993 from Enterobacteria phage T4.
SEQ ID NOs: 9-22, 64, and 65 show the nucleotide sequences of DNA strands discussed in the examples.
SEQ ID NO: 23 shows the amino acid sequence of a preferred HhH domain.
SEQ ID NO: 24 shows the amino acid sequence of the ssb from the bacteriophage RB69, which is encoded by the gp32 gene.
SEQ ID NO: 25 shows the amino acid sequence of the ssb from the bacteriophage T7, which is encoded by the gp2.5 gene.
SEQ ID NO: 26 shows the amino acid sequence of the UL42 processivity factor from Herpes virus 1.
SEQ ID NO: 27 shows the amino acid sequence of subunit 1 of PCNA.
SEQ ID NO: 28 shows the amino acid sequence of subunit 2 of PCNA.
SEQ ID NO: 29 shows the amino acid sequence of subunit 3 of PCNA.
SEQ ID NO: 30 shows the amino acid sequence (from 1 to 319) of the UL42 processivity factor from the Herpes virus 1.
SEQ ID NO: 31 shows the amino acid sequence of the (HhH)2 domain.
SEQ ID NO: 32 shows the amino acid sequence of the (HhH)2-(HhH)2 domain.
SEQ ID NO: 33 shows the amino acid sequence of the human mitochondrial SSB (HsmtSSB).
SEQ ID NO: 34 shows the amino acid sequence of the p5 protein from Phi29 DNA polymerase.
SEQ ID NO: 35 shows the amino acid sequence of the wild-type SSB from E. coli.
SEQ ID NO: 36 shows the amino acid sequence of the ssb from the bacteriophage T4, which is encoded by the gp32 gene.
SEQ ID NO: 37 shows the amino acid sequence of Topoisomerase V Mka (Methanopyrus Kandleri).
SEQ ID NO: 38 shows the amino acid sequence of domains H-L of Topoisomerase V Mka (Methanopyrus Kandleri).
SEQ ID NO: 39 shows the amino acid sequence of Mutant S (Escherichia coli).
SEQ ID NO: 40 shows the amino acid sequence of Sso7d (Sufolobus solfataricus).
SEQ ID NO: 41 shows the amino acid sequence of Sso10b1 (Sulfolobus solfataricus P2).
SEQ ID NO: 42 shows the amino acid sequence of Sso10b2 (Sulfolobus solfataricus P2).
SEQ ID NO: 43 shows the amino acid sequence of Tryptophan repressor (Escherichia coli).
SEQ ID NO: 44 shows the amino acid sequence of Lambda repressor (Enterobacteria phage lambda).
SEQ ID NO: 45 shows the amino acid sequence of Cren7 (Histone crenarchaea Cren7 Sso).
SEQ ID NO: 46 shows the amino acid sequence of human histone (Homo sapiens).
SEQ ID NO: 47 shows the amino acid sequence of dsbA (Enterobacteria phage T4).
SEQ ID NO: 48 shows the amino acid sequence of Rad51 (Homo sapiens).
SEQ ID NO: 49 shows the amino acid sequence of PCNA sliding clamp (Citromicrobium bathyomarinum JL354).
SEQ ID NOs: 50 to 55 and 66 show the polynucleotide sequences of oligonucleotides described in Examples 2 to 4.
SEQ ID NOs: 56 to 60 and 67 to 69 show the polynucleotide sequences of oligonucleotides described in Example 5; in which/5Phos/=5′ phosphate; 3=C3 spacer; 8=Spacer 18; underlined bases=locked nucleic acids (LNA) and/5BioTEG/=5′ biotin TEG.
SEQ ID NOs: 61 to 63 and 70 to 71 show the polynucleotide sequences of oligonucleotides described in Example 6, in which/5Phos/=5′ phosphate; 3=C3 spacer; 8=Spacer 18; underlined bases=locked nucleic acids (LNA) and/5BioTEG/=5′ biotin TEG.

1. A method of characterising a target polynucleotide having a leader attached thereto,
the method comprising:
(i) contacting a detector having a first opening and a second opening or being comprised in a structure having a first opening and a second opening with the leader, under conditions such that the first opening is contacted with the leader and the target polynucleotide moves in a direction from the first opening to the second opening;
the leader having a motor protein bound thereto at a polynucleotide-binding site of the motor protein;
(ii) taking one or more measurements characteristic of the target polynucleotide as the motor protein controls the movement of the target polynucleotide in a first direction with respect to the detector; wherein the first direction is from the second opening to the first opening;
(iii) unbinding the target polynucleotide from the polynucleotide binding site of the motor protein, such that the target polynucleotide moves in a second direction with respect to the detector;
wherein the second direction is from the first opening to the second opening;
(iv) re-binding the target polynucleotide to the polynucleotide binding site of the motor protein;
and taking one or more measurements characteristic of the target polynucleotide as the motor protein controls the movement of the target polynucleotide in the first direction with respect to the detector;
thereby characterising the target polynucleotide.

2. A method according to claim 1, wherein the target polynucleotide has a first end and a second end and the leader is attached at the first end; and the motor protein is oriented in an orientation to process the target polynucleotide in the direction from the second end towards the first end.

3. A method according to claim 1, comprising repeating steps (iii) and (iv) multiple times.

4. A method according to claim 1, wherein prior to step (i) the target polynucleotide is comprised in or consists of a first strand of a double-stranded polynucleotide comprising said first strand and a second strand.

5. A method according to claim 4, wherein the portion of the first strand between the motor protein and the second end is hybridised to the second strand.

6. A method according to claim 4, wherein movement of the target polynucleotide in the direction from the first opening to the second opening comprises separation of the first strand from the second strand.

7. A method according to claim 4, wherein movement of the target polynucleotide in the direction from the second opening to the first opening comprises annealing of the first strand to the second strand.

8. A method according to claim 4, wherein the first strand of the double-stranded polynucleotide is attached to the second strand of the double-stranded polynucleotide.

9. A method according to claim 1, wherein in step (ii) the motor protein controls the movement of a first portion of the target polynucleotide in the first direction with respect to the detector; and in step (iv) the motor protein controls the movement of a second portion of the target polynucleotide in the first direction with respect to the detector; and wherein the first portion at least partially overlaps with the second portion.

10. (canceled)

11. A method according to claim 1, wherein in step (ii) the motor protein controls the movement of a first portion of the target polynucleotide in the first direction with respect to the detector; and in step (iv) the motor protein controls the movement of a second portion of the target polynucleotide in the first direction with respect to the detector; and wherein the first portion does not overlap with the second portion.

12. A method according to claim 1, wherein the distance the target polynucleotide moves with respect to the detector in step (iii) is greater than the distance that the polynucleotide moves with respect to the detector in step (ii) and/or step (iv).

13. A method according to claim 1, wherein (a) in step (iii) the distance the target polynucleotide moves with respect to the detector is at least 1000 nucleotides in length and/or (b) in steps (ii) and/or (iv) the distance the target polynucleotide moves with respect to the detector are each independently at least 100 nucleotides in length.

14. A method according to claim 1, wherein the second end of the target polynucleotide comprises a blocking moiety to prevent the motor protein from disengaging from the polynucleotide; optionally wherein the blocking moiety limits the movement of the target polynucleotide through the polynucleotide binding site of the motor protein and thereby limits the movement of the target polynucleotide in the second direction with respect to the detector.

15. (canceled)

16. A method according to claim 1, wherein the detector comprises a transmembrane nanopore spanning a membrane having a cis side and a trans side, and:
(i) the first opening of the nanopore is at the cis side of the membrane and the second opening of the nanopore is at the trans side; the motor protein controls the movement of the target polynucleotide through the nanopore from the trans side to the cis side of the membrane; and when the target polynucleotide is unbound from the polynucleotide binding site of the motor protein, the target polynucleotide moves through the nanopore from the cis side to the trans side of the membrane; or
(ii) the first opening of the nanopore is at the trans side of the membrane and the second opening of the nanopore is at the cis side; the motor protein controls the movement of the target polynucleotide through the nanopore from the cis side to the trans side of the membrane; and when the target polynucleotide is unbound from the polynucleotide binding site of the motor protein, the target polynucleotide moves through the nanopore from the trans side to the cis side of the membrane

17. A method according to claim 1, wherein the target polynucleotide does not disengage from the motor protein.

18-26. (canceled)

27. A method according to claim 1, wherein prior to step (i) the motor protein is stalled on the leader.

28-30. (canceled)

31. A method according to claim 1, comprising applying a force across the detector, and wherein the motor protein controls the movement of the target polynucleotide with respect to the detector in the direction opposite to the applied force;
optionally wherein said force comprises a voltage potential applied across the detector.

32. A polynucleotide adapter having a first end comprising a leader and a second end comprising an attachment point for attaching to a polynucleotide analyte at a first end of the polynucleotide analyte; wherein said polynucleotide adapter comprises a motor protein stalled thereon in an orientation for processing the adapter in a direction from the second end to the first end.

33. A kit, comprising a first adapter according to claim 32 and a second adapter comprising (i) an attachment point for attaching to a polynucleotide analyte at a second end of the polynucleotide analyte; and (ii) a blocking moiety suitable for preventing the motor protein of the first adapter from disengaging from the polynucleotide analyte when the first adapter is attached to the polynucleotide analyte.

34. A system for characterising a target polynucleotide comprising:
one or more polynucleotide adapters as defined in claim 32;
a nanopore for characterising the target polynucleotide as the target polynucleotide moves with respect to the nanopore; and
a motor protein for controlling the movement of the target polynucleotide with respect to the nanopore.

35. (canceled)
